(19) United States
(12) Patent Application Publication
(10) Pub. No.: US 2025/0084482 A1
(43) Pub. Date: Mar. 13, 2025

(54) METHODS OF USING SOMATIC HLA-I LOH TO PREDICT RESPONSE OF IMMUNE CHECKPOINT INHIBITOR-TREATED PATIENTS WITH LUNG CANCER

(71) Applicant: Foundation Medicine, Inc., Cambridge, MA (US)

(72) Inventors: Meagan Kathleen MONTESION, Somerville, MA (US); Lee Alan ALBACKER, Arlington, MA (US); Karthikeyan MURUGESAN, Boston, MA (US)

(73) Assignee: Foundation Medicine, Inc., Cambridge, MA (US)

(21) Appl. No.: 18/570,006

(22) PCT Filed: Jun. 24, 2022
(86) PCT No.: PCT/US2022/073166

§ 371 Date: Dec. 13, 2023

Related U.S. Application Data
(60) Provisional application No. 63/215,356, filed on Jun. 25, 2021.

(51) Int. Cl. C12Q 1/6886

(52) U.S. Cl. CPC C12Q 1/6886; C12Q 2600/106; C12Q 2600/156

(57) ABSTRACT

Provided herein are methods related to detecting loss of heterozygosity (LOH) of one or more human leukocyte antigen (HLA) genes and/or tumor mutational burden (TMB), as well as methods of treatment and uses related thereto. Detection of LOH of one or more HLA genes and/or TMB can be used to identify individuals that may benefit from treatment with an immune checkpoint inhibitor.

### CROSS REFERENCE TO RELATED APPLICATIONS

This application claims the priority benefit of U.S. Provisional Application No. 63/215,356, filed Jun. 25, 2021, which is incorporated herein by reference in its entirety.

### TECHNICAL FIELD

Provided herein are methods related to detecting loss of heterozygosity (LOH) of one or more human leukocyte antigen (HLA) genes and/or tumor mutational burden (TMB) in cancer, as well as methods of diagnosis/treatment and uses related thereto.

### BACKGROUND

Immunotherapies have revolutionized current treatments for advanced cancer patients. Some (e.g., cell-based therapies) provide or stimulate an immune response to the cancer, while others (e.g., immune checkpoint inhibitors or ICIs) are thought to reinvigorate the patient's own T-cell mediated immune response (Reck, M., et al. N Engl J Med 375, 1823-1833 (2016); Hellmann, M. D., et al. N Engl J Med 378, 2093-2104 (2018); Nghiem, P. T., et al. N Engl J Med 374, 2542-2552 (2016); Robert, C., et al. N Engl J Med 372, 2521-2532 (2015); Le, D. T., et al. N Engl J Med 372, 2509-2520 (2015)). However, responses to immunotherapies such as ICI treatment have been found to be variable among different patients.

The adaptive immune system can recognize cancer cells via the presentation of tumor-specific mutant peptides (neoantigens) presented on human leukocyte antigen class I (HLA-I) gene-encoded major histocompatibility complex class I (MHC-I) proteins (Mok, T. S. K., et al. Lancet 393, 1819-1830 (2019); Schumacher, T. N. & Schreiber, R. D. Science 348, 69-74 (2015); Turajlic, S., et al. Lancet Oncol 18, 1009-1021 (2017)). It has been suggested that loss of heterozygosity (LOH) of one or more HLA-I genes could facilitate immune evasion by tumors, potentially resulting in a decreased ability to present neoantigens to the adaptive immune system (see, e.g., McGranahan et al., Cell (2017) 171(6): 1259-1271.e11).

Thus, there is a need in the art for characterizing LOH of one or more HLA-I genes in cancer, and for determining the effects of LOH of one or more HLA-I genes (alone or in combination with other biomarkers, such as tumor mutational burden) on responses to anti-cancer treatments such as ICIs.

All references cited herein, including patent applications and publications, are incorporated by reference in their entirety.

### SUMMARY OF THE INVENTION

In one aspect, provided herein is a method of identifying an individual having a squamous cell cancer or a non-small cell lung cancer (NSCLC) who may benefit from a treatment comprising an immune checkpoint inhibitor, the method comprising detecting in a sample from the individual: (a) a somatic loss of heterozygosity (LOH) of one or more human leukocyte antigen class I (HLA-I) genes, or (b) a somatic LOH of one or more HLA-I genes and a high tumor mutational burden (TMB), wherein detection in the sample of the somatic LOH of one or more HLA-I genes, or of the somatic LOH of one or more HLA-I genes and high TMB, identifies the individual as one who may benefit from a treatment comprising an immune checkpoint inhibitor.

In another aspect, provided herein is a method of detecting the presence or absence of a squamous cell cancer or a NSCLC in an individual, the method comprising: (a) detecting the presence or absence of a squamous cell cancer or a NSCLC in a sample from the individual; and (b) detecting in a sample from the individual the presence or absence of: (i) a somatic LOH of one or more HLA-I genes, or (ii) a somatic LOH of one or more HLA-I genes and a high TMB.

In another aspect, provided herein is a method of selecting a therapy for an individual having a squamous cell cancer or a NSCLC, the method comprising detecting in a sample from the individual: (a) a somatic LOH of one or more HLA-I genes, or (b) a somatic LOH of one or more HLA-I genes and a high TMB, wherein detection in the sample of the somatic LOH of one or more HLA-I genes, or of the somatic LOH of one or more HLA-I genes and high TMB, identifies the individual as one who may benefit from a treatment comprising an immune checkpoint inhibitor.

In another aspect, provided herein is a method of identifying one or more treatment options for an individual having a squamous cell cancer or a NSCLC, the method comprising: (a) detecting in a sample from the individual: (i) a somatic LOH of one or more HLA-I genes, or (ii) a somatic LOH of one or more HLA-I genes and a high TMB; and (b) generating a report comprising one or more treatment options identified for the individual based at least in part on the detection in the sample of the somatic LOH of one or more HLA-I genes, or of the somatic LOH of one or more HLA-I genes and high TMB, wherein the one or more treatment options comprise an immune checkpoint inhibitor.

In another aspect, provided herein is a method of identifying one or more treatment options for an individual having a squamous cell cancer or a NSCLC, the method comprising: (a) acquiring knowledge of: (i) a somatic LOH of one or more HLA-I genes, or (ii) a somatic LOH of one or more HLA-I genes and a high TMB, in a sample from the individual; and (b) generating a report comprising one or more treatment options identified for the individual based at least in part on said knowledge, wherein the one or more treatment options comprise an immune checkpoint inhibitor.

In another aspect, provided herein is a method of selecting a treatment for an individual having a squamous cell cancer or a NSCLC, comprising: acquiring knowledge of: (i) a somatic LOH of one or more HLA-I genes, or (ii) a somatic LOH of one or more HLA-I genes and a high TMB, in a sample from the individual; wherein responsive to the acquisition of said knowledge: (i) the individual is classified as a candidate to receive a treatment comprising an immune checkpoint inhibitor; and/or (ii) the individual is identified as likely to respond to a treatment that comprises an immune checkpoint inhibitor.

In another aspect, provided herein is a method of predicting survival of an individual having a squamous cell cancer or a NSCLC, comprising: acquiring knowledge of: (i) a somatic LOH of one or more HLA-I genes, or (ii) a somatic LOH of one or more HLA-I genes and a high TMB, in a sample from the individual, wherein responsive to the acquisition of said knowledge, the individual is predicted to have longer survival when treated with a treatment comprising an immune checkpoint inhibitor, as compared to survival of an individual whose squamous cell cancer or NSCLC does not comprise the somatic LOH of one or more HLA-I genes, or the somatic LOH of one or more HLA-I genes and high TMB.

In another aspect, provided herein is a method of predicting survival of an individual having a squamous cell cancer or a NSCLC treated with an immune checkpoint inhibitor, the method comprising: acquiring knowledge of: (i) a somatic LOH of one or more HLA-I genes, or (ii) a somatic LOH of one or more HLA-I genes and a high TMB, in a sample from the individual, wherein responsive to the acquisition of said knowledge, the individual is predicted to have longer survival after a treatment comprising an immune checkpoint inhibitor, as compared to an individual whose cancer does not exhibit the somatic LOH of one or more HLA-I genes, or the somatic LOH of one or more HLA-I genes and high TMB.

In another aspect, provided herein is a method of treating or delaying progression of a squamous cell cancer or a NSCLC, comprising: (a) acquiring knowledge of: (i) a somatic LOH of one or more HLA-I genes, or (ii) a somatic LOH of one or more HLA-I genes and a high TMB, in a sample from an individual having a squamous cell cancer or a NSCLC; and (b) responsive to said knowledge, administering to the individual an effective amount of a treatment that comprises an immune checkpoint inhibitor.

In another aspect, provided herein is a method of treating or delaying progression of a squamous cell cancer or a NSCLC, comprising, responsive to acquiring knowledge of: (i) a somatic LOH of one or more HLA-I genes, or (ii) a somatic LOH of one or more HLA-I genes and a high TMB, in a sample from an individual having a squamous cell cancer or a NSCLC, administering to the individual an effective amount of a treatment that comprises an immune checkpoint inhibitor.

In another aspect, provided herein is a method of monitoring, evaluating, or screening an individual having a squamous cell cancer or a NSCLC, comprising: acquiring knowledge of: (i) a somatic LOH of one or more HLA-I genes, or (ii) a somatic LOH of one or more HLA-I genes and a high TMB, in a sample from the individual, wherein responsive to the acquisition of said knowledge, the individual is predicted to have longer survival when treated with a treatment comprising an immune checkpoint inhibitor, as compared to an individual whose squamous cell cancer or NSCLC does not comprise the somatic LOH of one or more HLA-I genes, or the somatic LOH of one or more HLA-I genes and high TMB.

In another aspect, provided herein is a method of treating or delaying progression of a squamous cell cancer or a NSCLC, comprising: (a) detecting in a sample from an individual having a squamous cell cancer or a NSCLC: (i) a somatic LOH of one or more HLA-I genes, or (ii) a somatic LOH of one or more HLA-I genes and a high TMB; and (b) administering to the individual an effective amount of a treatment that comprises an immune checkpoint inhibitor.

In another aspect, provided herein is a method of assessing a squamous cell cancer or a NSCLC in an individual, the method comprising: (a) detecting in a sample from the individual: (i) a somatic LOH of one or more HLA-I genes, or (ii) a somatic LOH of one or more HLA-I genes and a high TMB; and (b) providing an assessment of the somatic LOH of one or more HLA-I genes, or the somatic LOH of one or more HLA-I genes and high TMB, in the squamous cell cancer or NSCLC.

In some embodiments of any of the methods provided herein, acquiring knowledge of the somatic LOH of one or more HLA-I genes, or of the somatic LOH of one or more HLA-I genes and high TMB, comprises detecting the somatic LOH of one or more HLA-I genes, or the somatic LOH of one or more HLA-I genes and high TMB, in a sample from the individual.

In some embodiments of any of the methods provided herein, detecting somatic LOH of one or more HLA-I genes comprises: providing a plurality of nucleic acids obtained from a sample from the individual, wherein the plurality of nucleic acids comprises nucleic acids encoding an HLA-I gene; optionally, ligating one or more adaptors onto one or more nucleic acids from the plurality of nucleic acids; amplifying nucleic acids from the plurality of nucleic acids; capturing a plurality of nucleic acids corresponding to the HLA-I gene, wherein the plurality of nucleic acids corresponding to the HLA-I gene is captured from the amplified nucleic acids by hybridization with a bait molecule; sequencing, by a sequencer, the captured nucleic acids to obtain a plurality of sequence reads corresponding to the HLA-I gene; fitting, by one or more processors, one or more values associated with one or more of the plurality of sequence reads to a model; and based on the model, detecting the somatic LOH of one or more HLA-I genes and a relative binding propensity for an HLA allele of the HLA-I gene. In some embodiments, the somatic LOH of one or more HLA-I genes and relative binding propensity for an HLA allele of the HLA-I gene are detected by: (a) obtaining an observed allele frequency for an HLA allele of an HLA-I gene, wherein the observed allele frequency corresponds to the frequency of nucleic acid(s) encoding at least a portion of the HLA allele as detected among the plurality of sequence reads corresponding to the HLA-I gene; (b) obtaining a relative binding propensity for the HLA allele to the bait molecule, wherein the relative binding propensity of the HLA allele corresponds to propensity of a nucleic acid encoding at least a portion of the HLA allele to bind the bait molecule in the presence of nucleic acids encoding portions of one or more other HLA alleles; (c) applying an objective function to measure a difference between the relative binding propensity and the observed allele frequency of the HLA allele; (d) applying an optimization model to minimize the objective function; (e) determining an adjusted allele frequency of the HLA allele based on the optimization model and the observed allele frequency; and (f) determining that LOH of one or more HLA-I genes has occurred when the adjusted allele frequency of the HLA allele is less than a predetermined threshold. In some embodiments, the plurality of sequence reads is obtained by whole exome sequencing, whole genome sequencing, gene-targeted sequencing, or next-generation sequencing. In other embodiments, the plurality of sequence reads is obtained by next-generation sequencing.

In some embodiments of any of the methods provided herein, detecting somatic LOH of one or more HLA-I genes comprises determining the specific copy number of an HLA allele of the one or more HLA-I genes in the squamous cell cancer or NSCLC. In some embodiments, detecting somatic LOH of one or more HLA-I genes further comprises: (a) aligning a plurality of sequence reads of an HLA allele of one or more HLA-I genes with reference sequence reads of an HLA allele of one or more HLA-I genes, wherein the plurality of sequence reads is derived from a sample of the squamous cell cancer or NSCLC, and wherein the reference sequence reads are based on the individual's HLA type; (b) determining mismatch positions in homologous HLA alleles of the one or more HLA-I genes, and determining mismatch coverage for each HLA allele; (c) determining the ratio and allele frequency of each HLA allele based on mismatches and coverage determined in step (b); and (d) determining the copy number of each HLA allele in the squamous cell cancer or NSCLC based on the ratio and allele frequency determined in step (c). In some embodiments, the plurality of sequence reads is obtained by whole exome sequencing, whole genome sequencing, gene-targeted sequencing, or next-generation sequencing. In other embodiments, the plurality of sequence reads is obtained by next-generation sequencing.

In some embodiments of any of the methods provided herein, somatic LOH of one or more HLA-I genes is detected by sequencing. In some embodiments of any of the methods provided herein, somatic LOH of one or more HLA-I genes is detected by whole exome sequencing, whole genome sequencing, gene-targeted sequencing, or next-generation sequencing. In some embodiments of any of the methods provided herein, somatic LOH of one or more HLA-I genes is detected by next-generation sequencing.

In some embodiments of any of the methods provided herein, the one or more HLA-I genes comprise one or more of a human HLA-A, HLA-B or HLA-C gene.

In some embodiments of any of the methods provided herein, a high TMB comprises a TMB of at least about 10 mutations/megabase (mut/Mb). In some embodiments of any of the methods provided herein, a high TMB is detected by sequencing, whole exome sequencing, whole genome sequencing, gene-targeted sequencing, or next-generation sequencing.

In some embodiments of any of the methods provided herein, the squamous cell cancer or NSCLC is PD-L1-positive. In some embodiments, the squamous cell cancer or NSCLC has a tumor proportion score of at least about 1%. In other embodiments, at least about 1% of tumor cells in a sample obtained from the squamous cell cancer or NSCLC are PD-L1-positive. In some embodiments, PD-L1 positivity is assessed by immunohistochemistry. In some embodiments, PD-L1 positivity is assessed in a sample comprising squamous cell cancer or NSCLC cells obtained from the individual.

In some embodiments of any of the methods provided herein, the squamous cell cancer or NSCLC has a tumor mutational burden of at least about 10 mut/Mb.

In some embodiments of any of the methods provided herein, the squamous cell cancer or NSCLC does not comprise a mutation in an EGFR gene and/or an ALK gene. In some embodiments of any of the methods provided herein, the squamous cell cancer or NSCLC is EGFR-wild type and/or ALK-wild type. In some embodiments of any of the methods provided herein, the squamous cell cancer or NSCLC does not comprise a pathogenic mutation in an EGFR gene and/or an ALK gene.

In some embodiments of any of the methods provided herein, the squamous cell cancer or NSCLC is an advanced squamous cell cancer or NSCLC. In some embodiments of any of the methods provided herein, the squamous cell cancer or NSCLC is a metastatic squamous cell cancer or NSCLC.

In some embodiments of any of the methods provided herein, the NSCLC is an adenocarcinoma, a squamous cell cancer, a large cell cancer, an undifferentiated cancer, a carcinoid tumor, a pleomorphic salivary gland cancer, an adenosquamous cancer, sarcomatoid cancer, or an unclassified carcinoma. In some embodiments, the NSCLC is an adenocarcinoma or a squamous cell cancer.

In some embodiments of any of the methods provided herein, the squamous cell cancer is a skin, lip, mouth, esophageal, head and neck, urinary tract, thyroid, penis, prostate, bladder, lung, vaginal, or cervical cancer. In some embodiments, the squamous cell cancer is a non-melanoma skin cancer. In some embodiments, the squamous cell cancer is a head and neck cancer. In some embodiments, the squamous cell cancer is an esophageal cancer. In some embodiments, the squamous cell cancer is a squamous cell lung cancer. In some embodiments, the squamous cell lung cancer comprises a mutation in a CDKN2A gene, a SOX2 gene, an LRP1B gene, a BRCA1 gene, an FGF12 gene, a TERC gene, a PIK3CA gene, a PRKCI gene, a PTEN gene an ARID1A gene, a KDM5A gene, a SPTA1 gene, a FAS gene, an FUBP1 gene, or any combination thereof. In some embodiments, the squamous cell lung cancer comprises a tobacco signature. In some embodiments, the squamous cell lung cancer is a non-small cell lung cancer (NSCLC).

In some embodiments of any of the methods provided herein, the squamous cell cancer or NSCLC was previously treated with an immune checkpoint inhibitor. In some embodiments of any of the methods provided herein, the squamous cell cancer or NSCLC was previously treated with an anti-cancer therapy other than an immune checkpoint inhibitor. In some embodiments of any of the methods provided herein, the squamous cell cancer or NSCLC was not previously treated with an immune checkpoint inhibitor. In some embodiments of any of the methods provided herein, the squamous cell cancer or NSCLC was not previously treated with an anti-cancer therapy other than an immune checkpoint inhibitor. In some embodiments of any of the methods provided herein, the squamous cell cancer or NSCLC was not previously treated. In some embodiments of any of the methods provided herein, the squamous cell cancer or NSCLC was previously treated with a first line anti-cancer therapy for squamous cell cancer or NSCLC. In some embodiments, the first line anti-cancer therapy comprises carboplatin, paclitaxel, paclitaxel protein-bound, gemcitabine, docetaxel, ramucirumab, or any combination thereof. In some embodiments of any of the methods provided herein, the squamous cell cancer or NSCLC was previously treated with a second line anti-cancer therapy for squamous cell cancer or NSCLC. In some embodiments of any of the methods provided herein, the squamous cell cancer or NSCLC was previously treated with a first line immune checkpoint inhibitor for squamous cell cancer or NSCLC. In some embodiments of any of the methods provided herein, the squamous cell cancer or NSCLC was previously treated with a second line immune checkpoint inhibitor for squamous cell cancer or NSCLC. In some embodiments of any of the methods provided herein, the immune checkpoint inhibitor is a monotherapy. In some embodiments of any of the methods provided herein, the immune checkpoint inhibitor is a first line immune checkpoint inhibitor. In some embodiments of any of the methods provided herein, the immune checkpoint inhibitor is a second line immune checkpoint inhibitor.

In some embodiments of any of the methods provided herein, the immune checkpoint inhibitor is a PD-1- or a PD-L1-targeted agent. In some embodiments, the immune checkpoint inhibitor is a PD-1 inhibitor. In some embodiments, the immune checkpoint inhibitor comprises one or more of nivolumab, pembrolizumab, cemiplimab, or dostarlimab. In other embodiments, the immune checkpoint inhibitor is a PD-L1-inhibitor. In some embodiments, the immune checkpoint inhibitor comprises one or more of atezolizumab, avelumab, or durvalumab.

In some embodiments of any of the methods provided herein, the immune checkpoint inhibitor is a CTLA-4 inhibitor. In some embodiments, the CTLA-4 inhibitor comprises ipilimumab.

In some embodiments of any of the methods provided herein, the treatment or the one or more treatment options further comprise an additional anti-cancer therapy. In some embodiments, the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof. In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy. In some embodiments, the nucleic acid comprises a double-stranded RNA (dsRNA), a small interfering RNA (siRNA), or a small hairpin RNA (shRNA).

In some embodiments of any of the methods provided herein, the sample is obtained from the squamous cell cancer or NSCLC.

In some embodiments of any of the methods provided herein, the sample comprises cells from the squamous cell cancer or NSCLC and/or nucleic acids from the squamous cell cancer or NSCLC. In some embodiments, the sample further comprises non-squamous cell cancer or non-NSCLC cells and/or non-squamous cell cancer or non-NSCLC nucleic acids.

In some embodiments of any of the methods provided herein, the sample is from a tumor biopsy, tumor specimen, or circulating tumor cell. In some embodiments, the sample comprises cell-free DNA (cfDNA) or circulating tumor DNA (ctDNA) from the squamous cell cancer or NSCLC. In some embodiments, the sample comprises fluid, cells, or tissue. In some embodiments, the sample comprises blood or plasma.

In some embodiments of any of the methods provided herein, the sample is a nucleic acid sample. In some embodiments, the nucleic acid sample comprises mRNA, DNA, circulating tumor DNA, cell-free DNA, or cell-free RNA.

In some embodiments of any of the methods provided herein, the individual is a human.

In another aspect, provided herein is an immune checkpoint inhibitor for use in a method of treating or delaying progression of a squamous cell cancer or NSCLC, wherein the method comprises administering the immune checkpoint inhibitor to an individual, wherein a somatic LOH of one or more HLA-I genes, or a somatic LOH of one or more HLA-I genes and high TMB are detected in a sample derived from a squamous cell cancer or NSCLC in the individual.

In another aspect, provided herein is an immune checkpoint inhibitor for use in the manufacture of a medicament for treating or delaying progression of a squamous cell cancer or NSCLC, wherein the medicament is to be administered to an individual, wherein a somatic LOH of one or more HLA-I genes, or a somatic LOH of one or more HLA-I genes and high TMB are detected in a sample derived from a squamous cell cancer or NSCLC in the individual.

In another aspect, provided herein is a system, comprising: a memory configured to store one or more program instructions; and one or more processors configured to execute the one or more program instructions, wherein the one or more program instructions when executed by the one or more processors are configured to: (a) obtain a plurality of sequence reads of one or more nucleic acids, wherein the one or more nucleic acids are derived from a sample of a squamous cell cancer or NSCLC obtained from an individual; (b) analyze the plurality of sequence reads for the presence of a somatic LOH of one or more HLA-I genes, or a somatic LOH of one or more HLA-I genes and a high TMB; (c) detect, based on the analyzing, the presence of a somatic LOH of one or more HLA-I genes, or a somatic LOH of one or more HLA-I genes and high TMB, in the sample; and (d) generate, based at least in part on the detecting, a genomic profile for the sample. In some embodiments, the analyzing comprises: (a) determining an observed allele frequency for an HLA allele of an HLA-I gene, wherein the observed allele frequency corresponds to the frequency of nucleic acid(s) encoding at least a portion of the HLA allele as detected among a plurality of sequence reads corresponding to an HLA-I gene, wherein the plurality of sequence reads was obtained by sequencing nucleic acids encoding the HLA-I gene or a portion thereof as captured by hybridization with a bait molecule; (b) determining a relative binding propensity for the HLA allele to the bait molecule, wherein the relative binding propensity of the HLA allele corresponds to propensity of a nucleic acid encoding at least a portion of the HLA allele to bind the bait molecule in the presence of nucleic acids encoding portions of one or more other HLA alleles; (c) executing an objective function to measure a difference between the relative binding propensity and the observed allele frequency of the HLA allele; (d) executing an optimization model to minimize the objective function; (e) determining an adjusted allele frequency of the HLA allele based on the optimization model and the observed allele frequency; and (f) determining the presence of a somatic LOH of one or more HLA-I genes when the adjusted allele frequency of the HLA allele is less than a predetermined threshold.

In another aspect, provided herein is a non-transitory computer readable storage medium comprising one or more programs executable by one or more computer processors for performing a method, comprising: (a) obtaining, using the one or more processors, a plurality of sequence reads of one or more nucleic acids, wherein the one or more nucleic acids are derived from a sample of a squamous cell cancer or NSCLC obtained from an individual; (b) analyzing, using the one or more processors, the plurality of sequence reads for the presence of a somatic LOH of one or more HLA-I genes, or a somatic LOH of one or more HLA-I genes and a high TMB; (c) detecting, using the one or more processors and based on the analyzing, the presence of a somatic LOH of one or more HLA-I genes, or of a somatic LOH of one or more HLA-I genes and high TMB, in the sample; and (d) generating, based at least in part on the detecting, a genomic profile for the sample. In some embodiments, the analyzing comprises: receiving, using the one or more processors, an observed allele frequency for an HLA allele of an HLA-I gene, wherein the observed allele frequency corresponds to the frequency of nucleic acid(s) encoding at least a portion of the HLA allele as detected among a plurality of sequence reads corresponding to an HLA-I gene, wherein the plurality of sequence reads was obtained by sequencing nucleic acids encoding the HLA-I gene or a portion thereof as captured by hybridization with a bait molecule; receiving, using the one or more processors, a relative binding propensity for the HLA allele to the bait molecule, wherein the relative binding propensity of the HLA allele corresponds to propensity of a nucleic acid encoding at least a portion of the HLA allele to bind the bait molecule in the presence of nucleic acids encoding portions of one or more other HLA alleles; executing, using the one or more processors, an objective function to measure a difference between the relative binding propensity and the observed allele frequency of the HLA allele; executing, using the one or more processors, an optimization model to minimize the objective function; determining, using the one or more processors, an adjusted allele frequency of the HLA allele based on the optimization model and the observed allele frequency; and determining, using the one or more processors, that a somatic LOH of one or more HLA-I genes has occurred when the adjusted allele frequency of the HLA allele is less than a predetermined threshold.

In some embodiments of any of the systems or non-transitory computer readable storage media provided herein, the one or more HLA-I genes comprise one or more of a human HLA-A, HLA-B, or HLA-C gene.

In some embodiments of any of the systems or non-transitory computer readable storage media provided herein, the plurality of sequence reads is obtained by sequencing nucleic acids obtained from a sample comprising squamous cell cancer or NSCLC cells and/or squamous cell cancer or NSCLC nucleic acids. In some embodiments, the plurality of sequence reads is obtained by whole exome sequencing, whole genome sequencing, gene-targeted sequencing, or next-generation sequencing. In some embodiments, the sample further comprises non-squamous cell cancer or non-NSCLC cells and/or non-squamous cell cancer or non-NSCLC nucleic acids. In some embodiments, the sample is from a tumor biopsy, tumor specimen, or a circulating tumor cell. In some embodiments, the sample comprises cell-free DNA (cfDNA) or circulating tumor DNA (ctDNA) from the squamous cell cancer or NSCLC. In some embodiments, the sample comprises fluid, cells, or tissue. In some embodiments, the sample comprises blood or plasma. In some embodiments, the sample is a nucleic acid sample. In some embodiments, the nucleic acid sample comprises mRNA, DNA, circulating tumor DNA, cell-free DNA, or cell-free RNA. In some embodiments, a high TMB comprises a TMB of at least about 10 mut/Mb.

In some embodiments of any of the systems or non-transitory computer readable storage media provided herein, the individual is administered a treatment based at least in part on the genomic profile.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE FIGURES

FIG. 1 is a schematic depiction of a hybrid capture process.

FIG. 2 illustrates the result of a bias removal process.

FIG. 3 illustrates methodological considerations for detection of HLA-I LOH due to baiting effects, including the bait/target sequence divergence effects in B-allele frequency (BAF; upper), and the modeled BAF accounting for sequencing (lower).

FIG. 4 shows a dendrogram of representative sequences for each known two-digit haplotype of HLA-A. A matrix of all pairwise sequence distances was used to cluster the haplotypes. The k affinity constants for haplotypes on the left were all greater than or equal to 1, while the k constants for sequences on the right were greater than 0.7 or between 0.7 and 0.9. The dot on the left axis represents the sequence of a specific bait molecule used for capture of various HLA alleles.

FIG. 5 depicts a block diagram of an exemplary process for detecting loss-of-heterozygosity (LOH) of a human leukocyte antigen (HLA) gene, in accordance with some embodiments.

FIG. 6 depicts a block diagram of an exemplary process for identifying relative binding propensities of different alleles of a polymorphic gene to a bait molecule, in accordance with some embodiments.

FIG. 7 depicts a block diagram of an exemplary process for determining allele frequency, in accordance with some embodiments.

FIG. 8 depicts an exemplary device, in accordance with some embodiments.

FIG. 9 depicts an exemplary system, in accordance with some embodiments.

FIG. 10 depicts a block diagram of an exemplary process for detecting LOH of one or more HLA-I genes, or LOH of one or more HLA-I genes and high TMB, in accordance with some embodiments.

FIG. 11 shows the overall survival for squamous cell lung carcinoma (lung SCC) patients with loss of heterozygosity of an HLA-I gene (“HLA-I LOH”; N=33) or without HLA-I LOH (“HLA-I Intact”; N=66). Survival is shown from the start of second line immune checkpoint inhibitor (ICI) monotherapy. The dotted lines in the survival plot (top panel) represent the median overall survival. The bottom panel shows the number of patients at risk stratified by HLA-I LOH status.

FIG. 12 shows the overall survival for lung SCC patients with HLA-I LOH (“LOH”) or without HLA-I LOH (“Intact”) stratified with high tumor mutational burden (TMB; “High”) or without high TMB (“Low”). Cohorts include: patients with HLA-I LOH and with high tumor mutational burden (“High/LOH”; N=25), patients without HLA-I LOH and with high tumor mutational burden (“High/Intact”; N=39), patients with HLA-I LOH and without high tumor mutational burden (“Low/LOH”; N=12), and patients without HLA-I LOH and without high tumor mutational burden (“Low/Intact”; N=33). Survival is shown from the start of second line ICI monotherapy. The dotted lines in the survival plot (top panel) represent the median overall survival. A high TMB was defined as ≥10 mutations/megabase (mut/Mb). The bottom panel shows the number of patients at risk stratified by HLA-I LOH and TMB status.

FIG. 13 shows the hazard ratio for lung SCC patients stratified by HLA-I LOH and TMB status. Survival was assessed from the start of second line ICI monotherapy. TMB High was defined as TMB ≥10 mut/Mb.

FIG. 14 shows the overall survival for lung SCC patients with HLA-I LOH (N=24) or without HLA-I LOH (“HLA-I Intact”; N=46). Survival is shown from the start of first line ICI monotherapy. The dotted lines in the survival plot (top panel) represent the median overall survival. The bottom panel shows the number of patients at risk stratified by HLA-I LOH status.

FIG. 15 shows a volcano plot of biomarkers enriched in lung SCC samples with HLA-I LOH (“HLA-I LOH Positive Lung SCC”) or without HLA-I LOH (“HLA-I LOH Negative Lung SCC”). n=807 for HLA-I LOH Positive Lung SCC and n=1768 for HLA-I LOH Negative Lung SCC.

### DETAILED DESCRIPTION

The present disclosure relates generally to detecting loss of heterozygosity (LOH) of one or more human leukocyte antigen (HLA) genes and/or tumor mutational burden (TMB) in squamous cell cancer or non-small cell lung cancer (NSCLC), as well as methods of treatment, and uses related thereto.

The present disclosure describes a study of the responses of squamous cell cancer or NSCLC patients, e.g., squamous cell lung cancer or squamous NSCLC patients, to treatment with immune checkpoint inhibitors, stratified by somatic loss of heterozygosity (LOH) of one or more HLA class I (HLA-I) genes, e.g., an HLA-A, HLA-B, or HLA-C gene, and/or tumor mutational burden (TMB). As described herein, Applicants found that, unexpectedly, LOH of one or more HLA-I genes is an independent and significant positive predictor of survival in patients treated with immune checkpoint inhibitors. Applicants further found that, unexpectedly, LOH of one or more HLA-I genes and a high TMB are together also associated with longer survival of patients treated with immune checkpoint inhibitors. Accordingly, without wishing to be bound by theory, it is thought that the presence of LOH of one or more HLA-I genes, or of LOH of one or more HLA-I genes and a high TMB, may identify squamous cell cancer or NSCLC patients, e.g., squamous cell lung cancer or squamous NSCLC patients, who are likely to respond to immune checkpoint inhibitors.

### I. GENERAL TECHNIQUES

The techniques and procedures described or referenced herein are generally well understood and commonly employed using conventional methodology by those skilled in the art, such as, for example, the widely utilized methodologies described in Sambrook et al., Molecular Cloning: A Laboratory Manual 3d edition (2001) Cold Spring Harbor Laboratory Press, Cold Spring Harbor, N.Y.; Current Protocols in Molecular Biology (F. M. Ausubel, et al. eds., (2003)); the series Methods in Enzymology (Academic Press, Inc.): PCR 2: A Practical Approach (M. J. MacPherson, B. D. Hames and G. R. Taylor eds. (1995)), Harlow and Lane, eds. (1988) Antibodies, A Laboratory Manual, and Animal Cell Culture (R. I. Freshney, ed. (1987)); Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney), ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Handbook of Experimental Immunology (D. M. Weir and C. C. Blackwell, eds.); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: A Practical Approach (D. Catty., ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane (Cold Spring Harbor Laboratory Press, 1999); The Antibodies (M. Zanetti and J. D. Capra, eds., Harwood Academic Publishers, 1995); and Cancer: Principles and Practice of Oncology (V. T. DeVita et al., eds., J. B. Lippincott Company, 1993).

### II. DEFINITIONS

As used in this specification and the appended claims, the singular forms “a”, “an” and “the” include plural referents unless the context clearly dictates otherwise. Thus, for example, reference to “a molecule” optionally includes a combination of two or more such molecules, and the like.

The term “about” as used herein refers to the usual error range for the respective value readily known to the skilled person in this technical field. Reference to “about” a value or parameter herein includes (and describes) embodiments that are directed to that value or parameter per se.

It is understood that aspects and embodiments of the invention described herein include “comprising,” “consisting,” and “consisting essentially of” aspects and embodiments.

The terms “cancer” and “cancerous” refer to or describe the physiological condition in mammals that is typically characterized by unregulated cell growth. Included in this definition are benign and malignant cancers.

The term “tumor,” as used herein, refers to all neoplastic cell growth and proliferation, whether malignant or benign, and all pre-cancerous and cancerous cells and tissues. The terms “cancer,” “cancerous,” and “tumor” are not mutually exclusive as referred to herein.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase, or by a synthetic reaction. Thus, for instance, polynucleotides as defined herein include, without limitation, single- and double-stranded DNA, DNA including single- and double-stranded regions, single- and double-stranded RNA, and RNA including single- and double-stranded regions, hybrid molecules comprising DNA and RNA that may be single-stranded or, more typically, double-stranded or include single- and double-stranded regions. In addition, the term “polynucleotide” as used herein refers to triple-stranded regions comprising RNA or DNA or both RNA and DNA. The strands in such regions may be from the same molecule or from different molecules. The regions may include all of one or more of the molecules, but more typically involve only a region of some of the molecules. One of the molecules of a triple-helical region often is an oligonucleotide. The term “polynucleotide” specifically includes cDNAs.

A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after synthesis, such as by conjugation with a label. Other types of modifications include, for example, “caps,” substitution of one or more of the naturally-occurring nucleotides with an analog, internucleotide modifications such as, for example, those with uncharged linkages (e.g., methyl phosphonates, phosphotriesters, phosphoamidates, carbamates, and the like) and with charged linkages (e.g., phosphorothioates, phosphorodithioates, and the like), those containing pendant moieties, such as, for example, proteins (e.g., nucleases, toxins, antibodies, signal peptides, poly-L-lysine, and the like). those with intercalators (e.g., acridine, psoralen, and the like), those containing chelators (e.g., metals, radioactive metals, boron, oxidative metals, and the like), those containing alkylators, those with modified linkages (e.g., alpha anomeric nucleic acids), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid or semi-solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-0-methyl-, 2′-0-allyl-, 2′-fluoro-, or 2′-azido-ribose, carbocyclic sugar analogs, a-anomeric sugars, epimeric sugars such as arabinose, xyloses or lyxoses, pyranose sugars, furanose sugars, sedoheptuloses, acyclic analogs, and abasic nucleoside analogs such as methyl riboside. One or more phosphodiester linkages may be replaced by alternative linking groups. These alternative linking groups include, but are not limited to, embodiments wherein phosphate is replaced by P(0)S (“thioate”), P(S)S (“dithioate”), “(0)NR₂ (“amidate”), P(O)R, P(0)OR′, CO or CH₂ (“formacetal”), in which each R or R′ is independently H or substituted or unsubstituted alkyl (1-20 C) optionally containing an ether (-0-) linkage, aryl, alkenyl, cycloalkyl, cycloalkenyl or araldyl. Not all linkages in a polynucleotide need be identical. A polynucleotide can contain one or more different types of modifications as described herein and/or multiple modifications of the same type. The preceding description applies to all polynucleotides referred to herein, including RNA and DNA.

“Oligonucleotide,” as used herein, generally refers to short, single stranded, polynucleotides that are, but not necessarily, less than about 250 nucleotides in length. Oligonucleotides may be synthetic. The terms “oligonucleotide” and “polynucleotide” are not mutually exclusive. The description above for polynucleotides is equally and fully applicable to oligonucleotides.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “isolated” antibody is one which has been identified and separated and/or recovered from a component of its natural environment. Contaminant components of its natural environment are materials which would interfere with research, diagnostic, and/or therapeutic uses for the antibody, and may include enzymes, hormones, and other proteinaceous or nonproteinaceous solutes. In some embodiments, an antibody is purified (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of, for example, a spinning cup sequenator, or (3) to homogeneity by SDS-PAGE under reducing or nonreducing conditions using, for example, Coomassie blue or silver stain. An isolated antibody includes the antibody in situ within recombinant cells since at least one component of the antibody's natural environment will not be present. Ordinarily, however, an isolated antibody will be prepared by at least one purification step.

“Native antibodies” are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

The “light chains” of antibodies (immunoglobulins) from any mammalian species can be assigned to one of two clearly distinct types, called kappa (“K”) and lambda (“X”), based on the amino acid sequences of their constant domains.

The term “constant domain” refers to the portion of an immunoglobulin molecule having a more conserved amino acid sequence relative to the other portion of the immunoglobulin, the variable domain, which contains the antigen binding site. The constant domain contains the CH1, CH2, and CH3 domains (collectively, CH) of the heavy chain and the CHL (or CL) domain of the light chain.

The “variable region” or “variable domain” of an antibody refers to the amino-terminal domains of the heavy or light chain of the antibody. The variable domain of the heavy chain may be referred to as “VH.” The variable domain of the light chain may be referred to as “VL.” These domains are generally the most variable parts of an antibody and contain the antigen-binding sites.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions (HVRs) both in the light chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Proteins of Immunological Interest, Fifth Edition, National Institute of Health, Bethesda, Md. (1991)). The constant domains are not involved directly in the binding of an antibody to an antigen, but exhibit various effector functions, such as participation of the antibody in antibody-dependent cellular toxicity.

The term “hypervariable region,” “HVR,” or “HV,” as used herein, refers to the regions of an antibody variable domain which are hypervariable in sequence and/or form structurally defined loops. Generally, antibodies comprise six HVRs; three in the VH (H1, H2, H3), and three in the VL (L1, L2, L3). In native antibodies, H3 and L3 display the most diversity of the six HVRs, and H3 in particular is believed to play a unique role in conferring fine specificity to antibodies. See, for example, Xu et al., Immunity 13:37-45 (2000); Johnson and Wu, in Methods in Molecular Biology 248:1-25 (Lo, ed., Human Press, Totowa, N.J., 2003). Indeed, naturally occurring camelid antibodies consisting of a heavy chain only are functional and stable in the absence of light chain. See, for example, Hamers-Casterman et al., Nature 363:446-448 (1993); Sheriff et al., Nature Struct. Biol. 3:733-736 (1996).

A number of HVR delineations are in use and are encompassed herein. The Kabat Complementarity Determining Regions (CDRs) are based on sequence variability and are the most commonly used (Kabat et al., Sequences of Proteins of Immunological Interest, 5th Ed. Public Health Service, National Institutes of Health, Bethesda, Md. (1991)). Chothia refers instead to the location of the structural loops (Chothia and Lesk J. Mol. Biol. 196:901-917 (1987)). The AbM HVRs represent a compromise between the Kabat HVRs and Chothia structural loops, and are used by Oxford Molecular's AbM antibody modeling software. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2) and 89-97 or 89-96 (L3) in the VL and 26-35 (H1), 50-65 or 49-65 (H2) and 93-102, 94-102, or 95-102 (H3) in the VH. The variable domain residues are numbered according to Kabat et al., supra, for each of these definitions.

“Framework” or “FR” residues are those variable domain residues other than the HVR residues as herein defined.

The term “variable domain residue numbering as in Kabat” or “amino acid position numbering as in Kabat,” and variations thereof, refers to the numbering system used for heavy chain variable domains or light chain variable domains of the compilation of antibodies in Kabat et al., supra. Using this numbering system, the actual linear amino acid sequence may contain fewer or additional amino acids corresponding to a shortening of, or insertion into, a FR or HVR of the variable domain. For example, a heavy chain variable domain may include a single amino acid insert (residue 52a according to Kabat) after residue 52 of H2 and inserted residues (e.g., residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

The Kabat numbering system is generally used when referring to a residue in the variable domain (approximately residues 1-107 of the light chain and residues 1-113 of the heavy chain) (e.g., Kabat et al., Sequences of Immunological Interest. 5th Ed. Public Health Service, National Institutes of Health, Bethesda, Md. (1991)). The “EU numbering system” or “EU index” is generally used when referring to a residue in an immunoglobulin heavy chain constant region (e.g., the EU index reported in Kabat et al., supra). The “EU index as in Kabat” refers to the residue numbering of the human lgG1 EU antibody.

The terms “full-length antibody,” “intact antibody,” and “whole antibody” are used herein interchangeably to refer to an antibody in its substantially intact form, not antibody fragments as defined below. The terms particularly refer to an antibody with heavy chains that contain an Fc region.

“Antibody fragments” comprise a portion of an intact antibody comprising the antigen-binding region thereof. In some embodiments, the antibody fragment described herein is an antigen-binding fragment. Examples of antibody fragments include Fab, Fab′, F(ab′)2, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, e.g., the individual antibodies comprising the population are identical except for possible mutations, e.g., naturally occurring mutations, that may be present in minor amounts. Thus, the modifier “monoclonal” indicates the character of the antibody as not being a mixture of discrete antibodies. In certain embodiments, such a monoclonal antibody typically includes an antibody comprising a polypeptide sequence that binds a target, wherein the target-binding polypeptide sequence was obtained by a process that includes the selection of a single target-binding polypeptide sequence from a plurality of polypeptide sequences. For example, the selection process can be the selection of a unique clone from a plurality of clones, such as a pool of hybridoma clones, phage clones, or recombinant DNA clones. It should be understood that a selected target-binding sequence can be further altered, for example, to improve affinity for the target, to humanize the target-binding sequence, to improve its production in cell culture, to reduce its immunogenicity in vivo, to create a multispecific antibody, etc., and that an antibody comprising the altered target-binding sequence is also a monoclonal antibody of this invention. In contrast to polyclonal antibody preparations, which typically include different antibodies directed against different determinants (epitopes), each monoclonal antibody of a monoclonal antibody preparation is directed against a single determinant on an antigen. In addition to their specificity, monoclonal antibody preparations are advantageous in that they are typically uncontaminated by other immunoglobulins.

The modifier “monoclonal” indicates the character of the antibody as being obtained from a substantially homogeneous population of antibodies, and is not to be construed as requiring production of the antibody by any particular method. For example, the monoclonal antibodies to be used in accordance with the invention may be made by a variety of techniques, including, for example, the hybridoma method (e.g., Kohler and Milstein, Nature 256:495-97 (1975); Hongo et al., Hybridoma 14 (3): 253-260 (1995), Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981)), recombinant DNA methods (see, e.g., U.S. Pat. No. 4,816,567), phage-display technologies (see, e.g., Clackson et al., Nature, 352: 624-628 (1991); Marks et al., J. Mol. Biol. 222: 581-597 (1992); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101 (34): 12467-12472 (2004); and Lee et al., J. Immunol. Methods 284(1-2): 119-132 (2004)), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA 90: 2551 (1993); Jakobovits et al., Nature 362: 255-258 (1993); Bruggemann et al., Year in Immunol. 7:33 (1993); U.S. Pat. Nos. 5,545,807; 5,545,806; 5,569,825; 5,625,126; 5,633,425; and 5,661,016; Marks et al., Bio/Technology 10: 779-783 (1992); Lonberg et al., Nature 368: 856-859 (1994); Morrison, Nature 368: 812-813 (1994); Fishwild et al., Nature Biotechnol. 14: 845-851 (1996); Neuberger, Nature Biotechnol. 14: 826 (1996); and Lonberg et al., Intern. Rev. Immunol. 13: 65-93 (1995)).

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “humanized” antibody refers to a chimeric antibody comprising amino acid residues from non-human HVRs and amino acid residues from human framework regions (FRs). In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody.

A “humanized form” of an antibody, e.g., a non-human antibody, refers to an antibody that has undergone humanization.

A “blocking” antibody or an “antagonist” antibody is one which inhibits or reduces biological activity of the antigen it binds. For example, blocking antibodies or antagonist antibodies substantially or completely inhibit the biological activity of the antigen.

As used herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of <1 μM, <100 nM, <10 nM, <1 nM, or <0.1 nM. In certain embodiments, an antibody specifically binds to an epitope on a protein that is conserved among the protein from different species. In another embodiment, specific binding can include, but does not require exclusive binding.

“Percent (%) amino acid sequence identity” with respect to the polypeptide sequences identified herein is defined as the percentage of amino acid residues in a candidate sequence that are identical with the amino acid residues in the polypeptide being compared, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full-length of the sequences being compared. For purposes herein, however, % amino acid sequence identity values are generated using the sequence comparison computer program ALIGN-2. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, California. The ALIGN-2 program should be compiled for use on a UNIX operating system, for example, digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “detection” includes any means of detecting, including direct and indirect detection. The term “biomarker” as used herein (e.g., a “biomarker” such as LOH of one or more HLA-I genes, or LOH of one or more HLA-I genes and high TMB) refers to an indicator, e.g., predictive, diagnostic, and/or prognostic, which can be detected in a sample. The biomarker may serve as an indicator of a particular subtype of a disease or disorder (e.g., cancer) characterized by certain, molecular, pathological, histological, and/or clinical features (e.g., responsiveness to therapy including a checkpoint inhibitor). In some embodiments, a biomarker is a collection of genes or a collective number of mutations/alterations (e.g., somatic mutations) in a collection of genes. Biomarkers include, but are not limited to, polynucleotides (e.g., DNA and/or RNA), polynucleotide alterations (e.g., polynucleotide copy number alterations, e.g., DNA copy number alterations), polypeptides, polypeptide and polynucleotide modifications (e.g., post-translational modifications), carbohydrates, and/or glycolipid-based molecular markers.

The “amount” or “number” of somatic mutations associated with an increased clinical benefit to an individual is a detectable level in a biological sample. These can be measured by methods known to one skilled in the art and also disclosed herein. The amount of a somatic mutation assessed can be used to determine the response to the treatment.

“Amplification,” as used herein generally refers to the process of producing multiple copies of a desired sequence. “Multiple copies” mean at least two copies. A “copy” does not necessarily mean perfect sequence complementarity or identity to the template sequence. For example, copies can include nucleotide analogs such as deoxyinosine, intentional sequence alterations (such as sequence alterations introduced through a primer comprising a sequence that is hybridizable, but not complementary, to the template), and/or sequence errors that occur during amplification.

The technique of “polymerase chain reaction” or “PCR” as used herein generally refers to a procedure wherein minute amounts of a specific piece of nucleic acid, RNA and/or DNA, are amplified as described, for example, in U.S. Pat. No. 4,683,195. Generally, sequence information from the ends of the region of interest or beyond needs to be available, such that oligonucleotide primers can be designed; these primers will be identical or similar in sequence to opposite strands of the template to be amplified. The 5′ terminal nucleotides of the two primers may coincide with the ends of the amplified material. PCR can be used to amplify specific RNA sequences, specific DNA sequences from total genomic DNA, and cDNA transcribed from total cellular RNA, bacteriophage, or plasmid sequences, etc. See generally Mullis et al., Cold Spring Harbor Symp. Quant. Biol. 51:263 (1987) and Erlich, ed., PCR Technology (Stockton Press, NY, 1989). As used herein, PCR is considered to be one, but not the only, example of a nucleic acid polymerase reaction method for amplifying a nucleic acid test sample, comprising the use of a known nucleic acid (DNA or RNA) as a primer and utilizes a nucleic acid polymerase to amplify or generate a specific piece of nucleic acid or to amplify or generate a specific piece of nucleic acid which is complementary to a particular nucleic acid.

The term “diagnosis” is used herein to refer to the identification or classification of a molecular or pathological state, disease or condition (e.g., cancer). For example, “diagnosis” may refer to identification of a particular type of cancer. “Diagnosis” may also refer to the classification of a particular subtype of cancer, for instance, by histopathological criteria, or by molecular features (e.g., a subtype characterized by expression of one or a combination of biomarkers (e.g., particular genes or proteins encoded by said genes)).

The term “aiding diagnosis” is used herein to refer to methods that assist in making a clinical determination regarding the presence, or nature, of a particular type of symptom or condition of a disease or disorder (e.g., cancer). For example, a method of aiding diagnosis of a disease or condition (e.g., cancer) can comprise measuring certain somatic mutations in a biological sample from an individual.

The term “sample,” as used herein, refers to a composition that is obtained or derived from a subject and/or individual of interest that contains a cellular and/or other molecular entity that is to be characterized and/or identified, for example, based on physical, biochemical, chemical, and/or physiological characteristics. For example, the phrase “disease sample” and variations thereof refers to any sample obtained from a subject of interest that would be expected or is known to contain the cellular and/or molecular entity that is to be characterized. Samples include, but are not limited to, tissue samples, primary or cultured cells or cell lines, cell supernatants, cell lysates, platelets, serum, plasma, vitreous fluid, lymph fluid, synovial fluid, follicular fluid, seminal fluid, amniotic fluid, milk, whole blood, plasma, serum, blood-derived cells, urine, cerebro-spinal fluid, saliva, sputum, tears, perspiration, mucus, tumor lysates, and tissue culture medium, tissue extracts such as homogenized tissue, tumor tissue, cellular extracts, and combinations thereof. In some instances, the sample is a whole blood sample, a plasma sample, a serum sample, or a combination thereof. In some embodiments, the sample is from a tumor (e.g., a “tumor sample”), such as from a biopsy. In some embodiments, the sample is a formalin-fixed paraffin-embedded (FFPE) sample.

A “tumor cell” as used herein, refers to any tumor cell present in a tumor or a sample thereof. Tumor cells may be distinguished from other cells that may be present in a tumor sample, for example, stromal cells and tumor-infiltrating immune cells, using methods known in the art and/or described herein.

A “reference sample,” “reference cell,” “reference tissue,” “control sample,” “control cell,” or “control tissue,” as used herein, refers to a sample, cell, tissue, standard, or level that is used for comparison purposes.

By “correlate” or “correlating” is meant comparing, in any way, the performance and/or results of a first analysis or protocol with the performance and/or results of a second analysis or protocol. For example, one may use the results of a first analysis or protocol in carrying out a second protocol and/or one may use the results of a first analysis or protocol to determine whether a second analysis or protocol should be performed. With respect to the embodiment of polypeptide analysis or protocol, one may use the results of the polypeptide expression analysis or protocol to determine whether a specific therapeutic regimen should be performed. With respect to the embodiment of polynucleotide analysis or protocol, one may use the results of the polynucleotide expression analysis or protocol to determine whether a specific therapeutic regimen should be performed.

“Individual response” or “response” can be assessed using any endpoint indicating a benefit to the individual, including, without limitation, (1) inhibition, to some extent, of disease progression (e.g., cancer progression), including slowing down or complete arrest; (2) a reduction in tumor size; (3) inhibition (i.e., reduction, slowing down, or complete stopping) of cancer cell infiltration into adjacent peripheral organs and/or tissues; (4) inhibition (i.e. reduction, slowing down, or complete stopping) of metastasis; (5) relief, to some extent, of one or more symptoms associated with the disease or disorder (e.g., cancer); (6) increase or extension in the length of survival, including overall survival and progression free survival; and/or (7) decreased mortality at a given point of time following treatment.

An “effective response” of a patient or a patient's “responsiveness” to treatment with a medicament and similar wording refers to the clinical or therapeutic benefit imparted to a patient at risk for, or suffering from, a disease or disorder, such as cancer. In one embodiment, such benefit includes any one or more of: extending survival (including overall survival and/or progression-free survival); resulting in an objective response (including a complete response or a partial response); or improving signs or symptoms of cancer.

An “effective amount” refers to an amount of a therapeutic agent to treat or prevent a disease or disorder in a mammal. In the case of cancers, the therapeutically effective amount of the therapeutic agent may reduce the number of cancer cells; reduce the primary tumor size; inhibit (i.e., slow to some extent and in some embodiments stop) cancer cell infiltration into peripheral organs; inhibit (i.e., slow to some extent and in some embodiments stop) tumor metastasis; inhibit, to some extent, tumor growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), response rates (e.g., CR and PR), duration of response, and/or quality of life.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

As used herein, the terms “individual,” “patient,” or “subject” are used interchangeably and refer to any single animal, e.g., a mammal (including such non-human animals as, for example, dogs, cats, horses, rabbits, zoo animals, cows, pigs, sheep, and non-human primates) for which treatment is desired. In particular embodiments, the patient herein is a human.

As used herein, “administering” is meant a method of giving a dosage of a compound (e.g., an antagonist) or a pharmaceutical composition (e.g., a pharmaceutical composition including an antagonist) to a subject (e.g., a patient). Administering can be by any suitable means, including parenteral, intrapulmonary, and intranasal, and, if desired for local treatment, intralesional administration. Parenteral infusions include, for example, intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

The term “concurrently” is used herein to refer to administration of two or more therapeutic agents, where at least part of the administration overlaps in time. Accordingly, concurrent administration includes a dosing regimen when the administration of one or more agent(s) continues after discontinuing the administration of one or more other agent(s).

The term “package insert” is used to refer to instructions customarily included in commercial packages of therapeutic products, that contain information about the indications, usage, dosage, administration, combination therapy, contraindications, and/or warnings concerning the use of such therapeutic products.

An “article of manufacture” is any manufacture (e.g., a package or container) or kit comprising at least one reagent, e.g., a medicament for treatment of a disease or disorder (e.g., cancer), or a probe for specifically detecting a biomarker (e.g., LOH of one or more HLA-I genes, or LOH of one or more HLA-I genes and high TMB) described herein. In certain embodiments, the manufacture or kit is promoted, distributed, or sold as a unit for performing the methods described herein.

The phrase “based on” when used herein means that the information about one or more biomarkers (e.g., LOH of one or more HLA-I genes, or LOH of one or more HLA-I genes and high TMB) is used to inform a treatment decision, information provided on a package insert, or marketing/promotional guidance, etc.

### III. METHODS, SYSTEMS, AND DEVICES

In one aspect, provided herein are methods of identifying an individual having a squamous cell cancer or NSCLC who may benefit from a treatment comprising an immune checkpoint inhibitor. In some embodiments, the methods comprise detecting in a sample from the individual a somatic loss of heterozygosity (LOH) of one or more human leukocyte antigen class I (HLA-I) genes. In some embodiments, the methods comprise detecting in a sample from the individual a somatic LOH of one or more HLA-I genes and a high tumor mutational burden (TMB). In some embodiments, detection in the sample of the somatic LOH of one or more HLA-I genes, or of the somatic LOH of one or more HLA-I genes and high TMB, identifies the individual as one who may benefit from a treatment comprising an immune checkpoint inhibitor.

In another aspect, provided herein are methods of detecting the presence or absence of a squamous cell cancer or NSCLC in an individual. In some embodiments, the methods comprise detecting the presence or absence of a squamous cell cancer or NSCLC in a sample from the individual. In some embodiments, the methods comprise also detecting in the sample the presence or absence of a somatic LOH of one or more HLA-I genes. In some embodiments, the methods comprise also detecting in the sample the presence or absence of a somatic LOH of one or more HLA-I genes and a high TMB.

In another aspect, provided herein are methods of selecting a therapy for an individual having a squamous cell cancer or NSCLC. In some embodiments, the methods comprise detecting in a sample from the individual a somatic LOH of one or more HLA-I genes. In some embodiments, the methods comprise detecting in a sample from the individual a somatic LOH of one or more HLA-I genes and a high TMB. In some embodiments, detection in the sample of the somatic LOH of one or more HLA-I genes, or of the somatic LOH of one or more HLA-I genes and high TMB, identifies the individual as one who may benefit from a treatment comprising an immune checkpoint inhibitor.

In another aspect, provided herein are methods of identifying one or more treatment options for an individual having a squamous cell cancer or NSCLC. In some embodiments, the methods comprise detecting in a sample from the individual a somatic LOH of one or more HLA-I genes. In some embodiments, the methods comprise detecting in a sample from the individual a somatic LOH of one or more HLA-I genes and a high TMB. In some embodiments, the methods comprise generating a report comprising one or more treatment options identified for the individual based at least in part on the detection in the sample of the somatic LOH of one or more HLA-I genes, or of the somatic LOH of one or more HLA-I genes and high TMB, wherein the one or more treatment options comprise an immune checkpoint inhibitor.

In another aspect, provided herein are methods of identifying one or more treatment options for an individual having a squamous cell cancer or NSCLC. In some embodiments, the methods comprise acquiring knowledge of a somatic LOH of one or more HLA-I genes in a sample from the individual. In some embodiments, the methods comprise acquiring knowledge of a somatic LOH of one or more HLA-I genes and a high TMB in a sample from the individual. In some embodiments, the methods comprise generating a report comprising one or more treatment options identified for the individual based at least in part on said knowledge, wherein the one or more treatment options comprise an immune checkpoint inhibitor.

In another aspect, provided herein are methods of selecting a treatment for an individual having a squamous cell cancer or NSCLC. In some embodiments, the methods comprise acquiring knowledge of a somatic LOH of one or more HLA-I genes in a sample from the individual. In some embodiments, the methods comprise acquiring knowledge of a somatic LOH of one or more HLA-I genes and a high TMB in a sample from the individual. In some embodiments, responsive to the acquisition of said knowledge the individual is classified as a candidate to receive a treatment comprising an immune checkpoint inhibitor. In some embodiments, responsive to the acquisition of said knowledge the individual is identified as likely to respond to a treatment that comprises an immune checkpoint inhibitor.

In another aspect, provided herein are methods of predicting survival of an individual having a squamous cell cancer or NSCLC. In some embodiments, the methods comprise acquiring knowledge of a somatic LOH of one or more HLA-I genes in a sample from the individual. In some embodiments, the methods comprise acquiring knowledge of a somatic LOH of one or more HLA-I genes and a high TMB in a sample from the individual. In some embodiments, responsive to the acquisition of said knowledge, the individual is predicted to have longer survival when treated with a treatment comprising an immune checkpoint inhibitor, as compared to survival of an individual whose squamous cell cancer or NSCLC does not comprise the somatic LOH of one or more HLA-I genes, or the somatic LOH of one or more HLA-I genes and high TMB.

In another aspect, provided herein are methods of predicting survival of an individual having a squamous cell cancer or NSCLC treated with an immune checkpoint inhibitor. In some embodiments, the methods comprise acquiring knowledge of a somatic LOH of one or more HLA-I genes in a sample from the individual. In some embodiments, the methods comprise acquiring knowledge of a somatic LOH of one or more HLA-I genes and a high TMB in a sample from the individual. In some embodiments, responsive to the acquisition of said knowledge, the individual is predicted to have longer survival after a treatment comprising an immune checkpoint inhibitor, as compared to an individual whose cancer does not exhibit the somatic LOH of one or more HLA-I genes, or the somatic LOH of one or more HLA-I genes and high TMB.

In another aspect, provided herein are methods of treating or delaying progression of a squamous cell cancer or NSCLC. In some embodiments, the methods comprise acquiring knowledge of a somatic LOH of one or more HLA-I genes in a sample from an individual having a squamous cell cancer or NSCLC. In some embodiments, the methods comprise acquiring knowledge of a somatic LOH of one or more HLA-I genes and a high TMB in a sample from an individual having a squamous cell cancer or NSCLC. In some embodiments, the methods comprise, responsive to said knowledge, administering to the individual an effective amount of a treatment that comprises an immune checkpoint inhibitor.

In another aspect, provided herein are methods of treating or delaying progression of a squamous cell cancer or NSCLC. In some embodiments, the methods comprise, responsive to acquiring knowledge of a somatic LOH of one or more HLA-I genes in a sample from an individual having a squamous cell cancer or NSCLC, administering to the individual an effective amount of a treatment that comprises an immune checkpoint inhibitor. In some embodiments, the methods comprise, responsive to acquiring knowledge of a somatic LOH of one or more HLA-I genes and a high TMB in a sample from an individual having a squamous cell cancer or NSCLC, administering to the individual an effective amount of a treatment that comprises an immune checkpoint inhibitor.

In another aspect, provided herein are methods of monitoring, evaluating, or screening an individual having a squamous cell cancer or NSCLC. In some embodiments, the methods comprise acquiring knowledge of a somatic LOH of one or more HLA-I genes in a sample from the individual. In some embodiments, the methods comprise acquiring knowledge of a somatic LOH of one or more HLA-I genes and a high TMB in a sample from the individual. In some embodiments, responsive to the acquisition of said knowledge, the individual is predicted to have longer survival when treated with a treatment comprising an immune checkpoint inhibitor, as compared to an individual whose squamous cell cancer or NSCLC does not comprise the somatic LOH of one or more HLA-I genes, or the somatic LOH of one or more HLA-I genes and high TMB.

In another aspect, provided herein are methods of treating or delaying progression of a squamous cell cancer or NSCLC. In some embodiments, the methods comprise detecting in a sample from an individual having a squamous cell cancer or NSCLC a somatic LOH of one or more HLA-I genes. In some embodiments, the methods comprise detecting in a sample from an individual having a squamous cell cancer or NSCLC a somatic LOH of one or more HLA-I genes and a high TMB. In some embodiments, the methods comprise administering to the individual an effective amount of a treatment that comprises an immune checkpoint inhibitor.

In another aspect, provided herein are methods of assessing a squamous cell cancer or NSCLC in an individual. In some embodiments, the methods comprise detecting in a sample from the individual a somatic LOH of one or more HLA-I genes. In some embodiments, the methods comprise detecting in a sample from the individual a somatic LOH of one or more HLA-I genes and a high TMB. In some embodiments, the methods comprise providing an assessment of the somatic LOH of one or more HLA-I genes, or the somatic LOH of one or more HLA-I genes and high TMB, in the squamous cell cancer or NSCLC.

In another aspect, provided herein are methods of treating or delaying progression of a squamous cell cancer or NSCLC. In some embodiments, the methods comprise administering to an individual having a squamous cell cancer or NSCLC an effective amount of a treatment comprising an immune checkpoint inhibitor, wherein the squamous cell cancer or NSCLC comprises a somatic LOH of one or more HLA-I genes. In some embodiments, the methods comprise administering to an individual having a squamous cell cancer or NSCLC an effective amount of a treatment comprising an immune checkpoint inhibitor, wherein the squamous cell cancer or NSCLC comprises a somatic LOH of one or more HLA-I genes and high TMB.

In another aspect, provided herein are systems comprising a memory configured to store one or more program instructions; and one or more processors configured to execute the one or more program instructions. In some embodiments, the one or more program instructions when executed by the one or more processors are configured to: (a) obtain a plurality of sequence reads of one or more nucleic acids, wherein the one or more nucleic acids are derived from a sample of a squamous cell cancer or NSCLC obtained from an individual; (b) analyze the plurality of sequence reads for the presence of a somatic LOH of one or more HLA-I genes, or a somatic LOH of one or more HLA-I genes and a high TMB; (c) detect, based on the analyzing, the presence of a somatic LOH of one or more HLA-I genes, or a somatic LOH of one or more HLA-I genes and high TMB, in the sample; and (d) generating, based at least in part on the detecting, a genomic profile for the sample. In some embodiments, a genomic profile comprises the presence or absence of one or more genomic alterations in the sample, e.g., LOH of one or more HLA-I genes, or LOH of one or more HLA-I genes and a high TMB. In some embodiments, the genomic profile indicates the presence or absence of one or more genomic alterations in the sample, e.g., LOH of one or more HLA-I genes, or LOH of one or more HLA-I genes and a high TMB. In some embodiments, the genomic profile comprises information on the presence or absence of one or more genomic alterations in the sample, e.g., LOH of one or more HLA-I genes, or LOH of one or more HLA-I genes and a high TMB.

In another aspect, provided herein are non-transitory computer readable storage media. In some embodiments, the non-transitory computer readable storage media comprise one or more programs executable by one or more computer processors for performing a method. In some embodiments, the method comprises: (a) obtaining, using the one or more processors, a plurality of sequence reads of one or more nucleic acids, wherein the one or more nucleic acids are derived from a sample of a squamous cell cancer or NSCLC obtained from an individual; (b) analyzing, using the one or more processors, the plurality of sequence reads for the presence of a somatic LOH of one or more HLA-I genes, or a somatic LOH of one or more HLA-I genes and a high TMB; (c) detecting, using the one or more processors and based on the analyzing, the presence of a somatic LOH of one or more HLA-I genes, or of a somatic LOH of one or more HLA-I genes and high TMB, in the sample; and (d) generating, based at least in part on the detecting, a genomic profile for the sample. In some embodiments, a genomic profile comprises the presence or absence of one or more genomic alterations in the sample, e.g., LOH of one or more HLA-I genes, or LOH of one or more HLA-I genes and a high TMB. In some embodiments, the genomic profile indicates the presence or absence of one or more genomic alterations in the sample, e.g., LOH of one or more HLA-I genes, or LOH of one or more HLA-I genes and a high TMB. In some embodiments, the genomic profile comprises information on the presence or absence of one or more genomic alterations in the sample, e.g., LOH of one or more HLA-I genes, or LOH of one or more HLA-I genes and a high TMB.

### Cancers

Certain aspects of the present disclosure are related to methods of detecting LOH of one or more HLA-I genes and/or high TMB in squamous cell cancer or non-small cell lung cancer (NSCLC), as well as methods of treating, assessing, diagnosing, monitoring, evaluating or screening individuals having a squamous cell cancer or NSCLC.

### Squamous Cell Cancer

Squamous cell cancer, also known as squamous cell carcinoma (SCC) or epidermoid carcinoma, is a type of cancer that arises from squamous cells, which are found on the surface of skin, and the lining of hollow organs and respiratory and digestive tracts. Common types of SCC include squamous cell skin cancer, squamous cell carcinoma of the lung, squamous cell thyroid carcinoma, esophageal squamous cell carcinoma, and squamous cell carcinoma of the vagina. Diagnosis of squamous cell cancer may involve a tumor biopsy and/or histopathology. TP63 staining is a common histological marker for squamous cell carcinoma. Subtypes of squamous cell cancers include papillary thyroid carcinoma, verrucous squamous cell carcinoma, papillary squamous cell carcinoma, squamous cell carcinoma, large-cell keratinizing squamous cell carcinoma, large-cell nonkeratinizing squamous cell carcinoma, small-cell keratinizing squamous cell carcinoma, spindle-cell squamous cell carcinoma, spindle-cell carcinoma, adenoid/pseudoglandular squamous cell carcinoma, intraepidermal squamous cell carcinoma, lymphoepithelial carcinoma, keratoacanthoma, Erythroplasia of Queyrat, Marjolin's ulcer, adenoid squamous cell carcinoma, basaloid squamous cell carcinoma, clear-cell squamous cell carcinoma, or signet ring-cell squamous cell carcinoma.

In some embodiments, a squamous cell cancer of the disclosure is a skin, lip, mouth, esophageal, head and neck, urinary tract, prostate, lung, vaginal, or cervical cancer. In some embodiments, a squamous cell cancer of the disclosure is a squamous cell skin cancer, squamous cell carcinoma of the lung, squamous cell thyroid carcinoma, esophageal squamous cell carcinoma, or squamous cell carcinoma of the vagina. In some embodiments, a squamous cell cancer of the disclosure is a non-melanoma skin cancer. In some embodiments, a squamous cell cancer of the disclosure is a head and neck cancer. In some embodiments, a squamous cell cancer of the disclosure is an esophageal cancer. In some embodiments, a squamous cell cancer of the disclosure is a squamous cell lung cancer. In some embodiments, the squamous cell lung cancer is a non-small cell lung cancer (NSCLC). In some embodiments, a squamous cell cancer of the disclosure is of any subtype known in the art or described herein.

In some embodiments, a squamous cell cancer of the disclosure has a somatic loss of heterozygosity (LOH) of one or more human leukocyte antigen class I (HLA-I) genes, e.g., one or more of HLA-A, HLA-B or HLA-C. In some embodiments, a squamous cell cancer of the disclosure has a high tumor mutational burden (TMB), e.g., of at least about 10 mutations/megabase or at least about 13 mutations/Mb. In some embodiments, a squamous cell lung cancer of the disclosure has LOH of one or more HLA-I genes and a high TMB, e.g., of at least about 10 mutations/megabase. LOH of one or more HLA-I genes and TMB may be assessed using any method known in the art, e.g., as described below.

In some embodiments, cancer of the disclosure (e.g., a squamous cell cancer described herein) is PD-L1 positive. PD-L1 positivity may be assessed according to any method known in the art, e.g., as described below.

In some embodiments, the squamous cell cancer does not comprise a mutation or genomic alteration in a human EGFR gene and/or a human ALK gene. In some embodiments, the squamous cell cancer is EGFR-wild type and/or ALK-wild type. In some embodiments, the squamous cell cancer does not comprise a pathogenic mutation or genomic alteration in a human EGFR gene and/or a human ALK gene. Mutations or genomic alterations (e.g., in EGFR or ALK) may be assessed using any suitable method known in the art, including but not limited to, a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing nucleic acid samples, e.g., to detect a mutation or genomic alteration, are described in U.S. Pat. No. 9,340,830; WO2012092426A1; Frampton, G. M. et al. (2013) Nat. Biotech. 31:1023-1031; and Montesion, M., et al., Cancer Discovery (2021) 11(2):282-92, the entirety of each which is incorporated by reference herein. In some embodiments, the mutation or genomic alteration is detected in an encoded polypeptide or protein. Any suitable method known in the art may be used, including without limitation, immunoblotting, enzyme linked immunosorbent assay (ELISA), immunohistochemistry, or mass spectrometry.

In some embodiments, the squamous cell cancer is an advanced squamous cell cancer.

In some embodiments, the squamous cell cancer is a metastatic squamous cell cancer.

In some embodiments, the squamous cell cancer was previously treated with an immune checkpoint inhibitor, e.g., an immune checkpoint inhibitor described herein. In some embodiments, the squamous cell cancer was previously treated with an anti-cancer therapy other than an immune checkpoint inhibitor, e.g., an anti-cancer therapy described herein. In some embodiments, the squamous cell cancer was not previously treated with an immune checkpoint inhibitor. In some embodiments, the squamous cell cancer was not previously treated with an anti-cancer therapy other than an immune checkpoint inhibitor. In some embodiments, the squamous cell cancer was not previously treated. In some embodiments, the squamous cell cancer was previously treated with a first line anti-cancer therapy for squamous cell cancer, e.g., an anti-cancer therapy described herein. In some embodiments, the squamous cell cancer was previously treated with a second line anti-cancer therapy for squamous cell cancer, e.g., an anti-cancer therapy described herein. In some embodiments, the squamous cell cancer was previously treated with a first line immune checkpoint inhibitor for squamous cell cancer, e.g., an immune checkpoint inhibitor described herein. In some embodiments, the squamous cell cancer was previously treated with a second line immune checkpoint inhibitor for squamous cell cancer, e.g., an immune checkpoint inhibitor described herein. In some embodiments, the first line anti-cancer therapy comprises carboplatin, paclitaxel, paclitaxel protein-bound (e.g., Abraxane), gemcitabine, docetaxel, ramucirumab, or any combination thereof. In some embodiments, the first line anti-cancer therapy comprises carboplatin and paclitaxel; carboplatin and paclitaxel protein-bound (e.g., Abraxane); carboplatin and gemcitabine; or docetaxel and ramucirumab.

### Squamous Cell Lung Cancer

Squamous cell lung cancer, also known as squamous cell carcinoma (SCC) of the lung, is a common type of lung cancer. Squamous cell lung cancer often originates in the bronchi and is characterized by a squamous appearance. Squamous cell lung cancer often metastasizes to loco-regional lymph nodes and can disseminate outside the thorax. Squamous cell lung cancer is generally asymptomatic early during progression of the disease, often being detected as an incidental finding on imaging studies, e.g., computed tomography or magnetic resonance imaging studies. As the disease progresses, it generally becomes symptomatic, e.g., when the tumor mass obstructs structures in the lungs such as a major bronchus. Diagnosis may involve histopathology of lung biopsies, or cytopathology of cytological smear tests of sputum, bronchoalveolar lavage or endobronchial brushings. An important risk factor for squamous cell lung cancer is tobacco smoking.

Squamous cell lung cancers may be classified as keratinizing squamous cell carcinoma, non-keratinizing squamous cell carcinoma, basaloid squamous cell carcinoma, pervasive lesion, and squamous cell carcinoma in situ. See, e.g., Travis et al., Journal of Thoracic Oncology (2015) 10(9): 1243-1260. Squamous cell lung cancers may be staged according to methods known in the art, such as the Tumor-Node-Metastasis (TNM) staging system, e.g., including the occult stage, Stage 0, Stage I (including Stage IA and IB), Stage II (including Stage IIA and IIB), Stage III (including Stage IIIA, IIIB and IIIC), or Stage IV (including Stage IVA and IVB).

In some embodiments, a squamous cell lung cancer of the disclosure has a somatic loss of heterozygosity (LOH) of one or more human leukocyte antigen class I (HLA-I) genes, e.g., one or more of HLA-A, HLA-B or HLA-C. In some embodiments, a squamous cell lung cancer of the disclosure has a high tumor mutational burden (TMB), e.g., of at least about 10 mutations/megabase. In some embodiments, a squamous cell lung cancer of the disclosure has LOH of one or more HLA-I genes and a high TMB, e.g., of at least about 10 mutations/megabase. LOH of one or more HLA-I genes and TMB may be assessed using any method known in the art, e.g., as described below.

In some embodiments, the squamous cell lung cancer does not comprise a mutation or genomic alteration in a human EGFR gene and/or a human ALK gene. In some embodiments, the squamous cell lung cancer is EGFR-wild type and/or ALK-wild type. In some embodiments, the squamous cell lung cancer does not comprise a pathogenic mutation or genomic alteration in a human EGFR gene and/or a human ALK gene. In some embodiments, the squamous cell lung cancer comprises a mutation or a genomic alteration in a human CDKN2A gene, a human SOX2 gene, a human LRP1B gene, a human BRCA1 gene, a human FGF12 gene, a human TERC gene, a human PIK3CA gene, a human PRKCI gene, a human PTEN gene, a human ARID1A gene, a human KDM5A gene, a human SPTA1 gene, a human FAS gene, a human FUBP1 gene, or any combination thereof. In some embodiments, the mutation or genomic alteration comprises one or more of a short variant alteration (e.g., a base substitution, insertion, or deletion), a copy-number alteration (e.g., an amplification or a homozygous deletion), or a rearrangement (e.g., a gene fusion or other genomic or chromosomal rearrangement). In some embodiments, the mutation or the genomic alteration results in a substitution, insertion, or deletion of one or more amino acid residues in a polypeptide or a protein encoded by the gene. Mutations or genomic alterations may be assessed using any suitable method known in the art, including but not limited to, a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing nucleic acid samples, e.g., to detect mutations or genomic alterations, are described in U.S. Pat. No. 9,340,830; WO2012092426A1; Frampton, G. M. et al. (2013) Nat. Biotech. 31:1023-1031; and Montesion, M., et al., Cancer Discovery (2021) 11(2):282-92, the entirety of each which is incorporated by reference herein. In some embodiments, the mutation or genomic alteration is detected in an encoded polypeptide or protein. Any suitable method known in the art may be used, including without limitation, immunoblotting, enzyme linked immunosorbent assay (ELISA), immunohistochemistry, or mass spectrometry.

In some embodiments, the squamous cell lung cancer comprises a tobacco signature. A tobacco signature refers to a set of DNA mutations associated with tobacco smoking, e.g., as is known in the art and/or as identified in the Catalogue of Somatic Mutations in Cancer (COSMIC) mutational signature project, e.g., available at the website: cancer[dot]sanger[dot]ac.uk/signatures/. In some embodiments, a tobacco signature is assessed using any method known in the art, such as the methods described by Zehir et al., Nat Med (2017) 23:703-13.

In some embodiments, the squamous cell lung cancer is PD-L1-positive. PD-L1 positivity may be assessed using any method known in the art, e.g., as described below.

In some embodiments, the squamous cell lung cancer is a keratinizing squamous cell carcinoma. In some embodiments, the squamous cell lung cancer is a non-keratinizing squamous cell carcinoma. In some embodiments, the squamous cell lung cancer is a basaloid squamous cell carcinoma. In some embodiments, the squamous cell lung cancer is a pervasive lesion. In some embodiments, the squamous cell lung cancer is a squamous cell carcinoma in situ. In some embodiments, the squamous cell lung cancer is an occult stage, Stage 0, Stage I, Stage IA, Stage IB, Stage II, Stage IIA, Stage IIB, Stage III, Stage IIIA, Stage IIIB, Stage IIIC, Stage IV, Stage IVA, or Stage IVB squamous cell lung cancer. In some embodiments, the squamous cell lung cancer is an advanced squamous cell lung cancer. In some embodiments, the squamous cell lung cancer is a metastatic squamous cell lung cancer.

In some embodiments, the squamous cell lung cancer is a non-small cell lung cancer (NSCLC). In some embodiments, the NSCLC is a keratinizing NSCLC. In some embodiments, the NSCLC is a non-keratinizing NSCLC. In some embodiments, the NSCLC is a basaloid NSCLC. In some embodiments, the NSCLC is a pervasive lesion. In some embodiments, the NSCLC is a NSCLC in situ. In some embodiments, the NSCLC is an occult stage, Stage 0, Stage I, Stage IA, Stage IB, Stage II, Stage IIA, Stage IIB, Stage III, Stage IIIA, Stage IIIB, Stage IIIC, Stage IV, Stage IVA, or Stage IVB NSCLC. In some embodiments, the NSCLC is advanced NSCLC. In some embodiments, the NSCLC is metastatic NSCLC.

In some embodiments, the squamous cell lung cancer was previously treated with an immune checkpoint inhibitor, e.g., an immune checkpoint inhibitor described herein. In some embodiments, the squamous cell lung cancer was previously treated with an anti-cancer therapy other than an immune checkpoint inhibitor, e.g., an anti-cancer therapy described herein. In some embodiments, the squamous cell lung cancer was not previously treated with an immune checkpoint inhibitor. In some embodiments, the squamous cell lung cancer was not previously treated with an anti-cancer therapy other than an immune checkpoint inhibitor. In some embodiments, the squamous cell lung cancer was not previously treated. In some embodiments, the squamous cell lung cancer was previously treated with a first line anti-cancer therapy for squamous cell lung cancer, e.g., an anti-cancer therapy described herein. In some embodiments, the squamous cell lung cancer was previously treated with a second line anti-cancer therapy for squamous cell lung cancer, e.g., an anti-cancer therapy described herein. In some embodiments, the squamous cell lung cancer was previously treated with a first line immune checkpoint inhibitor for squamous cell lung cancer, e.g., an immune checkpoint inhibitor described herein. In some embodiments, the squamous cell lung cancer was previously treated with a second line immune checkpoint inhibitor for squamous cell lung cancer, e.g., an immune checkpoint inhibitor described herein. In some embodiments, the first line anti-cancer therapy comprises carboplatin, paclitaxel, paclitaxel protein-bound (e.g., Abraxane), gemcitabine, docetaxel, ramucirumab, or any combination thereof. In some embodiments, the first line anti-cancer therapy comprises carboplatin and paclitaxel; carboplatin and paclitaxel protein-bound (e.g., Abraxane); carboplatin and gemcitabine; or docetaxel and ramucirumab.

### Non-Small Cell Lung Cancer

Non-small cell lung cancer (NSCLC), also known as non-small cell lung carcinoma, refers to all epithelial lung cancers other than small cell-lung cancers or small cell-lung carcinomas. Types of NSCLC include squamous cell carcinoma, large-cell carcinoma, adenocarcinoma, pleomorphic NSCLC, carcinoid tumor, adenosquamous cancer, sarcomatoid cancer, salivary gland carcinoma, undifferentiated cancer, and unclassified carcinoma.

In some embodiments, a NSCLC of the disclosure is an adenocarcinoma.

In some embodiments, a NSCLC of the disclosure is a squamous cell NSCLC.

In some embodiments, a NSCLC of the disclosure is of any subtype known in the art or described herein.

In some embodiments, a NSCLC of the disclosure has a somatic loss of heterozygosity (LOH) of one or more human leukocyte antigen class I (HLA-I) genes, e.g., one or more of HLA-A, HLA-B or HLA-C. In some embodiments, a NSCLC of the disclosure has a high tumor mutational burden (TMB), e.g., of at least about 10 mutations/megabase. In some embodiments, a NSCLC of the disclosure has LOH of one or more HLA-I genes and a high TMB, e.g., of at least about 10 mutations/megabase. LOH of one or more HLA-I genes and TMB may be assessed using any method known in the art, e.g., as described below.

In some embodiments, a NSCLC of the disclosure is PD-L1 positive. PD-L1 positivity may be assessed using any method known in the art, e.g., as described below.

In some embodiments, the NSCLC is an advanced NSCLC.

In some embodiments, the NSCLC is a metastatic NSCLC.

In some embodiments, the NSCLC does not comprise a mutation or genomic alteration in a human EGFR gene and/or a human ALK gene. In some embodiments, the NSCLC is EGFR-wild type and/or ALK-wild type. In some embodiments, the NSCLC does not comprise a pathogenic mutation or genomic alteration in a human EGFR gene and/or a human ALK gene. In some embodiments, the NSCLC comprises a mutation or a genomic alteration in a human CDKN2A gene, a human SOX2 gene, a human LRP1B gene, a human BRCA1 gene, a human FGF12 gene, a human TERC gene, a human PIK3CA gene, a human PRKCI gene, a human PTEN gene, a human ARID1A gene, a human KDM5A gene, a human SPTA1 gene, a human FAS gene, a human FUBP1 gene, or any combination thereof. In some embodiments, the mutation or genomic alteration comprises one or more of a short variant alteration (e.g., a base substitution, insertion, or deletion), a copy-number alteration (e.g., an amplification or a homozygous deletion), or a rearrangement (e.g., a gene fusion or other genomic or chromosomal rearrangement). In some embodiments, the mutation or the genomic alteration results in a substitution, insertion, or deletion of one or more amino acid residues in a polypeptide or a protein encoded by the gene. Mutations or genomic alterations may be assessed using any suitable method known in the art, including but not limited to, a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing nucleic acid samples, e.g., to detect mutations or genomic alterations, are described in U.S. Pat. No. 9,340,830; WO2012092426A1; Frampton, G. M. et al. (2013) Nat. Biotech. 31:1023-1031; and Montesion, M., et al., Cancer Discovery (2021) 11(2):282-92, the entirety of each which is incorporated by reference herein. In some embodiments, the mutation or genomic alteration is detected in an encoded polypeptide or protein. Any suitable method known in the art may be used, including without limitation, immunoblotting, enzyme linked immunosorbent assay (ELISA), immunohistochemistry, or mass spectrometry.

In some embodiments, the NSCLC comprises a tobacco signature.

In some embodiments, the NSCLC was previously treated with an immune checkpoint inhibitor, e.g., an immune checkpoint inhibitor described herein. In some embodiments, the NSCLC was previously treated with an anti-cancer therapy other than an immune checkpoint inhibitor, e.g., an anti-cancer therapy described herein. In some embodiments, the NSCLC was not previously treated with an immune checkpoint inhibitor. In some embodiments, the NSCLC was not previously treated with an anti-cancer therapy other than an immune checkpoint inhibitor. In some embodiments, the NSCLC was not previously treated. In some embodiments, the NSCLC was previously treated with a first line anti-cancer therapy for NSCLC, e.g., an anti-cancer therapy described herein. In some embodiments, the NSCLC was previously treated with a second line anti-cancer therapy for NSCLC, e.g., an anti-cancer therapy described herein. In some embodiments, the NSCLC was previously treated with a first line immune checkpoint inhibitor for NSCLC, e.g., an immune checkpoint inhibitor described herein. In some embodiments, the NSCLC was previously treated with a second line immune checkpoint inhibitor for NSCLC, e.g., an immune checkpoint inhibitor described herein. In some embodiments, the first line anti-cancer therapy comprises carboplatin, paclitaxel, paclitaxel protein-bound (e.g., Abraxane), gemcitabine, docetaxel, ramucirumab, or any combination thereof. In some embodiments, the first line anti-cancer therapy comprises carboplatin and paclitaxel; carboplatin and paclitaxel protein-bound (e.g., Abraxane); carboplatin and gemcitabine; or docetaxel and ramucirumab.

### Loss of Heterozygosity of Human Leukocyte Antigen Class I Genes

Immune checkpoint inhibitors (ICIs) function by reinvigorating the immune system and enabling T cell-mediated tumor elimination. This elimination is dependent upon T cell recognition, achieved through presentation of tumor-specific antigens by human leukocyte antigen class I (HLA-I) gene-encoded major histocompatibility complex class I (MHC-I) proteins. The HLA-I genes in humans include the genes HLA-A, HLA-B and HLA-C. Disruption of MHC-I presentation, through mechanisms such as loss of heterozygosity (LOH) of one or more of the HLA-I genes, is one potential mechanism of immune evasion and could affect responses to immune checkpoint inhibitors.

As demonstrated herein, LOH of one or more HLA-I genes in squamous cell cancer or NSCLC, e.g., squamous cell lung cancer or squamous NSCLC, may be predictive of increased overall survival, increased progression-free survival, increased probability of greater survival, and/or increased likelihood of response to immune checkpoint inhibitor therapy, e.g., as compared to squamous cell cancer or NSCLC without LOH of an HLA-I gene. See, e.g., Example 1. Accordingly, in some embodiments, provided herein are methods that comprise acquiring knowledge of or detecting a LOH of one or more HLA-I genes in a sample from a squamous cell cancer or NSCLC obtained from an individual. In some embodiments, a cancer of the disclosure, e.g., a squamous cell cancer or NSCLC, has a somatic LOH of one or more HLA-I genes, e.g., one or more of HLA-A, HLA-B or HLA-C. As used herein, LOH can refer to copy-loss LOH and/or copy-neutral LOH.

### Methods of Detection of Loss of Heterozygosity of Human Leukocyte Antigen Class I Genes

LOH of one or more HLA-I genes, e.g., one or more of HLA-A, HLA-B or HLA-C, may be assessed using any suitable method known in the art, including, without limitation, sequencing (e.g., whole exome sequencing, whole genome sequencing, gene-targeted sequencing, methylation sequencing, or next-generation sequencing) and hybrid-capture-based sequencing methods.

In some embodiments, LOH of one or more HLA-I genes, e.g., one or more of HLA-A, HLA-B or HLA-C, is assessed using a hybrid-capture-based sequencing method.

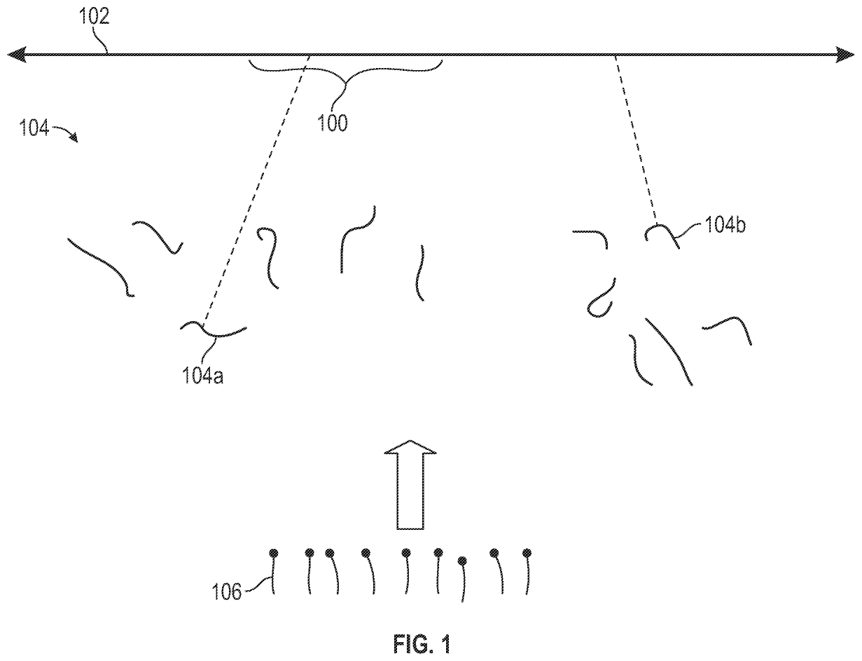

FIG. 1 illustrates a hybrid capture process. Further details about this and other hybrid capture processes can be found in U.S. Pat. No. 9,340,830; Frampton, G. M. et al. (2013) Nat. Biotech. 31:1023-1031; and Montesion, M., et al., Cancer Discovery (2021) 11(2):282-92, the entirety of each which is incorporated by reference herein. A population of DNA fragments 104 from the subject is prepared, some of which correspond to the gene of interest 100 (e.g., an HLA-I gene) within the subject's genome 102. If the subject is heterozygous at the gene of interest 100 then population of DNA fragments 104 will comprise different alleles (one from each parent), in roughly equal amounts. On the other hand, if the subject has undergone LOH, then one of the parent's alleles will be absent or significantly decreased in the population of on-target fragments 104a.

Thus, consistent with the hybrid capture approach, a population of bait molecules 106 corresponding to the gene of interest 100 are introduced to the population of the subject's DNA fragments 104. The bait molecules 106 will bond with “on-target” fragments 104a—that is, DNA fragments 104 that originate from the gene of interest 100. Conversely, the bait molecules 106 will not bond with “off-target” fragments 104b.

After sufficient time to allow such bonding to happen, the fragment/bait hybrids are captured and the remaining fragments are discarded. The captured hybrids are then sequenced to determine which alleles are present, and their relative frequencies. If the allele frequencies are sufficiently close to equal, then the patient can be determined to be heterozygous. If one allele frequency is sufficiently low, then the patient can be determined to have undergone LOH in the gene of interest 100, e.g., an HLA-I gene.

This relatively straightforward process can be complicated by a number of factors. First, the patient sample may be of a mixed nature. For example, if the sample comes from a tumor biopsy, the sample may contain both normal, healthy cells from the patient as well as cancerous cells from a tumor. Second, some cancer cells may exhibit aneuploidy, in which the cancer cells have a greater or lesser than typical number of duplicate chromosomes. If one or both of these factors are present, they may change the expected allele frequencies for either a heterozygous subject or a subject that has experienced LOH.

Consequently, in the hybrid capture process described above, a particular bait molecule 106 will not enjoy perfect complementarity with most possible alleles. Moreover, different alleles may compete with each other to bind with the same bait molecule. In turn, these phenomena can effect (sometimes profoundly) the propensity of an on-target fragment 104a successfully binding to a bait molecule 106. This results in that particular allele being under-sampled or over-sampled by the capture process, and therefore in a measured allele frequency that is artificially high or low, and in some cases incorrectly making a determination whether the subject has experienced LOH or not. These errors can be exacerbated by genes of interest 100 exhibiting a large degree of polymorphism, such as the HLA genes, which can have up to thousands of possible alleles.

Certain approaches have been developed to assess LOH and may be used to detect HLA-I LOH according to any of the methods provided herein, including approaches based or not based on hybrid capture and/or sequencing.

One exemplary approach to assess HLA-I LOH that may be used is the loss of heterozygosity in human leukocyte antigen (LOHHLA) method. The LOHHLA method leverages sequencing reads that map specifically to an individual's germline HLA alleles rather than a human reference genome to determine HLA haplotype-specific copy number and assess HLA LOH. As described in detail in McGranahan et al., Cell (2017) 171(6): 1259-1271.e11, which is incorporated by reference herein, in the LOHHLA method, tumor and germline sequencing reads that map to the HLA region of the genome and chromosome 6 are extracted; reads are aligned to patient-specific HLA alleles, which may be obtained from HLA serotyping or an inference tool, e.g., Polysolver (Shukla et al., Nat. Biotechnol (20150 33:1152-1158.) or Optitype (Szolek et al., Bioinformatics (2014) 30:3310-3316); polymorphic sites between homologous HLA alleles are identified; tumor coverage relative to germline (log R) and b-allele frequencies (BAF) are inferred at each HLA locus, making use of identified polymorphic sites; and HLA allele-specific copy number is determined for each HLA gene, accounting for stromal contamination. The LOHHLA method also incorporates additional parameters, including tumor purity (i.e., the proportion of the sample that contains tumor cells vs. healthy cells) and tumor ploidy (i.e., the number of duplicate chromosomes the tumor cells possess) to determine the HLA allele-specific copy number. In some embodiments, the methods provided herein comprise detecting somatic LOH of one or more HLA-I genes in a squamous cell cancer or NSCLC using the LOHHLA method, e.g., as described in detail in McGranahan et al., Cell (2017) 171(6): 1259-1271.e11. In some embodiments, the methods comprise determining the specific copy number of an HLA allele of the one or more HLA-I genes in the squamous cell cancer or NSCLC. In some embodiments, the methods comprise one or more, or all, of the following steps: (a) aligning a plurality of sequence reads of an HLA allele of one or more HLA-I genes with reference sequence reads of an HLA allele of one or more HLA-I genes, wherein the plurality of sequence reads is derived from a sample of the squamous cell cancer or NSCLC, and wherein the reference sequence reads are based on the individual's HLA type; (b) determining mismatch positions in homologous HLA alleles of the one or more HLA-I genes, and determining mismatch coverage for each HLA allele; (c) determining the ratio and allele frequency of each HLA allele based on mismatches and coverage determined in step (b); and (d) determining the copy number of each HLA allele in the squamous cell cancer or NSCLC based on the ratio and allele frequency determined in step (c). In some embodiments, the plurality of sequence reads is obtained by whole exome sequencing, whole genome sequencing, gene-targeted sequencing, methylation sequencing, or next-generation sequencing. In some embodiments, the plurality of sequence reads is obtained by next-generation sequencing.

Another exemplary approach to assess LOH that may be used mitigates the sampling error that can occur in the hybrid capture process as described above. See, e.g., Montesion, M., et al., Cancer Discovery (2021) 11(2):282-92. In some embodiments, an approach to assess LOH that may be used mitigates the sampling error that can occur in the hybrid capture process by empirically determining relative binding propensities of the various alleles to a particular bait molecule; e.g., if a sample of subject DNA fragments 104 truly included equal proportions of on-target fragments 104a from two different alleles, then it may be empirically determined what actual allele frequencies result from a hybrid capture process using a particular bait molecule 106. Because the binding propensities are due, at least in part, to inter-allelic competition, this determination may be made on an allele-pair-by-allele-pair basis, not just on an allele-by-allele basis. If those relative binding propensities were known, then the sampling bias of subsequent hybrid capture processes with those alleles and bait molecules can be corrected by scaling the observed allele frequencies. For example, an objective function can be applied to measure a difference between the relative binding propensity and the observed allele frequency of a given allele. But for highly polymorphic genes like HLA, this approach may not be practical, insofar as there are too many allele pairs to determine all the relative binding propensities. However, the following techniques can be useful if only a subset of relative binding propensities are known.

In what follows, suppose the gene of interest 100 is a polymorphic gene having n possible alleles. The relative binding propensity of alleles i and j to a bait molecule is given by:

$$\frac{{k}_{i}}{{k}_{j}} = \frac{{AF}_{i}}{{AF}_{j}}$$

Where kᵢ and kⱼ are the relative binding propensities of alleles i and j, respectively, and AFᵢ and AFⱼ represent the corresponding allele frequencies of alleles i and j respectively.

Note that since AFᵢ and AFⱼ are determined in part by the interaction of their corresponding alleles, these numbers should be understood to describe allele frequencies of alleles i and j only in the presence of the other allele. In other words, if i, j, and k are distinct, then AFᵢ might be different in the presence of allele j vs. allele k. In some implementations, it is convenient to linearize these equations by taking the logarithm of the expression above, obtaining:

$$log ( {k}_{i} ) - log ( {k}_{j} ) = log ( {AF}_{i} ) - log ( {AF}_{j} )$$

With n alleles, there are a total of n(n−1) pairs of alleles. Thus, one may obtain a system of n(n−1) linear equations of the form above, that express relative binding propensities of alleles in terms of observed allele frequencies. If empirical allele frequency data is available for all possible pairs of alleles, this system may be solved in a straightforward manner.

However, even if empirical allele frequency data is available for only a subset of the possible pairs, a useful estimate may still be made by an error-minimization approach. The above system of equations can be expressed as the form Ax=b, where A is a matrix with n(n−1) rows and n columns, in which all entries are 0 in each row, except for a 1 term in one column and a −1 term in another, such that no two rows are equal. The vector x is a column vector having the component log kᵢ in the i-th position, and b is a column vector with each component of the form log(AFₙ)−log(AFₘ), with the values of n and m corresponding to the positions of the nonzero terms of A in the corresponding row. In some implementations, a row of the matrix can be modified so its only nonzero term is equal to 1, in some position (column m, for example). This is tantamount to arbitrarily setting the relative binding propensity of the bait molecule to allele m equal to 1, thereby setting the scale against which other relative binding propensities are measured.

If not all kᵢ and/or AFᵢ terms are known, then a practical estimate may be arrived at by defining error terms E_{i,j} by the expression

$${E}_{ij} = \frac{{k}_{i}}{{k}_{j}} - \frac{{AF}_{i}}{{AF}_{j}}$$

and selecting the unknown kᵢ and/or AFᵢ terms to minimize the total error (or some mathematical function thereof; e.g., an absolute value, the squared value, etc.). In some implementations, this minimization may be performed subject to other constraints, e.g. the requirement that the median value of all the kᵢ terms is equal to 1. In some implementations, the error is minimized by performing a least-squares optimization, although other optimization methods are suitable.

With the kᵢ terms having been either empirically determined or computed according to the previous paragraph, they can be used to re-scale the raw, measured allele frequencies from a hybrid capture process, thereby mitigating sampling bias that existed as a result of the factors described above.

Accordingly, LOH of one or more HLA-I genes may be detected by a method comprising one or more of the techniques described above for mitigating sampling bias/error that can occur in the hybrid capture process as described above. In some embodiments, the methods comprise determining allele frequency. In some embodiments, the methods comprise one or more, or all of the following steps: (a) obtaining an observed allele frequency for an allele of a gene (e.g., an HLA-I gene), wherein the observed allele frequency corresponds to frequency of nucleic acid(s) encoding at least a portion of the allele as detected among a plurality of sequence reads corresponding to the gene, wherein the plurality of sequence reads was obtained by sequencing nucleic acids encoding the gene or a portion thereof as captured by hybridization with a bait molecule; (b) obtaining a relative binding propensity for the allele to the bait molecule, wherein the relative binding propensity of the allele corresponds to propensity of nucleic acid encoding at least a portion of the allele to bind the bait molecule in the presence of nucleic acids encoding portions of one or more other alleles of the gene; (c) applying an objective function to measure a difference between the relative binding propensity and the observed allele frequency of the allele; (d) applying an optimization model to minimize the objective function; and (e) determining an adjusted allele frequency of the allele based on the optimization model and the observed allele frequency. In some embodiments, the methods further comprise determining that LOH of the gene has occurred when the adjusted allele frequency of the allele is less than a predetermined threshold.

### Optimization Modeling

Optimization refers to the method and process of working toward a solution which may be the best available solution, a preferred solution, or a solution that offers a specific benefit within a range of constraints; or continually improving; or refining; or searching for a high point or maximum (or a low point or a minimum) for an objective; or processing to reduce a penalty function or cost function; etc. In optimization modeling, the objective is often to minimize the model error, also known as the residuals of the model (a residual being the difference between an observed value and the fitted value provided by the model).

Generally, an optimization model has three main components: a) an objective function, which is the function that needs to be optimized (e.g., minimize error of parameter estimation of the model); b) a collection of variables, wherein the solution to the optimization problem is the set of values of the variables for which the objective function reaches its optimal value; and c) a collection of constraints that restrict the values of the variables. Various optimization models are known in the art and may be used in the methods of the present disclosure. One of skill in the art would be able to ascertain the suitable optimization model to use according to their specific needs and criteria. Examples of optimization models in the art include, but are not limited to, least squares regression models, logistic regression models, quadratic regression models, loess regression models, Bayesian ridge regression models, lasso regression models, elastic net regression models, decision tree models, gradient boosted tree models, neural network models, and support vector machine models. Further descriptions regarding optimization modeling can be found, e.g., in Yang, X. (2008). Introduction to mathematical optimization. From Linear Programming to Metaheuristics; Allaire, G., & Allaire, G. (2007). Numerical analysis and optimization: an introduction to mathematical modelling and numerical simulation. Oxford university press; Pedregal, P. (2006). Introduction to optimization (Vol. 46). Springer Science & Business Media; Chong, E. K., & Zak, S. H. (2004). An introduction to optimization. John Wiley & Sons; and the like.

### Allele Frequencies

In some embodiments, the optimization model comprises allele frequencies as model variables. Allele frequency is the frequency of an allele (i.e., a variant of nucleotide sequence) at a genomic locus in a population of alleles, expressed as a fraction or percentage. When the population of alleles refers to a population of alleles of one individual subject, the frequency of an allele can be calculated as the ratio of the sequence counts of the allele to the total sequence counts of all alleles at a given genomic locus of the individual subject. In this sense, the allele frequency represents the allelic composition of the individual at the genomic locus, from which the zygosity (e.g. homozygous or heterozygous) can be inferred. For instance, for a diploid individual subject, such as a human:
1) if the allele frequency of an allele has a value of, or reasonably close to (e.g., within the statistical confidence interval of), 0, then the individual subject is considered homozygous null (also known as nullizygous) for this allele;
2) if the allele frequency of an allele has a value of, or reasonably close to (e.g., within the statistical confidence interval of), 0.5, then the individual subject is considered heterozygous for this allele; and
3) if the allele frequency of an allele has a value of, or reasonably close to (e.g., within the statistical confidence interval of), 1, then the individual subject is considered homozygous for this allele.

In some embodiments, the allele frequency is an observed allele frequency, corresponding to relative frequency of nucleic acid(s) encoding at least a portion of the allele as detected among the plurality of sequence reads, as compared to a reference value. In some embodiments, the reference value is a total number of sequence reads. In some embodiments, the reference value is a number of sequence reads corresponding to a reference gene, or a function thereof, such as reads per million mapped reads (RPM) or counts per million mapped reads (CPM).

In some embodiments, the allele frequency can be expressed as the relative binding propensity. In a hybrid capture-based sequencing process, the relative binding propensity corresponds to the likelihood of one allele binding to the bait molecule in the presence of one or more other alleles. Accordingly, in some embodiments, an optimization model is applied to an objective function that measures a difference between the relative binding propensity of one allele and the observed allele frequency of the allele.

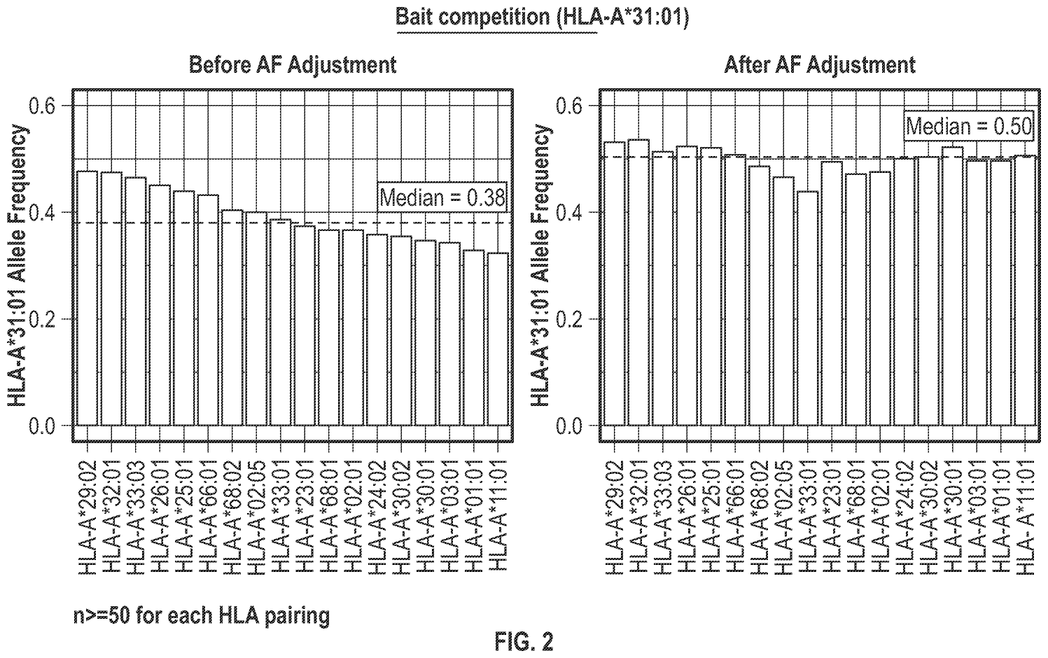

By way of example, FIG. 2 illustrates the result of such a scaling for various HLA-A alleles. The bar chart on the left indicates raw allele frequencies from heterozygous subjects of the HLA-A*31:01 allele in the presence of various other HLA-A alleles indicated on the horizontal axis. The median allele frequency is 0.38, indicating that HLA-A*31:01 is typically under-sampled in the presence of the indicated other alleles. After correcting the bias, the chart on the right indicates a median allele frequency of 0.5, which is more consistent with a heterozygous sample population.

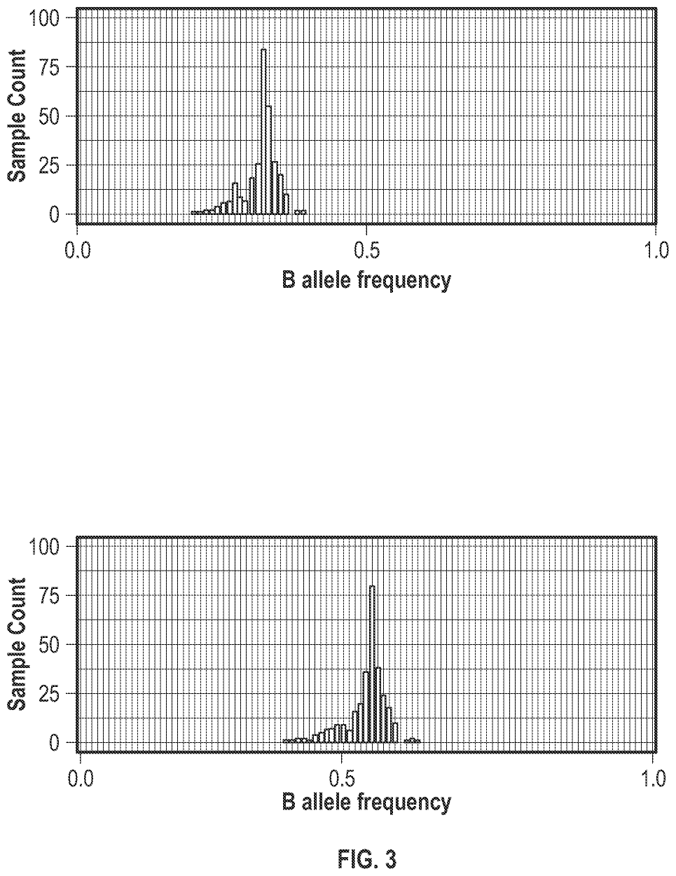

FIG. 3 illustrates the effect of adjusted allele frequencies for use in determining loss-of-heterozygosity (LOH) for the human leukocyte antigen class I (HLA-I) gene in a population of individuals. The X-axis shows the B allele frequency (BAF) for each individual in the population, wherein the B allele refers to the non-reference allele, or the minor allele. The Y-axis shows the sample count of the BAF in the population. FIG. 3 shows that after adjusting the allele frequencies using the methods described herein, the median allele frequency is adjusted from around 0.32 (upper panel) to around 0.5 (lower panel), suggesting most of the population of individuals are heterozygous for the HLA-I gene.

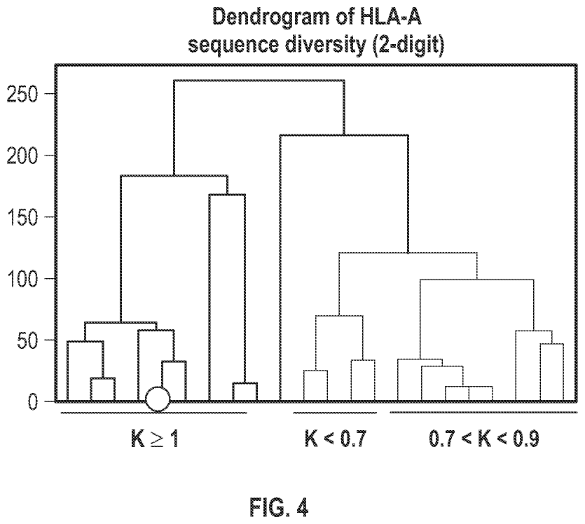

As shown in FIG. 4, particular alleles (or fragments thereof) may have a range of relative binding propensities to a particular bait molecule. In order to improve capture of sequences representing the full polymorphic variation of the gene, one may wish to select one or more additional bait molecule(s), particularly those that have improved binding propensities to alleles with a lower relative binding propensity to the original bait molecule.

As such, in some embodiments, the methods of the present disclosure may include obtaining an observed allele frequency for two or more alleles of a gene (e.g., an HLA-I gene); obtaining a relative binding propensity for two or more alleles of a gene (e.g., an HLA-I gene) to a specific bait molecule; and/or identifying or selecting the sequence of a second bait molecule. In some embodiments, one or more alleles of the gene with a lower relative binding propensity to a first bait molecule may have a higher binding propensity to the second bait molecule than to the first bait molecule. For example, the second bait molecule can comprise a sequence complementary to at least a portion of one of the lower-binding alleles of the gene, or to a sequence (e.g., a consensus sequence) based on complementarity or binding to the sequence(s) of one or more lower-binding alleles of the gene. This allows for bait selection based on the sequences of lower-binding alleles of a polymorphic gene, e.g., in order to sample the diversity of the gene more comprehensively or with less bias (e.g., based on hybrid capture).

### Least Squares Optimization

In some embodiments, the optimization model is a least squares optimization model. A least squares optimization model is a regression optimization model wherein the objective function is a quadratic function (e.g., a sum of squares function) of the parameters to be optimized (e.g., variable residuals/error to be minimized). In some embodiments, a least squares optimization model is used in the methods described herein to minimize an objective function which measures a difference between the relative binding propensity and the observed allele frequency of an allele. In some embodiments, the optimization model is a quadratic regression. In some embodiments, the optimization model is a loess regression.

In some embodiments, the optimization model may be used to correct or adjust variables of interest (e.g., allele frequencies). In some embodiments, an optimization model and the observed allele frequency of an allele are used to determine the adjusted allele frequency of the allele. The adjusted allele frequency can further be used in downstream operations, e.g., inferring the zygosity status of the individual subject for the allele.

Further descriptions of least squares optimization can be found, e.g., in Wolberg, J. (2006). Data analysis using the method of least squares: extracting the most information from experiments. Springer Science & Business Media; Borowiak, D. (2001). Linear models, least squares and alternatives; Björck, Å. (1996). Numerical methods for least squares problems. Society for Industrial and Applied Mathematics; Luenberger, D. G. (1997) “Least-Squares Estimation”. Optimization by Vector Space Methods. New York: John Wiley & Sons. pp. 78-102; and the like.

### Model Constraints

In some embodiments, the optimization model is subject to one or more constraints. Constraints limit the possible values for the variables in an optimization model. In some embodiments, the one or more constraints require that median value of the relative binding propensities for a plurality of alleles of the gene is equal to 0. In some embodiments, the one or more constraints require that median value of the relative binding propensities for a plurality of alleles of the gene is equal to 0.5. In some embodiments, the one or more constraints require that median value of the relative binding propensities for a plurality of alleles of the gene is equal to 1.

### Sequencing

In some embodiments, the plurality of sequence reads was obtained by performing sequencing on nucleic acids captured by hybridization with the bait molecule. In some embodiments, the plurality of sequence reads was obtained by performing whole exome sequencing on nucleic acids captured by hybridization with the bait molecule. In some embodiments, the plurality of sequence reads was obtained by performing next-generation sequencing (NGS), whole exome sequencing, or methylation sequencing on nucleic acids captured by hybridization with the bait molecule.

In some embodiments, the methods further comprise, prior to obtaining the observed allele frequency: sequencing a plurality of polynucleotides by next-generation sequencing (NGS) in order to obtain the plurality of sequence reads, wherein the plurality of polynucleotides comprises nucleic acid(s) encoding at least a portion of the allele. NGS methods are known in the art, and are described, e.g., in Metzker, M. (2010) Nature Biotechnology Reviews 11:31-46. Platforms for next-generation sequencing include, e.g., Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, and Pacific Biosciences' PacBio RS system. NGS technologies can include one or more of steps, e.g., template preparation, sequencing and imaging, and data analysis. Methods for template preparation can include steps such as randomly breaking nucleic acids (e.g., genomic DNA) into smaller sizes and generating sequencing templates (e.g., fragment templates or mate-pair templates). The spatially separated templates can be attached or immobilized to a solid surface or support, allowing massive amounts of sequencing reactions to be performed simultaneously. Types of templates that can be used for NGS reactions include, e.g., clonally amplified templates originating from single DNA molecules, and single DNA molecule templates. Exemplary sequencing and imaging steps for NGS include, e.g., cyclic reversible termination (CRT), sequencing by ligation (SBL), single-molecule addition (pyrosequencing), and real-time sequencing. After NGS reads have been generated, they can be aligned to a known reference sequence or assembled de novo. For example, identifying genetic variations such as single-nucleotide polymorphism and structural variants in a sample (e.g., a tumor sample) can be accomplished by aligning NGS reads to a reference sequence (e.g., a wild type sequence). Methods of sequence alignment for NGS are described e.g., in Trapnell C. and Salzberg S. L. Nature Biotech., 2009, 27:455-457. Examples of de novo assemblies are described, e.g., in Warren R. et al., Bioinformatics, 2007, 23:500-501; Butler J. et al., Genome Res., 2008, 18:810-820; and Zerbino D. R. and Birney E., Genome Res., 2008, 18:821-829. Sequence alignment or assembly can be performed using read data from one or more NGS platforms, e.g., mixing Roche/454 and Illumina/Solexa read data. In some embodiments, NGS is performed according to the methods described in, e.g., Frampton, G. M. et al. (2013) Nat. Biotech. 31:1023-1031; and/or Montesion, M., et al., Cancer Discovery (2021) 11(2):282-92.

In some embodiments, the methods further comprise, prior to obtaining the observed allele frequency: sequencing a plurality of polynucleotides by whole exome sequencing in order to obtain the plurality of sequence reads, wherein the plurality of polynucleotides comprises nucleic acid(s) encoding at least a portion of the allele.

In some embodiments, the methods further comprise, prior to sequencing the plurality of polynucleotides: contacting a mixture of polynucleotides with the bait molecule under conditions suitable for hybridization, wherein the mixture comprises a plurality of polynucleotides capable of hybridization with the bait molecule; and isolating a plurality of polynucleotides that hybridized with the bait molecule, wherein the isolated plurality of polynucleotides that hybridized with the bait molecule are sequenced by NGS. FIG. 1 illustrates such a hybrid capture process. Further details about this and other hybrid capture processes can be found in U.S. Pat. No. 9,340,830; Frampton, G. M. et al. (2013) Nat. Biotech. 31:1023-1031; and Montesion, M., et al., Cancer Discovery (2021) 11(2):282-92, the entirety of each which is incorporated by reference herein. In some embodiments, the methods further comprise, prior to contacting the mixture of polynucleotides with the bait molecule: obtaining a sample from an individual, wherein the sample comprises tumor cells and/or tumor nucleic acids; and extracting the mixture of polynucleotides from the sample, wherein the mixture of polynucleotides is from the tumor cells and/or tumor nucleic acids. In some embodiments, the sample further comprises non-tumor cells.

In some embodiments, the methods comprise subjecting a plurality of polynucleotides to methylation sequencing in order to obtain the plurality of sequence reads. In some embodiments, the plurality of polynucleotides comprises nucleic acid(s) encoding at least a portion of the allele.

In some embodiments, nucleic acids are obtained from a sample, e.g., comprising tumor cells and/or tumor nucleic acids. For example, the sample can comprise tumor cell(s), circulating tumor cell(s), tumor nucleic acids (e.g., tumor circulating tumor DNA, cfDNA, or cfRNA), part or all of a tumor biopsy, fluid, cells, tissue, mRNA, DNA, RNA, cell-free DNA, and/or cell-free RNA. In some embodiments, the sample is from a tumor biopsy or tumor specimen. In some embodiments, the sample further comprises non-tumor cells and/or non-tumor nucleic acids. In some embodiments, the fluid comprises blood, serum, plasma, saliva, semen, cerebral spinal fluid, amniotic fluid, peritoneal fluid, interstitial fluid, etc.

In some embodiments, the sample is or comprises biological tissue or fluid. The sample can contain compounds that are not naturally intermixed with the tissue in nature such as preservatives, anticoagulants, buffers, fixatives, nutrients, antibiotics or the like. In one embodiment, the sample is preserved as a frozen sample or as a formaldehyde- or paraformaldehyde-fixed paraffin-embedded (FFPE) tissue preparation. For example, the sample can be embedded in a matrix, e.g., an FFPE block or a frozen sample. In another embodiment, the sample is a blood or blood constituent sample. In yet another embodiment, the sample is a bone marrow aspirate sample. In another embodiment, the sample comprises cell-free DNA (cfDNA). Without wishing to be bound by theory, it is believed that in some embodiments, cfDNA is DNA from apoptosed or necrotic cells. Typically, cfDNA is bound by protein (e.g., histone) and protected by nucleases. CfDNA can be used as a biomarker, for example, for non-invasive prenatal testing (NIPT), organ transplant, cardiomyopathy, microbiome, and cancer. In another embodiment, the sample comprises circulating tumor DNA (ctDNA). Without wishing to be bound by theory, it is believed that in some embodiments, ctDNA is cfDNA with a genetic or epigenetic alteration (e.g., a somatic alteration or a methylation signature) that can discriminate it originating from a tumor cell versus a non-tumor cell. In another embodiment, the sample comprises circulating tumor cells (CTCs). Without wishing to be bound by theory, it is believed that in some embodiments, CTCs are cells shed from a primary or metastatic tumor into the circulation. In some embodiments, CTCs apoptose and are a source of ctDNA in the blood/lymph.

In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as ductal lavages or bronchoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained.

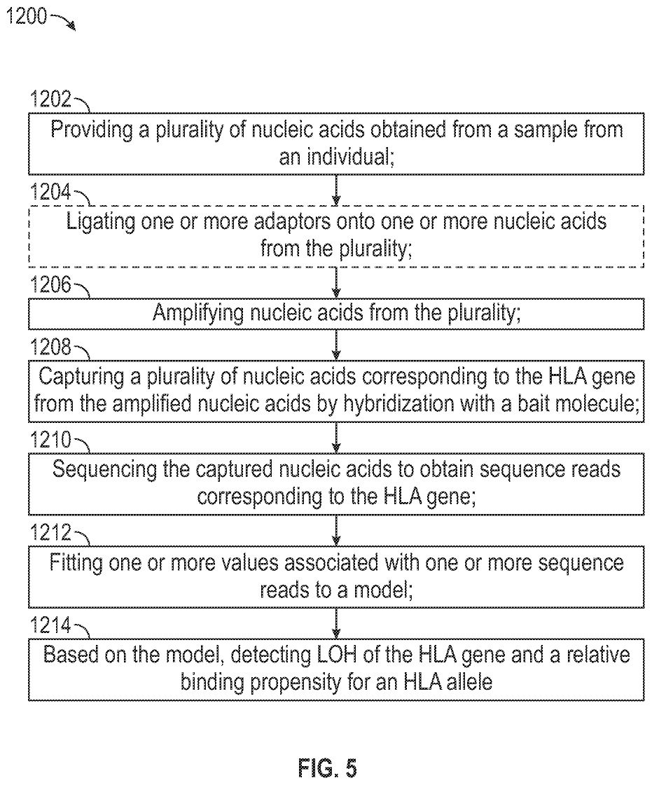

FIG. 5 illustrates an exemplary process 1200 for detecting loss-of-heterozygosity (LOH) of a human leukocyte antigen (HLA) gene, e.g., an HLA-I gene, in accordance with some embodiments. Process 1200 is performed, for example, using one or more electronic devices implementing a software program. In some examples, process 1200 is performed using a client-server system, and the blocks of process 1200 are divided up in any manner between the server and a client device. In other examples, the blocks of process 1200 are divided up between the server and multiple client devices. Thus, while portions of process 1200 are described herein as being performed by particular devices of a client-server system, it will be appreciated that process 1200 is not so limited. In other examples, process 1200 is performed using only a client device or only multiple client devices. In process 1200, some blocks are, optionally, combined, the order of some blocks is, optionally, changed, and some blocks are, optionally, omitted. In some examples, additional steps may be performed in combination with the process 1200. Accordingly, the operations as illustrated (and described in greater detail below) are exemplary by nature and, as such, should not be viewed as limiting.

At block 1202, a plurality of nucleic acids obtained from a sample from an individual are provided (e.g., from an individual having a squamous cell cancer or NSCLC), wherein the plurality of nucleic acids comprises nucleic acids encoding an HLA gene (e.g., an HLA-I gene). Optionally, at block 1204, one or more adaptors are ligated onto one or more nucleic acids from the plurality of nucleic acids. At block 1206, nucleic acids are amplified from the plurality of nucleic acids. At block 1208, a plurality of nucleic acids corresponding to the HLA gene (e.g., an HLA-I gene) are captured from the amplified nucleic acids by hybridization with a bait molecule. At block 1210, an exemplary sequencer sequences the captured nucleic acids to obtain a plurality of sequence reads corresponding to the HLA gene (e.g., an HLA-I gene). At block 1212, an exemplary system (e.g., one or more electronic devices) fits one or more values associated with one or more of the plurality of sequence reads to a model. At block 1214, the system detects LOH of the HLA gene (e.g., an HLA-I gene) and a relative binding propensity for an HLA allele of the HLA gene (e.g., an HLA-I gene) based on the model.

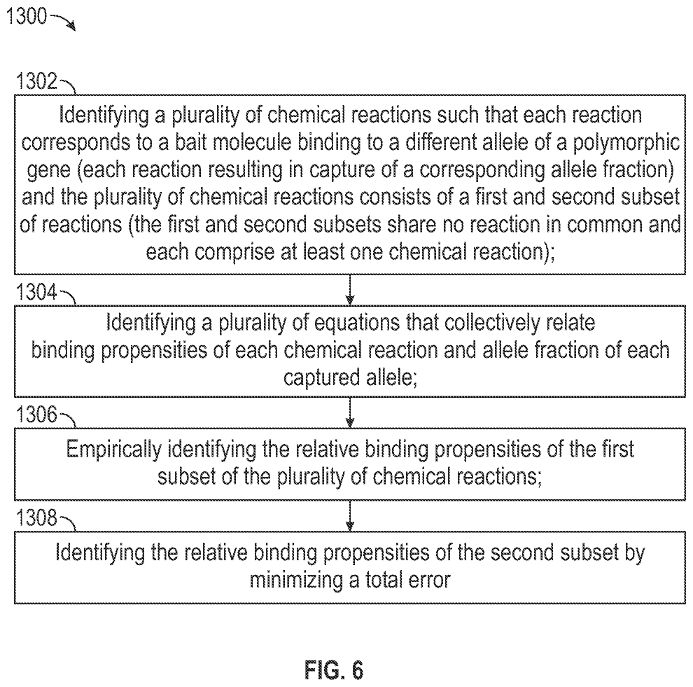

FIG. 6 illustrates an exemplary process 1300 for identifying relative binding propensities of different alleles of a polymorphic gene (e.g., an HLA-I gene) to a bait molecule, in accordance with some embodiments. Process 1300 is performed, for example, using one or more electronic devices implementing a software program. In some examples, process 1300 is performed using a client-server system, and the blocks of process 1300 are divided up in any manner between the server and a client device. In other examples, the blocks of process 1300 are divided up between the server and multiple client devices. Thus, while portions of process 1300 are described herein as being performed by particular devices of a client-server system, it will be appreciated that process 1300 is not so limited. In other examples, process 1300 is performed using only a client device or only multiple client devices. In process 1300, some blocks are, optionally, combined, the order of some blocks is, optionally, changed, and some blocks are, optionally, omitted. In some examples, additional steps may be performed in combination with the process 1300. Accordingly, the operations as illustrated (and described in greater detail below) are exemplary by nature and, as such, should not be viewed as limiting.

At block 1302, an exemplary system (e.g., one or more electronic devices) identifies a plurality of chemical reactions, e.g., such that each reaction corresponds to a bait molecule binding to a different allele of a polymorphic gene (e.g., an HLA-I gene), and each reaction resulting in capture of a corresponding allele fraction, and the plurality of chemical reactions consists of a first subset of reactions and a second subset of reactions, in which the first and second subsets share no reaction in common and in which the first and second subsets each comprise at least one chemical reaction. At block 1304, the system identifies a plurality of equations that collectively relate binding propensities of each chemical reaction and allele fraction of each captured allele. At block 1306, the system empirically identifies the relative binding propensities of the first subset of the plurality of chemical reactions. At block 1308, the system identifies the relative binding propensities of the second subset by minimizing a total error.

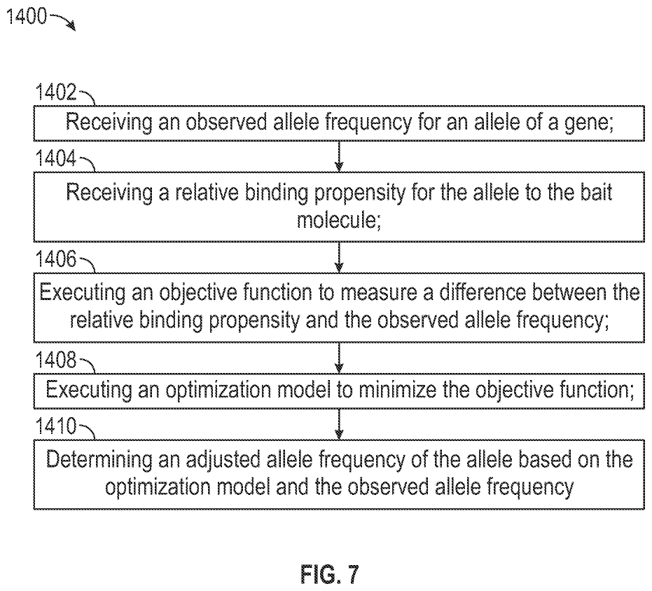

FIG. 7 illustrates an exemplary process 1400 for determining allele frequency, in accordance with some embodiments. In some embodiments, the allele frequency of one or more HLA alleles (e.g., of one or more HLA-I genes) is determined, e.g., to detect LOH. Process 1400 is performed, for example, using one or more electronic devices implementing a software program. In some examples, process 1400 is performed using a client-server system, and the blocks of process 1400 are divided up in any manner between the server and a client device. In other examples, the blocks of process 1400 are divided up between the server and multiple client devices. Thus, while portions of process 1400 are described herein as being performed by particular devices of a client-server system, it will be appreciated that process 1400 is not so limited. In other examples, process 1400 is performed using only a client device or only multiple client devices. In process 1400, some blocks are, optionally, combined, the order of some blocks is, optionally, changed, and some blocks are, optionally, omitted. In some examples, additional steps may be performed in combination with the process 1400. Accordingly, the operations as illustrated (and described in greater detail below) are exemplary by nature and, as such, should not be viewed as limiting.

At block 1402, an exemplary system (e.g., one or more electronic devices) receives an observed allele frequency for an allele of a gene (e.g., an HLA-I gene). In some embodiments, the observed allele frequency corresponds to frequency of nucleic acid(s) encoding at least a portion of the allele as detected among a plurality of sequence reads corresponding to the gene, and the plurality of sequence reads was obtained by sequencing nucleic acids encoding the gene or a portion thereof as captured by hybridization with a bait molecule. In some embodiments, the gene is a human HLA gene (e.g., a human HLA-I gene), and the alleles are human HLA alleles (e.g., human alleles of a human HLA-I gene, e.g., as known in the art and/or described herein). At block 1404, the system receives a relative binding propensity for the allele to the bait molecule. In some embodiments, the relative binding propensity of the allele corresponds to propensity of nucleic acid encoding at least a portion of the allele to bind the bait molecule in the presence of nucleic acids encoding portions of one or more other alleles of the gene. At block 1406, the system executes an objective function to measure a difference between the relative binding propensity and the observed allele frequency of the allele. At block 1408, the system executes an optimization model to minimize the objective function. At block 1410, the system determines an adjusted allele frequency of the allele based on the optimization model and the observed allele frequency.

In some embodiments according to any of the embodiments described herein, the gene is a human leukocyte antigen (HLA) gene encoding a major histocompatibility (MHC) class I molecule. In some embodiments, the methods further comprise, after determining the adjusted allele frequency: determining that the gene has undergone loss-of-heterozygosity (LOH) based at least in part on the adjusted allele frequency.

In some embodiments, provided herein are methods for detecting loss-of-heterozygosity (LOH) of a human leukocyte antigen (HLA) gene, e.g., an HLA class I gene. In some embodiments, the methods comprise: a) obtaining an observed allele frequency for an HLA allele, wherein observed allele frequency corresponds to frequency of nucleic acid(s) encoding at least a portion of the HLA allele as detected among a plurality of sequence reads corresponding to an HLA gene (e.g., an HLA-I gene), wherein the plurality of sequence reads was obtained by sequencing nucleic acids encoding the gene or a portion thereof as captured by hybridization with a bait molecule; b) obtaining a relative binding propensity for the HLA allele to the bait molecule, wherein the relative binding propensity of the HLA allele corresponds to propensity of nucleic acid encoding at least a portion of the HLA allele to bind the bait molecule in the presence of nucleic acids encoding portions of one or more other HLA alleles; c) applying an objective function to measure a difference between the relative binding propensity and the observed allele frequency of the HLA allele; d) applying an optimization model to minimize the objective function; e) determining an adjusted allele frequency of the HLA allele based on the optimization model and the observed allele frequency; and f) determining that LOH has occurred when the adjusted allele frequency of the HLA allele is less than a predetermined threshold. In some embodiments, the HLA gene is a human HLA-A, HLA-B, or HLA-C gene.

In some embodiments, the plurality of sequence reads was obtained by sequencing nucleic acids obtained from a sample comprising tumor cells and/or tumor nucleic acids. In some embodiments, the sample further comprises non-tumor cells. Accordingly, in some embodiments, the methods comprise one or more, or all, of the following steps: (a) providing a plurality of nucleic acids obtained from a sample from an individual (e.g., an individual having squamous cell cancer or NSCLC), wherein the plurality of nucleic acids comprises nucleic acids encoding an HLA-I gene; (b) optionally, ligating one or more adaptors onto one or more nucleic acids from the plurality of nucleic acids; (c) amplifying nucleic acids from the plurality of nucleic acids; (d) capturing a plurality of nucleic acids corresponding to the HLA-I gene, wherein the plurality of nucleic acids corresponding to the HLA-I gene is captured from the amplified nucleic acids by hybridization with a bait molecule; (e) sequencing, by a sequencer, the captured nucleic acids to obtain a plurality of sequence reads corresponding to the HLA-I gene; (f) fitting, by one or more processors, one or more values associated with one or more of the plurality of sequence reads to a model; and (g) based on the model, detecting the somatic LOH of one or more HLA-I genes and a relative binding propensity for an HLA allele of the HLA-I gene. In some embodiments, the somatic LOH of one or more HLA-I genes and relative binding propensity for an HLA allele of the HLA-I gene are detected by one or more, or all, of the following steps: (a) obtaining an observed allele frequency for an HLA allele of an HLA-I gene, wherein the observed allele frequency corresponds to the frequency of nucleic acid(s) encoding at least a portion of the HLA allele as detected among the plurality of sequence reads corresponding to the HLA-I gene; (b) obtaining a relative binding propensity for the HLA allele to the bait molecule, wherein the relative binding propensity of the HLA allele corresponds to propensity of a nucleic acid encoding at least a portion of the HLA allele to bind the bait molecule in the presence of nucleic acids encoding portions of one or more other HLA alleles; (c) applying an objective function to measure a difference between the relative binding propensity and the observed allele frequency of the HLA allele; (d) applying an optimization model to minimize the objective function; (e) determining an adjusted allele frequency of the HLA allele based on the optimization model and the observed allele frequency; and (f) determining that LOH of one or more HLA-I genes has occurred when the adjusted allele frequency of the HLA allele is less than a predetermined threshold.

Another exemplary approach that may be used to detect LOH of one or more HLA-I genes includes the methods described by Pyke et al., Sensitive HLA loss of heterozygosity detection reveals allele-specific neoantigen expansion as resistance mechanism to anti-PD-1 therapy [abstract]. In: Proceedings of the Annual Meeting of the American Association for Cancer Research 2020; 2020 Apr. 27-28 and Jun. 22-24. Philadelphia (PA): AACR; Cancer Res 2020; 80(16 Suppl):Abstract nr 6678, e.g., available at the website: www[dot]personalis[dot]com/wp-content/uploads/bsk-pdf-manager/2020/07/2020_AACR-Poster-DASH_PostedVersion.pdf.

Another exemplary approach that may be used to detect LOH of one or more HLA-I genes includes a method comprising sequencing a tumor sample from an individual and a non-tumor sample from the individual (e.g., a normal sample); genotyping the samples to determine the HLA-I gene alleles present in each sample; comparing the HLA-I gene alleles present in each sample; and determining that LOH of an HLA-I gene has occurred if the tumor sample exhibits homozygosity of an HLA-I allele and the non-tumor sample exhibits heterozygosity of the corresponding HLA-I allele.

### Software and Devices

In some other aspects, provided herein are non-transitory computer-readable storage media. In some embodiments, the non-transitory computer-readable storage media comprise one or more programs for execution by one or more processors of a device, the one or more programs including instructions which, when executed by the one or more processors, cause the device to perform the method according to any of the embodiments described herein.

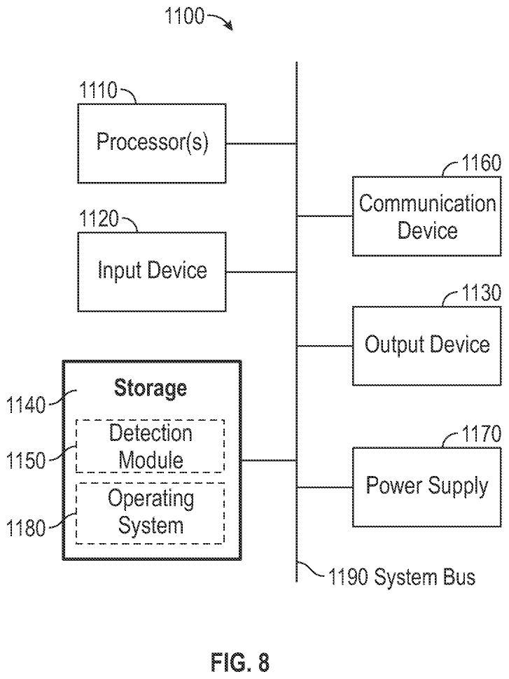

FIG. 8 illustrates an example of a computing device in accordance with one embodiment. Device 1100 can be a host computer connected to a network. Device 1100 can be a client computer or a server. As shown in FIG. 8, device 1100 can be any suitable type of microprocessor-based device, such as a personal computer, workstation, server or handheld computing device (portable electronic device) such as a phone or tablet. The device can include, for example, one or more of processor(s) 1110, input device 1120, output device 1130, storage 1140, communication device 1160, power supply 1170, operating system 1180, and system bus 1190. Input device 1120 and output device 1130 can generally correspond to those described herein, and can either be connectable or integrated with the computer.

Input device 1120 can be any suitable device that provides input, such as a touch screen, keyboard or keypad, mouse, or voice-recognition device. Output device 1130 can be any suitable device that provides output, such as a touch screen, haptics device, or speaker.

Storage 1140 can be any suitable device that provides storage (e.g., an electrical, magnetic or optical memory including a RAM (volatile and non-volatile), cache, hard drive, or removable storage disk). Communication device 1160 can include any suitable device capable of transmitting and receiving signals over a network, such as a network interface chip or device. The components of the computer can be connected in any suitable manner, such as via a wired media (e.g., a physical bus, ethernet, or any other wire transfer technology) or wirelessly (e.g., Bluetooth®, Wi-Fi®, or any other wireless technology). For example, in FIG. 8, the components are connected by System Bus 1190.

Detection module 1150, which can be stored as executable instructions in storage 1140 and executed by processor(s) 1110, can include, for example, the processes that embody the functionality of the present disclosure (e.g., as embodied in the devices as described herein).

Detection module 1150 can also be stored and/or transported within any non-transitory computer-readable storage medium for use by or in connection with an instruction execution system, apparatus, or device, such as those described herein, that can fetch instructions associated with the software from the instruction execution system, apparatus, or device and execute the instructions. In the context of this disclosure, a computer-readable storage medium can be any medium, such as storage 1140, that can contain or store processes for use by or in connection with an instruction execution system, apparatus, or device. Examples of computer-readable storage media may include memory units like hard drives, flash drives and distribute modules that operate as a single functional unit. Also, various processes described herein may be embodied as modules configured to operate in accordance with the embodiments and techniques described above. Further, while processes may be shown and/or described separately, those skilled in the art will appreciate that the above processes may be routines or modules within other processes.

Detection module 1150 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

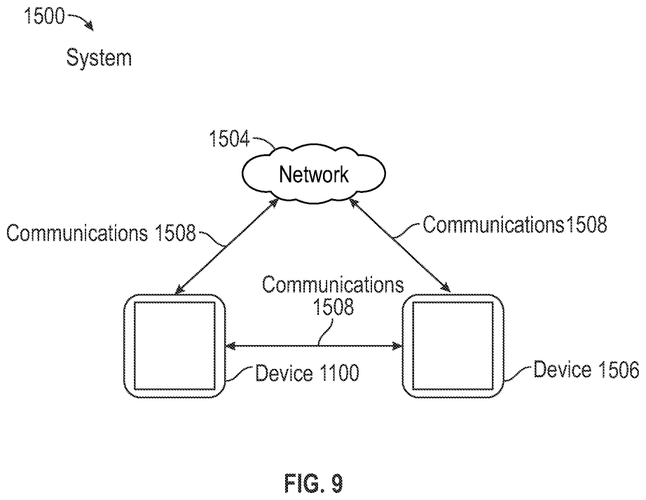

Device 1100 may be connected to a network (e.g., Network 1504, as shown in FIG. 9 and/or described below), which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1100 can implement any operating system (e.g., Operating System 1180) suitable for operating on the network. Detection module 1150 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example. In some embodiments, Operating System 1180 is executed by one or more processors, e.g., Processor(s) 1110.

Device 1100 can further include Power Supply 1170, which can be any suitable power supply.

In some embodiments, Detection module 1150 is a module for detecting LOH of one or more HLA-I genes and/or tumor mutational burden and includes the processes that embody the functionality of the present disclosure (e.g., as embodied in the devices as described herein).

FIG. 9 illustrates an example of a computing system in accordance with one embodiment. In System 1500, Device 1100 (e.g., as described above and illustrated in FIG. 8) is connected to Network 1504, which is also connected to Device 1506. In some embodiments, Device 1506 is a sequencer. Exemplary sequencers can include, without limitation, Roche/454's Genome Sequencer (GS) FLX System, Illumina/Solexa's Genome Analyzer (GA), Illumina's HiSeq 2500, HiSeq 3000, HiSeq 4000 and NovaSeq 6000 Sequencing Systems, Life/APG's Support Oligonucleotide Ligation Detection (SOLiD) system, Polonator's G.007 system, Helicos BioSciences' HeliScope Gene Sequencing system, or Pacific Biosciences' PacBio RS system. Devices 1100 and 1506 may communicate, e.g., using suitable communication interfaces via Network 1504, such as a Local Area Network (LAN), Virtual Private Network (VPN), or the Internet. In some embodiments, Network 1504 can be, for example, the Internet, an intranet, a virtual private network, a cloud network, a wired network, or a wireless network. Devices 1100 and 1506 may communicate, in part or in whole, via wireless or hardwired communications, such as Ethernet, IEEE 802.11b wireless, or the like. Additionally, Devices 1100 and 1506 may communicate, e.g., using suitable communication interfaces, via a second network, such as a mobile/cellular network. Communication between Devices 1100 and 1506 may further include or communicate with various servers such as a mail server, mobile server, media server, telephone server, and the like. In some embodiments, Devices 1100 and 1506 can communicate directly (instead of, or in addition to, communicating via Network 1504), e.g., via wireless or hardwired communications, such as Ethernet, IEEE 802.11b wireless, or the like. In some embodiments, Devices 1100 and 1506 communicate via Communications 1508, which can be a direct connection or can occur via a network (e.g., Network 1504).

One or all of Devices 1100 and 1506 generally include logic (e.g., http web server logic) or is programmed to format data, accessed from local or remote databases or other sources of data and content, for providing and/or receiving information via Network 1504 according to various examples described herein.

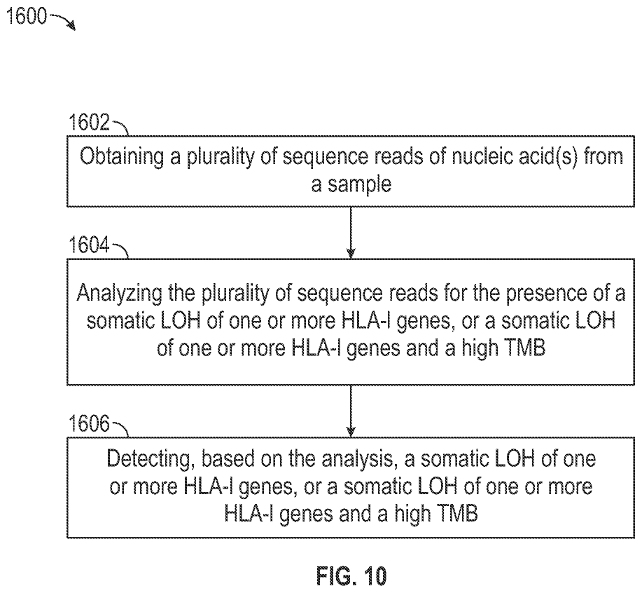

FIG. 10 illustrates an exemplary process 1600 for detecting a somatic LOH of one or more HLA-I genes, or a somatic LOH of one or more HLA-I genes and a high TMB, in accordance with some embodiments of the present disclosure. Process 1600 is performed, for example, using one or more electronic devices implementing a software program. In some examples, process 1600 is performed using a client-server system, and the blocks of process 1600 are divided up in any manner between the server and a client device. In other examples, the blocks of process 1600 are divided up between the server and multiple client devices. Thus, while portions of process 1600 are described herein as being performed by particular devices of a client-server system, it will be appreciated that process 1600 is not so limited. In some embodiments, the executed steps can be executed across many systems, e.g., in a cloud environment. In other examples, process 1600 is performed using only a client device or only multiple client devices. In process 1600, some blocks are, optionally, combined, the order of some blocks is, optionally, changed, and some blocks are, optionally, omitted. In some examples, additional steps may be performed in combination with the process 1600. Accordingly, the operations as illustrated (and described in greater detail below) are exemplary by nature and, as such, should not be viewed as limiting.

At block 1602, a plurality of sequence reads of one or more nucleic acids is obtained, wherein the one or more nucleic acids are derived from a sample obtained from an individual. In some embodiments, the sample is obtained from an individual having a cancer, such as a squamous cell cancer or NSCLC. In some embodiments, the sequence reads are obtained using a sequencer, e.g., as described herein or otherwise known in the art. In some embodiments, the nucleic acid(s) comprise one or more nucleic acids corresponding to an HLA-I gene, or portion thereof. Optionally, prior to obtaining the sequence reads, the sample is purified, enriched (e.g., for nucleic acid(s) corresponding to an HLA-I gene, or portion thereof), and/or subjected to PCR amplification. At block 1604, an exemplary system (e.g., one or more electronic devices) analyzes the plurality of sequence reads for the presence of a somatic LOH of one or more HLA-I genes, or a somatic LOH of one or more HLA-I genes and a high TMB. At block 1606, the system detects (e.g., based on the analysis) a somatic LOH of one or more HLA-I genes, or a somatic LOH of one or more HLA-I genes and a high TMB, in the sample.

In some embodiments, the analyzing comprises one or more, or all, of the following steps: (a) determining an observed allele frequency for an HLA allele of an HLA-I gene, wherein the observed allele frequency corresponds to the frequency of nucleic acid(s) encoding at least a portion of the HLA allele as detected among a plurality of sequence reads corresponding to an HLA-I gene, wherein the plurality of sequence reads was obtained by sequencing nucleic acids encoding the HLA-I gene or a portion thereof as captured by hybridization with a bait molecule; (b) determining a relative binding propensity for the HLA allele to the bait molecule, wherein the relative binding propensity of the HLA allele corresponds to propensity of a nucleic acid encoding at least a portion of the HLA allele to bind the bait molecule in the presence of nucleic acids encoding portions of one or more other HLA alleles; (c) executing an objective function to measure a difference between the relative binding propensity and the observed allele frequency of the HLA allele; (d) executing an optimization model to minimize the objective function; (e) determining an adjusted allele frequency of the HLA allele based on the optimization model and the observed allele frequency; and (f) determining the presence of a somatic LOH of one or more HLA-I genes when the adjusted allele frequency of the HLA allele is less than a predetermined threshold.

In some embodiments, the analyzing comprises one or more, or all, of the following steps: (a) receiving, using the one or more processors, an observed allele frequency for an HLA allele of an HLA-I gene, wherein the observed allele frequency corresponds to the frequency of nucleic acid(s) encoding at least a portion of the HLA allele as detected among a plurality of sequence reads corresponding to an HLA-I gene, wherein the plurality of sequence reads was obtained by sequencing nucleic acids encoding the HLA-I gene or a portion thereof as captured by hybridization with a bait molecule; (b) receiving, using the one or more processors, a relative binding propensity for the HLA allele to the bait molecule, wherein the relative binding propensity of the HLA allele corresponds to propensity of a nucleic acid encoding at least a portion of the HLA allele to bind the bait molecule in the presence of nucleic acids encoding portions of one or more other HLA alleles; (c) executing, using the one or more processors, an objective function to measure a difference between the relative binding propensity and the observed allele frequency of the HLA allele; (d) executing, using the one or more processors, an optimization model to minimize the objective function; (e) determining, using the one or more processors, an adjusted allele frequency of the HLA allele based on the optimization model and the observed allele frequency; and (f) determining, using the one or more processors, that a somatic LOH of one or more HLA-I genes has occurred when the adjusted allele frequency of the HLA allele is less than a predetermined threshold.

In some embodiments, the analyzing comprises determining tumor mutational burden from a plurality of sequence reads, e.g., a plurality of sequence reads obtained by sequencing nucleic acids corresponding to at least a portion of a genome (such as from an enriched or unenriched sample). In some embodiments, tumor mutational burden is determined according to any of the methods described herein. In some embodiments, tumor mutational burden is determined based on the number of coding mutations per megabase of genome sequenced. In some embodiments, tumor mutational burden is determined based on the number of non-driver somatic coding mutations per megabase of genome sequenced.

In some embodiments of any of the methods, systems, devices, non-transitory computer readable storage media, or processes of the disclosure, the one or more HLA-I genes comprise one or more of a human HLA-A, HLA-B, or HLA-C gene. In some embodiments of any of the methods, systems, devices, non-transitory computer readable storage media, or processes of the disclosure, the plurality of sequence reads is obtained by sequencing nucleic acids obtained from a sample comprising squamous cell cancer or NSCLC cells and/or squamous cell cancer or NSCLC nucleic acids. In some embodiments of any of the methods, systems, devices, non-transitory computer readable storage media, or processes of the disclosure, the plurality of sequence reads is obtained by whole exome sequencing, whole genome sequencing, gene-targeted sequencing, or next-generation sequencing. In some embodiments of any of the methods, systems, devices, non-transitory computer readable storage media, or processes of the disclosure, the sample further comprises non-squamous cell cancer or non-NSCLC cells and/or non-squamous cell cancer or non-NSCLC nucleic acids. In some embodiments of any of the methods, systems, devices, non-transitory computer readable storage media, or processes of the disclosure, the sample is from a tumor biopsy, tumor specimen, or a circulating tumor cell. In some embodiments of any of the methods, systems, devices, non-transitory computer readable storage media, or processes of the disclosure, the sample comprises cell-free DNA (cfDNA) or circulating tumor DNA (ctDNA) from the squamous cell cancer or NSCLC. In some embodiments of any of the methods, systems, devices, non-transitory computer readable storage media, or processes of the disclosure, the sample comprises fluid, cells, or tissue. In some embodiments of any of the methods, systems, devices, non-transitory computer readable storage media, or processes of the disclosure, the sample comprises blood or plasma. In some embodiments of any of the methods, systems, devices, non-transitory computer readable storage media, or processes of the disclosure, the sample is a nucleic acid sample. In some embodiments of any of the methods, systems, devices, non-transitory computer readable storage media, or processes of the disclosure, the nucleic acid sample comprises mRNA, DNA, circulating tumor DNA, cell-free DNA, or cell-free RNA. In some embodiments of any of the methods, systems, devices, non-transitory computer readable storage media, or processes of the disclosure, a high TMB comprises a TMB of at least about 10 mut/Mb.

In some embodiments of any of the methods, systems, devices, non-transitory computer readable storage media, or processes of the disclosure, a genomic profile for the sample is generated based at least in part on detecting LOH of one or more HLA-I genes, or LOH of one or more HLA-I genes and a high TMB, in the sample.

In some embodiments, a genomic profile comprises the presence or absence of one or more genomic alterations in the sample, e.g., LOH of one or more HLA-I genes, or LOH of one or more HLA-I genes and a high TMB. In some embodiments, a genomic profile indicates the presence or absence of one or more genomic alterations in the sample, e.g., LOH of one or more HLA-I genes, or LOH of one or more HLA-I genes and a high TMB. In some embodiments, a genomic profile comprises information on the presence or absence of one or more genomic alterations in the sample, e.g., LOH of one or more HLA-I genes, or LOH of one or more HLA-I genes and a high TMB. For example, in some embodiments, the genomic profile comprises/indicates/comprises information on presence or absence of: (1) LOH of one or more HLA-I genes; (2) TMB (e.g., a specific value for TMB, or an indication of high TMB, low TMB, or the absence of high TMB); and/or (3) presence or absence of mutations in one or more additional genes, e.g., a panel of known oncogenes and/or tumor suppressors. In some embodiments, the genomic profile is obtained from a genomic profiling assay (such as a cancer- or tumor-related genomic profiling assay), e.g., as obtained using any of the sequencing methodologies described herein. In some embodiments, the genomic profile includes information from whole-genome or whole-exome sequencing. In some embodiments, the genomic profile includes information from targeted sequencing. In some embodiments, the genomic profile includes information from NGS. In some embodiments, the genomic profile comprises/indicates/comprises information on presence or absence of mutations such as short variant alterations (e.g., a base substitution, insertion, or deletion), copy-number alterations (e.g., an amplification or a homozygous deletion), and/or rearrangements (e.g., a gene fusion or other genomic or chromosomal rearrangement). In some embodiments, the genomic profile comprises/indicates/comprises information on presence or absence of TMB, or a TMB is calculated based in part on information on one or more mutations (or mutation rate, or type of mutation) detected as part of obtaining the genomic profile. In some embodiments, the genomic profile comprises/indicates/comprises information on presence or absence of HLA-I LOH, or HLA-I LOH is detected based in part on information on one or more mutations detected as part of obtaining the genomic profile.

### Tumor Mutational Burden

In some embodiments, the methods provided herein comprise acquiring knowledge of or detecting the level of tumor mutational burden in a cancer of the disclosure, e.g., a squamous cell cancer or NSCLC.

As demonstrated herein, LOH of one or more HLA-I genes and a high tumor mutational burden together in squamous cell cancer or NSCLC, e.g., squamous cell lung cancer or squamous NSCLC, may be predictive of increased overall survival, increased progression-free survival, increased probability of greater survival, and/or increased likelihood of response to immune checkpoint inhibitor therapy, e.g., as compared to squamous cell cancer or NSCLC (e.g., squamous cell lung cancer or squamous NSCLC) without a high tumor mutational burden (e.g., and with LOH of an HLA-I gene); without a high tumor mutational burden and without LOH of an HLA-I gene; or with a high tumor mutational burden (e.g., and without LOH of an HLA-I gene). See, e.g., Example 1. Accordingly, in some embodiments, provided herein are methods that comprise acquiring knowledge of or detecting a LOH of one or more HLA-I genes and a high tumor mutational burden in a squamous cell cancer or NSCLC. In some embodiments, a cancer of the disclosure, e.g., a squamous cell cancer or NSCLC, has a somatic LOH of one or more HLA-I genes, e.g., one or more of HLA-A, HLA-B or HLA-C, and a high tumor mutational burden. In some embodiments, high tumor mutational burden refers to a tumor mutational burden of greater than or equal to 10 mutations/Mb.

In some embodiments, acquiring knowledge of or detecting the level of tumor mutational burden in a cancer of the disclosure comprises measuring the level of tumor mutational burden in a sample, e.g., in a sample from a cancer or a tumor, obtained from an individual.

In some embodiments, tumor mutational burden is assessed in sample from an individual, such as sample described herein. In some embodiments, the sample from the individual comprises fluid, cells, or tissue. In some embodiments, the sample from the individual comprises a tumor biopsy or a circulating tumor cell. In some embodiments, the sample from the individual comprises nucleic acids. In some embodiments, the sample from the individual comprises mRNA, DNA, circulating tumor DNA, cell-free DNA, or cell-free RNA.

In some embodiments, tumor mutational burden is measured using any suitable method known in the art. For example, tumor mutational burden may be measured using whole-exome sequencing (WES), next-generation sequencing, whole genome sequencing, gene-targeted sequencing, or sequencing of a panel of genes, e.g., panels including cancer-related genes. See, e.g., Melendez et al., Transl Lung Cancer Res (2018) 7(6):661-667. In some embodiments, tumor mutational burden is measured using gene-targeted sequencing, e.g., using a nucleic acid hybridization-capture method, e.g., coupled with sequencing. See, e.g., Fancello et al., J Immunother Cancer (2019) 7:183.

In some embodiments, tumor mutational burden is measured according to the methods provided in WO2017151524A1, which is hereby incorporated by reference in its entirety. In some embodiments, tumor mutational burden is measured according to the methods described in Montesion, M., et al., Cancer Discovery (2021) 11(2):282-92.

In some embodiments, tumor mutational burden is assessed based on the number of non-driver somatic coding mutations/megabase (mut/Mb) of genome sequenced.

In some embodiments, tumor mutational burden is measured in the sample by whole exome sequencing. In some embodiments, tumor mutational burden is measured in the sample using next-generation sequencing. In some embodiments, tumor mutational burden is measured in the sample using whole genome sequencing. In some embodiments, tumor mutational burden is measured in the sample by gene-targeted sequencing. In some embodiments, tumor mutational burden is measured on between about 0.8 Mb and about 1.3 Mb of sequenced DNA. In some embodiments, tumor mutational burden is measured on any of about 0.8 Mb, about 0.81 Mb, about 0.82 Mb, about 0.83 Mb, about 0.84 Mb, about 0.85 Mb, about 0.86 Mb, about 0.87 Mb, about 0.88 Mb, about 0.89 Mb, about 0.9 Mb, about 0.91 Mb, about 0.92 Mb, about 0.93 Mb, about 0.94 Mb, about 0.95 Mb, about 0.96 Mb, about 0.97 Mb, about 0.98 Mb, about 0.99 Mb, about 1 Mb, about 1.01 Mb, about 1.02 Mb, about 1.03 Mb, about 1.04 Mb, about 1.05 Mb, about 1.06 Mb, about 1.07 Mb, about 1.08 Mb, about 1.09 Mb, about 1.1 Mb, about 1.2 Mb, or about 1.3 Mb of sequenced DNA. In some embodiments, tumor mutational burden is measured on about 0.8 Mb of sequenced DNA. In some embodiments, tumor mutational burden is measured on between about 0.83 Mb and about 1.14 Mb of sequenced DNA. In some embodiments, tumor mutational burden is measured on up to about 1.24 Mb of sequenced DNA. In some embodiments, tumor mutational burden is measured on up to about 1.1 Mb of sequenced DNA. In some embodiments, tumor mutational burden is measured on about 0.79 Mb of sequenced DNA.

In some embodiments, a cancer of the disclosure, e.g., a squamous cell cancer or NSCLC, has a tumor mutational burden of less than about 10 mut/Mb, e.g., any of about 9.9 mut/Mb, about 9.8 mut/Mb, about 9.6 mut/Mb, about 9.4 mut/Mb, about 9.2 mut/Mb, about 9 mut/Mb, about 8.8 mut/Mb, about 8.6 mut/Mb, about 8.4 mut/Mb, about 8.2 mut/Mb, about 8 mut/Mb, about 7.8 mut/Mb, about 7.6 mut/Mb, about 7.4 mut/Mb, about 7.2 mut/Mb, about 7 mut/Mb, about 6.8 mut/Mb, about 6.6 mut/Mb, about 6.4 mut/Mb, about 6.2 mut/Mb, about 6 mut/Mb, about 5.8 mut/Mb, about 5.6 mut/Mb, about 5.4 mut/Mb, about 5.2 mut/Mb, about 5 mut/Mb, about 4.8 mut/Mb, about 4.6 mut/Mb, about 4.4 mut/Mb, about 4.2 mut/Mb, about 4 mut/Mb, about 3.8 mut/Mb, about 3.6 mut/Mb, about 3.4 mut/Mb, about 3.2 mut/Mb, about 3 mut/Mb, about 2.8 mut/Mb, about 2.6 mut/Mb, about 2.4 mut/Mb, about 2.2 mut/Mb, about 2 mut/Mb, about 1.8 mut/Mb, about 1.6 mut/Mb, about 1.4 mut/Mb, about 1.2 mut/Mb, about 1 mut/Mb, about 0.8 mut/Mb, about 0.6 mut/Mb, about 0.4 mut/Mb, about 0.2 mut/Mb, or less.

In some embodiments, a cancer of the disclosure, e.g., a squamous cell cancer or NSCLC, has a high tumor mutational burden, e.g., of at least about 10 mut/Mb. In some embodiments, the cancer has a tumor mutational burden of at least about 10 mut/Mb. In some embodiments, the cancer has a tumor mutational burden of at least about 20 mut/Mb. In some embodiments, the cancer has a tumor mutational burden of any of between about 10 mut/Mb and about 15 mut/Mb, between about 15 mut/Mb and about 20 mut/Mb, between about 20 mut/Mb and about 25 mut/Mb, between about 25 mut/Mb and about 30 mut/Mb, between about 30 mut/Mb and about 35 mut/Mb, between about 35 mut/Mb and about 40 mut/Mb, between about 40 mut/Mb and about 45 mut/Mb, between about 45 mut/Mb and about 50 mut/Mb, between about 50 mut/Mb and about 55 mut/Mb, between about 55 mut/Mb and about 60 mut/Mb, between about 60 mut/Mb and about 65 mut/Mb, between about 65 mut/Mb and about 70 mut/Mb, between about 70 mut/Mb and about 75 mut/Mb, between about 75 mut/Mb and about 80 mut/Mb, between about 80 mut/Mb and about 85 mut/Mb, between about 85 mut/Mb and about 90 mut/Mb, between about 90 mut/Mb and about 95 mut/Mb, or between about 95 mut/Mb and about 100 mut/Mb. In some embodiments, the cancer has a tumor mutational burden of any of between about 100 mut/Mb and about 110 mut/Mb, between about 110 mut/Mb and about 120 mut/Mb, between about 120 mut/Mb and about 130 mut/Mb, between about 130 mut/Mb and about 140 mut/Mb, between about 140 mut/Mb and about 150 mut/Mb, between about 150 mut/Mb and about 160 mut/Mb, between about 160 mut/Mb and about 170 mut/Mb, between about 170 mut/Mb and about 180 mut/Mb, between about 180 mut/Mb and about 190 mut/Mb, between about 190 mut/Mb and about 200 mut/Mb, between about 210 mut/Mb and about 220 mut/Mb, between about 220 mut/Mb and about 230 mut/Mb, between about 230 mut/Mb and about 240 mut/Mb, between about 240 mut/Mb and about 250 mut/Mb, between about 250 mut/Mb and about 260 mut/Mb, between about 260 mut/Mb and about 270 mut/Mb, between about 270 mut/Mb and about 280 mut/Mb, between about 280 mut/Mb and about 290 mut/Mb, between about 290 mut/Mb and about 300 mut/Mb, between about 300 mut/Mb and about 310 mut/Mb, between about 310 mut/Mb and about 320 mut/Mb, between about 320 mut/Mb and about 330 mut/Mb, between about 330 mut/Mb and about 340 mut/Mb, between about 340 mut/Mb and about 350 mut/Mb, between about 350 mut/Mb and about 360 mut/Mb, between about 360 mut/Mb and about 370 mut/Mb, between about 370 mut/Mb and about 380 mut/Mb, between about 380 mut/Mb and about 390 mut/Mb, between about 390 mut/Mb and about 400 mut/Mb, or more than 400 mut/Mb. In some embodiments, the cancer has a TMB of at least about 100 mut/Mb, at least about 110 mut/Mb, at least about 120 mut/Mb, at least about 130 mut/Mb, at least about 140 mut/Mb, at least about 150 mut/Mb, or more.

In some embodiments, measuring tumor mutational burden comprises assessing mutations in a sample derived from a cancer, e.g., a squamous cell cancer or NSCLC, in an individual. In some embodiments, measuring tumor mutational burden comprises assessing mutations in a sample derived from a cancer, e.g., a squamous cell cancer or NSCLC, in an individual and in a matched normal sample, e.g., a sample from the individual derived from a tissue or other source that is free of the cancer.

In some embodiments, tumor mutational burden is obtained from a plurality of sequence reads, e.g., a plurality of sequence reads obtained by sequencing nucleic acids corresponding to at least a portion of a genome (such as from an enriched or unenriched sample). In some embodiments, tumor mutational burden is determined based on the number of non-driver somatic coding mutations per megabase of genome sequenced.

In some embodiments, any of the methods of the present disclosure comprise acquiring knowledge of LOH of one or more HLA-I genes (e.g., in a sample obtained from an individual) and acquiring knowledge of tumor mutational burden (e.g., in a sample obtained from an individual). In some embodiments, any of the methods of the present disclosure comprise detecting LOH of one or more HLA-I genes (e.g., in a sample obtained from an individual) and acquiring knowledge of tumor mutational burden (e.g., in a sample obtained from an individual). In some embodiments, any of the methods of the present disclosure comprise acquiring knowledge of LOH of one or more HLA-I genes (e.g., in a sample obtained from an individual) and detecting or determining tumor mutational burden (e.g., in a sample obtained from an individual). In some embodiments, any of the methods of the present disclosure comprise detecting LOH of one or more HLA-I genes (e.g., in a sample obtained from an individual) and detecting or determining tumor mutational burden (e.g., in a sample obtained from an individual).

In some embodiments of any of the methods of the disclosure, the samples used to detect/determine LOH of one or more HLA-I genes and tumor mutational burden are the same. In some embodiments of any of the methods of the disclosure, the samples used to detect/determine LOH of one or more HLA-I genes and tumor mutational burden are different. In some embodiments of any of the methods of the disclosure, LOH of one or more HLA-I genes and tumor mutational burden are detected/determined in the same sample. In some embodiments of any of the methods of the disclosure, LOH of one or more HLA-I genes and tumor mutational burden are detected/determined in different samples.

### PD-L1 Expression

In some embodiments, a squamous cell cancer or NSCLC of the disclosure is PD-L1 positive. In some embodiments, the methods provided herein comprise acquiring knowledge of or detecting the level of PD-L1 expression in a squamous cell cancer or NSCLC of the disclosure. In some embodiments, acquiring knowledge of or detecting the level of PD-L1 expression in a squamous cell cancer or NSCLC of the disclosure comprises measuring PD-L1 expression in a sample, e.g., in a sample from a squamous cell cancer or NSCLC, obtained from an individual.

Any suitable method for measuring PD-L1 expression in a sample from an individual may be used. For example, the level of PD-L1 expression may be measured using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), MassARRAY, proteomics (e.g., mass spectrometry), quantitative blood based assays (as for example serum ELISA), biochemical enzymatic activity assays, in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”). Multiplexed immunoassays such as those available from Rules Based Medicine or Meso Scale Discovery (“MSD”) may also be used.

In some embodiments, PD-L1 expression in a sample from an individual is measured based on the level of PD-L1 mRNA in the sample. Any suitable method for measuring mRNA expression in a sample from an individual may be used. For example, the level of PD-L1 mRNA expression may be measured using in situ hybridization, Northern analysis, polymerase chain reaction (“PCR”) including quantitative real time PCR (qRT-PCR) and other amplification-based methods, RNA-sequencing (RNA-seq), FISH, microarray analysis, gene expression profiling, and/or serial analysis of gene expression (“SAGE”).

In some embodiments, PD-L1 expression in a sample from an individual is measured based on the level of PD-L1 protein in the sample. Any suitable method for measuring protein expression in a sample from an individual may be used. For example, the level of PD-L1 protein expression may be measured using immunohistochemistry (IHC), Western blot analysis, immunoprecipitation, molecular binding assays, enzyme-linked immunosorbent assay (ELISA), enzyme-linked immunofiltration assay (ELIFA), fluorescence activated cell sorting (FACS), proteomics (e.g., mass spectrometry), quantitative blood based assays (as for example serum ELISA), biochemical enzymatic activity assays, or multiplexed immunoassays such as those available from Rules Based Medicine or Meso Scale Discovery (“MSD”).

In some embodiments, PD-L1 expression is measured by immunohistochemistry using commercially available antibody clones 22C3 (Dako/Agilent) or SP142 (Ventana), e.g., according to the methods described in Montesion, M., et al., Cancer Discovery (2021) 11(2):282-92.

In some embodiments, a cancer provided herein, e.g., a squamous cell cancer or NSCLC, is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs), e.g., in a sample from an individual, express PD-L1 protein and/or PD-L1 mRNA (e.g., are positive for PD-L1 protein and/or PD-L1 mRNA). In some embodiments, a cancer provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs), e.g., in a sample from an individual, express PD-L1 protein and/or PD-L1 mRNA (e.g., are positive for PD-L1 protein and/or PD-L1 mRNA). In some embodiments, a cancer provided herein is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor cells, e.g., in a sample from an individual, express PD-L1 protein and/or PD-L1 mRNA (e.g., are positive for PD-L1 protein and/or PD-L1 mRNA). In some embodiments, the sample is obtained from the cancer, such as a squamous cell cancer or NSCLC.

In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) and/or tumor cells (TCs) in the sample express PD-L1 protein and/or PD-L1 mRNA (e.g., are positive for PD-L1 protein and/or PD-L1 mRNA). In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor infiltrating immune cells (ICs) in the sample express PD-L1 protein and/or PD-L1 mRNA (e.g., are positive for PD-L1 protein and/or PD-L1 mRNA). In some embodiments, a sample from an individual is determined to be positive for PD-L1 if at least about 1% (e.g., any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%) of tumor cells in the sample express PD-L1 protein and/or PD-L1 mRNA (e.g., are positive for PD-L1 protein and/or PD-L1 mRNA). In some embodiments, the sample is obtained from the cancer, such as a squamous cell cancer or NSCLC.

In some embodiments, the level of PD-L1 protein and/or PD-L1 mRNA is assessed in a sample from an individual, such as a sample described herein. In some embodiments, the sample from the individual comprises fluid, cells, or tissue. In some embodiments, the sample from the individual comprises a tumor biopsy or a circulating tumor cell. In some embodiments, the sample is obtained or derived from a cancer, e.g., a squamous cell cancer or NSCLC.

In some embodiments of any of the methods provided herein, a sample from an individual, e.g., an individual having a squamous cell cancer or NSCLC, is determined to be PD-L1-negative if less than 1% of tumor cells in the sample express PD-L1. In some embodiments of any of the methods provided herein, a sample from an individual, e.g., an individual having a squamous cell cancer or NSCLC, is determined to be PD-L1 positive if at least about 1% of tumor cells in the sample express PD-L1.

In some embodiments, the level of PD-L1 protein expression is measured using an immunohistochemistry assay. In some embodiments, the level of PD-L1 protein expression is measured using a VENTANA PD-L1 assay (SP142). In some embodiments, the level of PD-L1 protein expression is determined based on PD-L1 expression in tumor infiltrating immune cells (ICs) and/or tumor cells (TCs). Additional information about the VENTANA SP142 assay may be found in the website: www[dot]accessdata[dot]fda[dot]gov/cdrh_docs/pdf16/PI60002c.pdf.

In some embodiments, the level of PD-L1 protein expression is determined based on PD-L1 tumor cell expression using an immunohistochemistry assay, such as a DAKO 22C3 assay. In some embodiments, the level of PD-L1 protein expression is assessed based on a tumor proportion score (TPS). The TPS is the percentage of tumor cells showing partial or complete PD-L1 membrane staining (e.g., at a ≥1+ intensity on a 0, 1+, 2+, and 3 scale) relative to all tumor cells present in the sample. In some embodiments, the TPS is calculated as: the number of PD-L1-positive tumor cells/Total number of PD-L1-positive tumor cells+Total number of PD-L1-negative tumor cells. A PD-L1 low positive status refers to a TPS of between 1% and 49%, PD-L1 high positive status refers to a TPS of 50% or greater, and a PD-L1 negative status refers to a TPS of less than 1%. In some embodiments, a cancer of the disclosure is determined to be PD-L1 positive if it has PD-L1 low positive status or a PD-L1 high positive status. In some embodiments, a cancer of the disclosure is PD-L1 positive (e.g., the cancer is determined have a TPS of any of at least about 1%, at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or 100%, in a sample obtained from an individual having the cancer). In some embodiments, a cancer of the disclosure is PD-L1 low positive (e.g., the cancer is determined have a TPS of any of about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, or about 49%, in a sample obtained from an individual having the cancer). In some embodiments, a cancer of the disclosure is PD-L1 high positive (e.g., the cancer is determined have a TPS of any of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%, in a sample obtained from an individual having the cancer). In some embodiments, a cancer of the disclosure is PD-L1 negative (e.g., the cancer is determined have a TPS of less than 1%, in a sample obtained from an individual having the cancer). Additional information about the DAKO 22C3 assay and the TPS score may be found, e.g., in the website: www[dot]agilent[dot]com/cs/library/usermanuals/public/29158_pd-11-ihc-22C3-pharmdx-nsclc-interpretation-manual.pdf.

### Reporting

In some embodiments, the methods provided herein comprise generating a report, and/or providing a report to party.

In some embodiments, a report according to the present disclosure comprises information about one or more of: a somatic LOH of one or more HLA-I genes, or a somatic LOH of one or more HLA-I genes and high TMB; a cancer of the disclosure, e.g., a squamous cell cancer or NSCLC, optionally comprising a somatic LOH of one or more HLA-I genes, or a somatic LOH of one or more HLA-I genes and high TMB; and a treatment, a therapy, or one or more treatment options for an individual having a cancer of the disclosure, e.g., a squamous cell cancer or NSCLC, optionally comprising a somatic LOH of one or more HLA-I genes, or a somatic LOH of one or more HLA-I genes and high TMB.

In some embodiments, a report according to the present disclosure comprises information about the presence or absence of a somatic LOH of one or more HLA-I genes, or a somatic LOH of one or more HLA-I genes and high TMB in a sample obtained from an individual, such as an individual having a cancer, e.g., a squamous cell cancer or NSCLC. In one embodiment, a report according to the present disclosure indicates that a somatic LOH of one or more HLA-I genes, or a somatic LOH of one or more HLA-I genes and high TMB are present in a sample obtained from the individual. In one embodiment, a report according to the present disclosure indicates that a somatic LOH of one or more HLA-I genes, or a somatic LOH of one or more HLA-I genes and high TMB are not present in a sample obtained from the individual. In one embodiment, a report according to the present disclosure indicates that a somatic LOH of one or more HLA-I genes, or a somatic LOH of one or more HLA-I genes and high TMB have been detected in a sample obtained from the individual. In one embodiment, a report according to the present disclosure indicates that a somatic LOH of one or more HLA-I genes, or a somatic LOH of one or more HLA-I genes and high TMB have not been detected in a sample obtained from the individual. In some embodiments, the report comprises an identifier for the individual from which the sample was obtained.

In some embodiments, the report includes information on the role of a somatic LOH of one or more HLA-I genes, or a somatic LOH of one or more HLA-I genes and high TMB, in disease, such as in cancer, e.g. in squamous cell cancer or NSCLC. Such information can include one or more of: information on prognosis of a cancer, e.g., a squamous cell cancer or NSCLC, or a squamous cell cancer or NSCLC comprising a somatic LOH of one or more HLA-I genes, or a somatic LOH of one or more HLA-I genes and high TMB; information on resistance of the cancer to one or more treatments; information on potential or suggested therapeutic options (e.g., an anti-cancer therapy provided herein, e.g., an immune checkpoint inhibitor, or a treatment selected or identified according to the methods provided herein); or information on therapeutic options that should be avoided. In some embodiments, the report includes information on the likely effectiveness, acceptability, and/or advisability of applying a therapeutic option (e.g., an anti-cancer therapy provided herein, such as an immune checkpoint inhibitor, or a treatment selected or identified according to the methods provided herein) to an individual having a cancer, e.g., a squamous cell cancer or NSCLC, or a squamous cell cancer or NSCLC comprising a somatic LOH of one or more HLA-I genes, or a somatic LOH of one or more HLA-I genes and high TMB, and identified in the report. In some embodiments, the report includes information or a recommendation on the administration of a treatment (e.g., an anti-cancer therapy provided herein, such as an immune checkpoint inhibitor, or a treatment selected or identified according to the methods provided herein). In some embodiments, the information or recommendation includes the dosage of the treatment and/or a treatment regimen (e.g., as a monotherapy, or in combination with other treatments, such as a second anti-cancer agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, e.g., a squamous cell cancer or NSCLC; detecting a somatic LOH of one or more HLA-I genes, or a somatic LOH of one or more HLA-I genes and high TMB in the sample, or acquiring knowledge of the presence of a somatic LOH of one or more HLA-I genes, or a somatic LOH of one or more HLA-I genes and high TMB in the sample; and generating a report. In some embodiments, a report generated according to the methods provided herein comprises one or more of: information about the presence or absence of a somatic LOH of one or more HLA-I genes, or a somatic LOH of one or more HLA-I genes and high TMB in the sample; an identifier for the individual from which the sample was obtained; information on the role of the somatic LOH of one or more HLA-I genes, or the somatic LOH of one or more HLA-I genes and high TMB in disease (e.g., such as in cancer, e.g., in a squamous cell cancer or NSCLC); information on prognosis, resistance, or potential or suggested therapeutic options (e.g., an anti-cancer therapy provided herein, such as an immune checkpoint inhibitor, or a treatment selected or identified according to the methods provided herein); information on the likely effectiveness, acceptability, or the advisability of applying a therapeutic option (e.g., an anti-cancer therapy provided herein, such as an immune checkpoint inhibitor, or a treatment selected or identified according to the methods provided herein) to the individual; a recommendation or information on the administration of a treatment (e.g., an anti-cancer therapy provided herein, such as an immune checkpoint inhibitor, or a treatment selected or identified according to the methods provided herein); or a recommendation or information on the dosage or treatment regimen of a treatment (e.g., an anti-cancer therapy provided herein, such as an immune checkpoint inhibitor, or a treatment selected or identified according to the methods provided herein), e.g., in combination with other treatments (e.g., a second anti-cancer therapy). In some embodiments, the report generated is a personalized cancer report.

A report according to the present disclosure may be in an electronic, web-based, or paper form. The report may be provided to an individual or a patient (e.g., an individual or a patient with a cancer, such as a squamous cell cancer or NSCLC, e.g., comprising a somatic LOH of one or more HLA-I genes, or a somatic LOH of one or more HLA-I genes and high TMB), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer, such as a squamous cell cancer or NSCLC, e.g., comprising a somatic LOH of one or more HLA-I genes, or a somatic LOH of one or more HLA-I genes and high TMB). In some embodiments, the report is provided or delivered to an individual or entity within any of about 1 day or more, about 7 days or more, about 14 days or more, about 21 days or more, about 30 days or more, about 45 days or more, or about 60 days or more from detecting a somatic LOH of one or more HLA-I genes, or a somatic LOH of one or more HLA-I genes and high TMB in a sample obtained from an individual (e.g., an individual having a cancer, such as a squamous cell cancer or NSCLC). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of a somatic LOH of one or more HLA-I genes, or a somatic LOH of one or more HLA-I genes and high TMB in a sample obtained from an individual (e.g., an individual having a cancer, such as a squamous cell cancer or NSCLC).

### Immune Checkpoint Inhibitors and Anti-Cancer Therapies

Certain aspects of the present disclosure relate to immune checkpoint inhibitors (ICIs). As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSFIR, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCN1 (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSF14, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments according to any of the embodiments described herein, the ICI comprises a PD-1 antagonist/inhibitor or a PD-L1 antagonist/inhibitor.

In some embodiments, the checkpoint inhibitor is a PD-L1 axis binding antagonist, e.g., a PD-1 binding antagonist, a PD-L1 binding antagonist, or a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “37-H,” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist/inhibitor is a molecule that inhibits the binding of PD-1 to its ligand binding partners. In a specific embodiment, the PD-1 ligand binding partners are PD-L1 and/or PD-L2. In another instance, a PD-L1 binding antagonist/inhibitor is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is MDX-1106 (nivolumab), MK-3475 (pembrolizumab, Keytruda®), cemiplimab, dostarlimab, MEDI-0680 (AMP-514), PDR001, REGN2810, MGA-012, JNJ-63723283, BI 754091, or BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANBO11; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IB1308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, Keytruda®), PF-06801591, cemiplimab (REGN-2810, REGEN2810), dostarlimab (TSR-042, ANBO11), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No. Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.S70, MPDL3280A (atezolizumab), MDX-1105, MEDI4736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist/inhibitor of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (1B1310, BMS-734016, MDX010, MDX-CTLA4, MED14736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, Keytruda®), cemiplimab, dostarlimab, MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MED14736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the immune checkpoint inhibitor is monovalent and/or monospecific. In some embodiments, the immune checkpoint inhibitor is multivalent and/or multispecific.

In some embodiments, the immune checkpoint inhibitor may be administered in combination with an immunoregulatory molecule or a cytokine. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3α (Lax), MIP-3β, Hcc-1, MPIF-1, MPIF-2, MCP-2, MCP-3, MCP-4, MCP-5, Eotaxin, Tarc, Elc, 1309, IL-8, GCP-2 Groa, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the methods provided herein comprise administering to an individual a treatment that comprises an immune checkpoint inhibitor (e.g., as described supra). In some embodiments, the methods provided herein comprise selecting/identifying a treatment or one or more treatment options for an individual, wherein the treatment or the one or more treatment options comprise an immune checkpoint inhibitor (e.g., as described supra). In some embodiments, the treatment or the one or more treatment options further comprise an additional anti-cancer therapy. In some embodiments, the additional anti-cancer therapy is an agent other than an ICI (e.g., as described infra), or a second ICI (e.g., as described supra).

In some embodiments, the anti-cancer therapy comprises a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, an anti-hormonal agent, a kinase inhibitor, a peptide, a gene therapy, a vaccine, a platinum-based chemotherapeutic agent, an immunotherapy, a growth inhibitory agent, a cytotoxic agent, an antimetabolite chemotherapeutic agent, or any combination thereof.

In some embodiments, the anti-cancer therapy comprises a chemotherapy. In some embodiments, the methods provided herein comprise administering to the individual a chemotherapy, e.g., in combination with another anti-cancer therapy such as an immune checkpoint inhibitor. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein tansferase inhibitors, transplatinum, and pharmaceutically acceptable salts, acids, or derivatives of any of the above.

Some non-limiting examples of chemotherapeutic drugs which can be combined with anti-cancer therapies of the present disclosure, such as an immune checkpoint inhibitor, are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, the anti-cancer therapy comprises a kinase inhibitor. In some embodiments, the methods provided herein comprise administering to the individual a kinase inhibitor, e.g., in combination with another anti-cancer therapy such as an immune checkpoint inhibitor. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR3, FGFR4, CSF1R, c-Met, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU11248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises an anti-angiogenic agent. In some embodiments, the methods provided herein comprise administering to the individual an anti-angiogenic agent, e.g., in combination with another anti-cancer therapy such as an immune checkpoint inhibitor. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-a platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v β3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy. In some embodiments, the methods provided herein comprise administering to the individual an anti-DNA repair therapy, e.g., in combination with another anti-cancer therapy such as an immune checkpoint inhibitor. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer. In some embodiments, the methods provided herein comprise administering to the individual a radiosensitizer, e.g., in combination with another anti-cancer therapy such as an immune checkpoint inhibitor. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent. In some embodiments, the methods provided herein comprise administering to the individual an anti-inflammatory agent, e.g., in combination with another anti-cancer therapy such as an immune checkpoint inhibitor. In some embodiments, the anti-inflammatory agent is an agent that blocks, inhibits, or reduces inflammation or signaling from an inflammatory signaling pathway In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (Humira®), certolizumab pegol (Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, Ala-Cort®, Hydrocort Acetate®, hydrocortone phosphate Lanacort®, Solu-Cortef®), decadron (dexamethasone, dexamethasone acetate, dexamethasone sodium phosphate, Dexasone®, Diodex®, Hexadrol®, Maxidex®), methylprednisolone (6-methylprednisolone, methylprednisolone acetate, methylprednisolone sodium succinate, Duralone®, Medralone®, Medrol®, M-Prednisol®, Solu-Medrol®), prednisolone (Delta-Cortef®, ORAPRED®, Pediapred®, Prezone®), and prednisone (Deltasone®, Liquid Pred®, Meticorten®, Orasone®), and bisphosphonates (e.g., pamidronate (Aredia®), and zoledronic acid (Zometac®).

In some embodiments, the anti-cancer therapy comprises an anti-hormonal agent. In some embodiments, the methods provided herein comprise administering to the individual an anti-hormonal agent, e.g., in combination with another anti-cancer therapy such as an immune checkpoint inhibitor. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent. In some embodiments, the methods provided herein comprise administering to the individual an antimetabolite chemotherapeutic agent, e.g., in combination with another anti-cancer therapy such as an immune checkpoint inhibitor. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOMED), azocytosine, deoxycytosine, pyridmidene, fludarabine (FLUDARA®), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the anti-cancer therapy comprises a platinum-based chemotherapeutic agent. In some embodiments, the methods provided herein comprise administering to the individual a platinum-based chemotherapeutic agent, e.g., in combination with another anti-cancer therapy such as an immune checkpoint inhibitor. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some embodiments, the anti-cancer therapy comprises a heat shock protein (HSP) inhibitor, a MYC inhibitor, an HDAC inhibitor, an immunotherapy, a neoantigen, a vaccine, or a cellular therapy. In some embodiments, the anti-cancer therapy includes one or more of a chemotherapy, a VEGF inhibitor, an Integrin 03 inhibitor, a statin, an EGFR inhibitor, an mTOR inhibitor, a PI3K inhibitor, a MAPK inhibitor, or a CDK4/6 inhibitor.

In some embodiments, the anti-cancer therapy comprises a kinase inhibitor. In some embodiments, the methods provided herein comprise administering to the individual a kinase inhibitor, e.g., in combination with another anti-cancer therapy such as an immune checkpoint inhibitor. In some embodiments, the kinase inhibitor is crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). Additional examples of ALK kinase inhibitors that may be used according to any of the methods provided herein are described in examples 3-39 of WO2005016894, which is incorporated herein by reference.

In some embodiments, the anti-cancer therapy comprises a heat shock protein (HSP) inhibitor. In some embodiments, the methods provided herein comprise administering to the individual an HSP inhibitor, e.g., in combination with another anti-cancer therapy such as an immune checkpoint inhibitor. In some embodiments, the HSP inhibitor is a Pan-HSP inhibitor, such as KNK423. In some embodiments, the HSP inhibitor is an HSP70 inhibitor, such as cmHsp70.1, quercetin, VER155008, or 17-AAD. In some embodiments, the HSP inhibitor is a HSP90 inhibitor. In some embodiments, the HSP90 inhibitor is 17-AAD, Debio0932, ganetespib (STA-9090), retaspimycin hydrochloride (retaspimycin, IPI-504), AUY922, alvespimycin (KOS-1022, 17-DMAG), tanespimycin (KOS-953, 17-AAG), DS 2248, or AT13387 (onalespib). In some embodiments, the HSP inhibitor is an HSP27 inhibitor, such as Apatorsen (OGX-427).

In some embodiments, the anti-cancer therapy comprises a MYC inhibitor. In some embodiments, the methods provided herein comprise administering to the individual a MYC inhibitor, e.g., in combination with another anti-cancer therapy such as an immune checkpoint inhibitor. In some embodiments, the MYC inhibitor is MYCi361 (NUCC-0196361), MYCi975 (NUCC-0200975), Omomyc (dominant negative peptide), ZINC16293153 (Min9), 10058-F4, JKY-2-169, 7594-0035, or inhibitors of MYC/MAX dimerization and/or MYC/MAX/DNA complex formation.

In some embodiments, the anti-cancer therapy comprises a histone deacetylase (HDAC) inhibitor. In some embodiments, the methods provided herein comprise administering to the individual an HDAC inhibitor, e.g., in combination with another anti-cancer therapy such as an immune checkpoint inhibitor. In some embodiments, the HDAC inhibitor is belinostat (PXD101, Beleodaq®), SAHA (vorinostat, suberoylanilide hydroxamine, Zolinza®), panobinostat (LBH589, LAQ-824), ACY1215 (Rocilinostat), quisinostat (JNJ-26481585), abexinostat (PCI-24781), pracinostat (SB939), givinostat (ITF2357), resminostat (4SC-201), trichostatin A (TSA), MS-275 (etinostat), Romidepsin (depsipeptide, FK228), MGCD0103 (mocetinostat), BML-210, CAY10603, valproic acid, MC1568, CUDC-907, CI-994 (Tacedinaline), Pivanex (AN-9), AR-42, Chidamide (CS055, HBI-8000), CUDC-101, CHR-3996, MPTOE028, BRD8430, MRLB-223, apicidin, RGFP966, BG45, PCI-34051, C149 (NCC149), TMP269, Cpd2, T247, T326, LMK235, CIA, HPOB, Nexturastat A, Befexamac, CBHA, Phenylbutyrate, MC1568, SNDX275, Scriptaid, Merck60, PX089344, PX105684, PX117735, PX117792, PX117245, PX105844, compound 12 as described by Li et al., Cold Spring Harb Perspect Med (2016) 6(10):a026831, or PX117445.

In some embodiments, the anti-cancer therapy comprises a VEGF inhibitor. In some embodiments, the methods provided herein comprise administering to the individual a VEGF inhibitor, e.g., in combination with another anti-cancer therapy such as an immune checkpoint inhibitor. In some embodiments, the VEGF inhibitor is Bevacizumab (Avastin®), BMS-690514, ramucirumab, pazopanib, sorafenib, sunitinib, golvatinib, vandetanib, cabozantinib, levantinib, axitinib, cediranib, tivozanib, lucitanib, semaxanib, nindentanib, regorafinib, or aflibercept.

In some embodiments, the anti-cancer therapy comprises an integrin β3 inhibitor. In some embodiments, the methods provided herein comprise administering to the individual an integrin β3 inhibitor, e.g., in combination with another anti-cancer therapy such as an immune checkpoint inhibitor. In some embodiments, the integrin β3 inhibitor is anti-avb3 (clone LM609), cilengitide (EMD121974, NSC, 707544), an siRNA, GLPG0187, MK-0429, CNTO95, TN-161, etaracizumab (MEDI-522), intetumumab (CNT095) (anti-alphaV subunit antibody), abituzumab (EMD 525797/DI17E6) (anti-alphaV subunit antibody), JSM6427, SJ749, BCH-15046, SCH221153, or SC56631. In some embodiments, the anti-cancer therapy comprises an αIIbβ3 integrin inhibitor. In some embodiments, the methods provided herein comprise administering to the individual an αIIbβ3 integrin inhibitor, e.g., in combination with another anti-cancer therapy such as an immune checkpoint inhibitor. In some embodiments, the αIIbβ3 integrin inhibitor is abciximab, eptifibatide (Integrilin®), or tirofiban (Aggrastat®).

In some embodiments, the anti-cancer therapy comprises a statin or a statin-based agent. In some embodiments, the methods provided herein comprise administering to the individual a statin or a statin-based agent, e.g., in combination with another anti-cancer therapy such as an immune checkpoint inhibitor. In some embodiments, the statin or statin-based agent is simvastatin, atorvastatin, fluvastatin, pitavastatin, pravastatin, rosuvastatin, or cerivastatin.

In some embodiments, the anti-cancer therapy comprises an mTOR inhibitor. In some embodiments, the methods provided herein comprise administering to the individual an mTOR inhibitor, e.g., in combination with another anti-cancer therapy such as an immune checkpoint inhibitor. In some embodiments, the mTOR inhibitor is temsirolimus (CCI-779), KU-006379, PP242, Torin1, Torin2, ICSN3250, Rapalink-1, CC-223, sirolimus (rapamycin), everolimus (RAD001), dactosilib (NVP-BEZ235), GSK2126458, WAY-001, WAY-600, WYE-687, WYE-354, SF1126, XL765, INK128 (MLN012), AZD8055, OSI027, AZD2014, or AP-23573.

In some embodiments, the anti-cancer therapy comprises a PI3K inhibitor. In some embodiments, the methods provided herein comprise administering to the individual a PI3K inhibitor, e.g., in combination with another anti-cancer therapy such as an immune checkpoint inhibitor. In some embodiments, the PI3K inhibitor is GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDCO0032, IPI145, INK1117 (MLN1117), SAR260301, KIN-193 (AZD6482), duvelisib, GS-9820, GSK2636771, GDC-0980, AMG319, pazobanib, or alpelisib (BYL719, Piqray).

In some embodiments, the anti-cancer therapy comprises a MAPK inhibitor. In some embodiments, the methods provided herein comprise administering to the individual a MAPK inhibitor, e.g., in combination with another anti-cancer therapy such as an immune checkpoint inhibitor. In some embodiments, the MAPK inhibitor is SB203580, SKF-86002, BIRB-796, SC-409, RJW-67657, BIRB-796, VX-745, R03201195, SB-242235, or MW181.

In some embodiments, the anti-cancer therapy comprises a CDK4/6 inhibitor. In some embodiments, the methods provided herein comprise administering to the individual a CDK4/6 inhibitor, e.g., in combination with another anti-cancer therapy such as an immune checkpoint inhibitor. In some embodiments, the CDK4/6 inhibitor is ribociclib (Kisqali®, LEE011), palbociclib (PD0332991, Ibrance®), or abemaciclib (LY2835219).

In some embodiments, the anti-cancer therapy comprises an EGFR inhibitor. In some embodiments, the methods provided herein comprise administering to the individual an EGFR inhibitor, e.g., in combination with another anti-cancer therapy such as an immune checkpoint inhibitor. In some embodiments, the EGFR inhibitor is cetuximab, panitumumab, lapatinib, gefitinib, vandetanib, dacomitinib, icotinib, osimertinib (AZD9291), afatanib, olmutinib, EGF816 (nazartinib), avitinib (AC00010), rociletinib (CO-1686), BMS-690514, YH5448, PF-06747775, ASP8273, PF299804, AP26113, or erlotinib. In some embodiments, the EGFR inhibitor is gefitinib or cetuximab.

In some embodiments, the anti-cancer therapy comprises a cancer immunotherapy, such as a cancer vaccine, cell-based therapy, T cell receptor (TCR)-based therapy, adjuvant immunotherapy, cytokine immunotherapy, and oncolytic virus therapy. In some embodiments, the methods provided herein comprise administering to the individual a cancer immunotherapy, such as a cancer vaccine, cell-based therapy, T cell receptor (TCR)-based therapy, adjuvant immunotherapy, cytokine immunotherapy, and oncolytic virus therapy, e.g., in combination with another anti-cancer therapy such as an immune checkpoint inhibitor. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen expressed by a cancer of the disclosure. The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine. A range of cancer vaccines have been tested that employ different approaches to promoting an immune response against a cancer (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et ah, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104: 14-21), 2013). In some embodiments, such cancer vaccines augment the anti-cancer response.

In some embodiments, the cancer vaccine comprises a polynucleotide that encodes a neoantigen, e.g., a neoantigen expressed by a cancer of the disclosure. In some embodiments, the cancer vaccine comprises DNA or RNA that encodes a neoantigen. In some embodiments, the cancer vaccine comprises a polynucleotide that encodes a neoantigen. In some embodiments, the cancer vaccine further comprises one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543), prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and barnaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or BSR. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some embodiments, the anti-cancer therapy comprises a nucleic acid molecule, such as a dsRNA, an siRNA, or an shRNA. In some embodiments, the methods provided herein comprise administering to the individual a nucleic acid molecule, such as a dsRNA, an siRNA, or an shRNA, e.g., in combination with another anti-cancer therapy. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. Methods for designing, optimizing, producing, and using dsRNAs, siRNAs, or shRNAs, are known in the art.

In some aspects, provided herein are therapeutic formulations comprising an anti-cancer therapy provided herein (e.g., an immune checkpoint inhibitor and/or an additional anti-cancer therapy), and a pharmaceutically acceptable carrier, excipient, or stabilizer. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Sustained-release compositions may be prepared. Suitable examples of sustained-release compositions include semi-permeable matrices of solid hydrophobic polymers containing an anti-cancer therapy of the disclosure. Such matrices may be in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

A formulation provided herein may also contain more than one active compound, for example, those with complementary activities that do not adversely affect each other. The type and effective amounts of such medicaments depend, for example, on the amount and type of active compound(s) present in the formulation, and clinical parameters of the subjects.

For general information concerning formulations, see, e.g., Gilman et al. (eds.) The Pharmacological Bases of Therapeutics, 8th Ed., Pergamon Press, 1990; A. Gennaro (ed.), Remington's Pharmaceutical Sciences, 18th Edition, Mack Publishing Co., Pennsylvania, 1990; Avis et al. (eds.) Pharmaceutical Dosage Forms: Parenteral Medications Dekker, New York, 1993; Lieberman et al. (eds.) Pharmaceutical Dosage Forms: Tablets Dekker, New York, 1990; Lieberman et al. (eds.), Pharmaceutical Dosage Forms: Disperse Systems Dekker, New York, 1990; and Walters (ed.) Dermatological and Transdermal Formulations (Drugs and the Pharmaceutical Sciences), Vol 1 19, Marcel Dekker, 2002.

Formulations to be used for in vivo administration are sterile. This is readily accomplished by filtration through sterile filtration membranes or other methods known in the art.

In some embodiments, an immune checkpoint inhibitor is administered as a monotherapy.

In some embodiments, the immune checkpoint inhibitor is a first line immune checkpoint inhibitor. In some embodiments, the immune checkpoint inhibitor is a second line immune checkpoint inhibitor. In some embodiments, an immune checkpoint inhibitor is administered in combination with one or more additional anti-cancer therapies or treatments. In some embodiments, the one or more additional anti-cancer therapies or treatments include one or more anti-cancer therapies described herein. In some embodiments, the methods of the present disclosure comprise administration of any combination of any of the immune checkpoint inhibitors and anti-cancer therapies provided herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an immune checkpoint inhibitor may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an immune checkpoint inhibitor may be administered in conjunction with a radiation therapy.

### IV. EXEMPLARY EMBODIMENTS

The following exemplary embodiments are representative of some aspects of the invention:

Exemplary Embodiment 1: A method of identifying an individual having a squamous cell cancer or a non-small cell lung cancer (NSCLC) who may benefit from a treatment comprising an immune checkpoint inhibitor, the method comprising detecting in a sample from the individual: (a) a somatic loss of heterozygosity (LOH) of one or more human leukocyte antigen class I (HLA-I) genes, or (b) a somatic LOH of one or more HLA-I genes and a high tumor mutational burden (TMB), wherein detection in the sample of the somatic LOH of one or more HLA-I genes, or of the somatic LOH of one or more HLA-I genes and high TMB, identifies the individual as one who may benefit from a treatment comprising an immune checkpoint inhibitor.

Exemplary Embodiment 2: A method of detecting the presence or absence of a squamous cell cancer or a NSCLC in an individual, the method comprising: (a) detecting the presence or absence of a squamous cell cancer or a NSCLC in a sample from the individual; and (b) detecting in a sample from the individual the presence or absence of: (i) a somatic LOH of one or more HLA-I genes, or (ii) a somatic LOH of one or more HLA-I genes and a high TMB.

Exemplary embodiment 3: A method of selecting a therapy for an individual having a squamous cell cancer or a NSCLC, the method comprising detecting in a sample from the individual: (a) a somatic LOH of one or more HLA-I genes, or (b) a somatic LOH of one or more HLA-I genes and a high TMB, wherein detection in the sample of the somatic LOH of one or more HLA-I genes, or of the somatic LOH of one or more HLA-I genes and high TMB, identifies the individual as one who may benefit from a treatment comprising an immune checkpoint inhibitor.

Exemplary embodiment 4: A method of identifying one or more treatment options for an individual having a squamous cell cancer or a NSCLC, the method comprising: (a) detecting in a sample from the individual: (i) a somatic LOH of one or more HLA-I genes, or (ii) a somatic LOH of one or more HLA-I genes and a high TMB; and (b) generating a report comprising one or more treatment options identified for the individual based at least in part on the detection in the sample of the somatic LOH of one or more HLA-I genes, or of the somatic LOH of one or more HLA-I genes and high TMB, wherein the one or more treatment options comprise an immune checkpoint inhibitor.

Exemplary embodiment 5: A method of identifying one or more treatment options for an individual having a squamous cell cancer or a NSCLC, the method comprising: (a) acquiring knowledge of: (i) a somatic LOH of one or more HLA-I genes, or (ii) a somatic LOH of one or more HLA-I genes and a high TMB, in a sample from the individual; and (b) generating a report comprising one or more treatment options identified for the individual based at least in part on said knowledge, wherein the one or more treatment options comprise an immune checkpoint inhibitor.

Exemplary embodiment 6: A method of selecting a treatment for an individual having a squamous cell cancer or a NSCLC, comprising: acquiring knowledge of: (i) a somatic LOH of one or more HLA-I genes, or (ii) a somatic LOH of one or more HLA-I genes and a high TMB, in a sample from the individual; wherein responsive to the acquisition of said knowledge: (i) the individual is classified as a candidate to receive a treatment comprising an immune checkpoint inhibitor; and/or (ii) the individual is identified as likely to respond to a treatment that comprises an immune checkpoint inhibitor.

Exemplary embodiment 7: A method of predicting survival of an individual having a squamous cell cancer or a NSCLC, comprising: acquiring knowledge of: (i) a somatic LOH of one or more HLA-I genes, or (ii) a somatic LOH of one or more HLA-I genes and a high TMB, in a sample from the individual, wherein responsive to the acquisition of said knowledge, the individual is predicted to have longer survival when treated with a treatment comprising an immune checkpoint inhibitor, as compared to survival of an individual whose squamous cell cancer or NSCLC does not comprise the somatic LOH of one or more HLA-I genes, or the somatic LOH of one or more HLA-I genes and high TMB.

Exemplary embodiment 8: A method of predicting survival of an individual having a squamous cell cancer or a NSCLC treated with an immune checkpoint inhibitor, the method comprising: acquiring knowledge of: (i) a somatic LOH of one or more HLA-I genes, or (ii) a somatic LOH of one or more HLA-I genes and a high TMB, in a sample from the individual, wherein responsive to the acquisition of said knowledge, the individual is predicted to have longer survival after a treatment comprising an immune checkpoint inhibitor, as compared to an individual whose cancer does not exhibit the somatic LOH of one or more HLA-I genes, or the somatic LOH of one or more HLA-I genes and high TMB.

Exemplary embodiment 9: A method of treating or delaying progression of a squamous cell cancer or a NSCLC, comprising: (a) acquiring knowledge of: (i) a somatic LOH of one or more HLA-I genes, or (ii) a somatic LOH of one or more HLA-I genes and a high TMB, in a sample from an individual having a squamous cell cancer or a NSCLC; and (b) responsive to said knowledge, administering to the individual an effective amount of a treatment that comprises an immune checkpoint inhibitor.

Exemplary embodiment 10: A method of treating or delaying progression of a squamous cell cancer or a NSCLC, comprising, responsive to acquiring knowledge of: (i) a somatic LOH of one or more HLA-I genes, or (ii) a somatic LOH of one or more HLA-I genes and a high TMB, in a sample from an individual having a squamous cell cancer or a NSCLC, administering to the individual an effective amount of a treatment that comprises an immune checkpoint inhibitor.

Exemplary embodiment 11: A method of monitoring, evaluating, or screening an individual having a squamous cell cancer or a NSCLC, comprising: acquiring knowledge of: (i) a somatic LOH of one or more HLA-I genes, or (ii) a somatic LOH of one or more HLA-I genes and a high TMB, in a sample from the individual, wherein responsive to the acquisition of said knowledge, the individual is predicted to have longer survival when treated with a treatment comprising an immune checkpoint inhibitor, as compared to an individual whose squamous cell cancer or NSCLC does not comprise the somatic LOH of one or more HLA-I genes, or the somatic LOH of one or more HLA-I genes and high TMB.

Exemplary embodiment 12: A method of treating or delaying progression of a squamous cell cancer or a NSCLC, comprising: (a) detecting in a sample from an individual having a squamous cell cancer or a NSCLC: (i) a somatic LOH of one or more HLA-I genes, or (ii) a somatic LOH of one or more HLA-I genes and a high TMB; and (b) administering to the individual an effective amount of a treatment that comprises an immune checkpoint inhibitor.

Exemplary embodiment 13: A method of assessing a squamous cell cancer or a NSCLC in an individual, the method comprising: (a) detecting in a sample from the individual: (i) a somatic LOH of one or more HLA-I genes, or (ii) a somatic LOH of one or more HLA-I genes and a high TMB; and (b) providing an assessment of the somatic LOH of one or more HLA-I genes, or the somatic LOH of one or more HLA-I genes and high TMB, in the squamous cell cancer or NSCLC.

Exemplary embodiment 14: The method of any one of embodiments 5-11, wherein the acquiring knowledge of the somatic LOH of one or more HLA-I genes, or of the somatic LOH of one or more HLA-I genes and high TMB, comprises detecting the somatic LOH of one or more HLA-I genes, or the somatic LOH of one or more HLA-I genes and high TMB, in a sample from the individual.

Exemplary embodiment 15: The method of any one of embodiments 1-4 and 12-14, wherein detecting somatic LOH of one or more HLA-I genes comprises:
providing a plurality of nucleic acids obtained from a sample from the individual, wherein the plurality of nucleic acids comprises nucleic acids encoding an HLA-I gene;
optionally, ligating one or more adaptors onto one or more nucleic acids from the plurality of nucleic acids;
amplifying nucleic acids from the plurality of nucleic acids;
capturing a plurality of nucleic acids corresponding to the HLA-I gene, wherein the plurality of nucleic acids corresponding to the HLA-I gene is captured from the amplified nucleic acids by hybridization with a bait molecule;
sequencing, by a sequencer, the captured nucleic acids to obtain a plurality of sequence reads corresponding to the HLA-I gene;
fitting, by one or more processors, one or more values associated with one or more of the plurality of sequence reads to a model; and
based on the model, detecting the somatic LOH of one or more HLA-I genes and a relative binding propensity for an HLA allele of the HLA-I gene.

Exemplary embodiment 16: The method of embodiment 15, wherein the somatic LOH of one or more HLA-I genes and relative binding propensity for an HLA allele of the HLA-I gene are detected by:
(a) obtaining an observed allele frequency for an HLA allele of an HLA-I gene, wherein the observed allele frequency corresponds to the frequency of nucleic acid(s) encoding at least a portion of the HLA allele as detected among the plurality of sequence reads corresponding to the HLA-I gene;
(b) obtaining a relative binding propensity for the HLA allele to the bait molecule, wherein the relative binding propensity of the HLA allele corresponds to propensity of a nucleic acid encoding at least a portion of the HLA allele to bind the bait molecule in the presence of nucleic acids encoding portions of one or more other HLA alleles;
(c) applying an objective function to measure a difference between the relative binding propensity and the observed allele frequency of the HLA allele;
(d) applying an optimization model to minimize the objective function;
(e) determining an adjusted allele frequency of the HLA allele based on the optimization model and the observed allele frequency; and
(f) determining that LOH of one or more HLA-I genes has occurred when the adjusted allele frequency of the HLA allele is less than a predetermined threshold.

Exemplary embodiment 17: The method of any one of embodiment 1-4 and 12-14, wherein detecting somatic LOH of one or more HLA-I genes comprises determining the specific copy number of an HLA allele of the one or more HLA-I genes in the squamous cell cancer or NSCLC.

Exemplary embodiment 18: The method of embodiment 17, comprising:
(a) aligning a plurality of sequence reads of an HLA allele of one or more HLA-I genes with reference sequence reads of an HLA allele of one or more HLA-I genes, wherein the plurality of sequence reads is derived from a sample of the squamous cell cancer or NSCLC, and wherein the reference sequence reads are based on the individual's HLA type;
(b) determining mismatch positions in homologous HLA alleles of the one or more HLA-I genes, and determining mismatch coverage for each HLA allele;
(c) determining the ratio and allele frequency of each HLA allele based on mismatches and coverage determined in step (b); and
(d) determining the copy number of each HLA allele in the squamous cell cancer or NSCLC based on the ratio and allele frequency determined in step (c).

Exemplary embodiment 19: The method of any one of embodiments 15-18, wherein the plurality of sequence reads is obtained by whole exome sequencing, whole genome sequencing, gene-targeted sequencing, or next-generation sequencing.

Exemplary embodiment 20: The method of any one of embodiments 15-18, wherein the plurality of sequence reads is obtained by next-generation sequencing.

Exemplary embodiment 21: The method of any one of embodiments 1-4 and 12-14, wherein somatic LOH of one or more HLA-I genes is detected by sequencing.

Exemplary embodiment 22: The method of embodiment 21, wherein somatic LOH of one or more HLA-I genes is detected by whole exome sequencing, whole genome sequencing, gene-targeted sequencing, or next-generation sequencing.

Exemplary embodiment 23: The method of any one of embodiments 1-4 and 12-14, wherein somatic LOH of one or more HLA-I genes is detected by next-generation sequencing.

Exemplary embodiment 24: The method of any one of embodiments 1-23, wherein the one or more HLA-I genes comprise one or more of a human HLA-A, HLA-B or HLA-C gene.

Exemplary embodiment 25: The method of any one of embodiments 1-24, wherein a high TMB comprises a TMB of at least about 10 mutations/megabase (mut/Mb).

Exemplary embodiment 26: The method of any one of embodiments 1-25, wherein the high TMB is detected by sequencing, whole exome sequencing, whole genome sequencing, gene-targeted sequencing, or next-generation sequencing.

Exemplary embodiment 27: The method of any one of embodiments 1-26, wherein the squamous cell cancer or NSCLC is PD-L1-positive.

Exemplary embodiment 28: The method of embodiment 27, wherein the squamous cell cancer or NSCLC has a tumor proportion score of at least about 1%.

Exemplary embodiment 29: The method of embodiment 27, wherein at least about 1% of tumor cells in a sample obtained from the squamous cell cancer or NSCLC are PD-L1-positive.

Exemplary embodiment 30: The method of any one of embodiments 27-29, wherein PD-L1 positivity is assessed by immunohistochemistry.

Exemplary embodiment 31: The method of any one of embodiments 27-30, wherein PD-L1 positivity is assessed in a sample comprising squamous cell cancer or NSCLC cells obtained from the individual.

Exemplary embodiment 32: The method of any one of embodiments 1-31, wherein the squamous cell cancer or NSCLC has a tumor mutational burden of at least about 10 mut/Mb.

Exemplary embodiment 33: The method of any one of embodiments 1-32, wherein the squamous cell cancer or NSCLC does not comprise a mutation in an EGFR gene and/or an ALK gene.

Exemplary embodiment 34: The method of any one of embodiments 1-32, wherein the squamous cell cancer or NSCLC is EGFR-wild type and/or ALK-wild type.

Exemplary embodiment 35: The method of any one of embodiments 1-32, wherein the squamous cell cancer or NSCLC does not comprise a pathogenic mutation in an EGFR gene and/or an ALK gene.

Exemplary embodiment 36: The method of any one of embodiments 1-35, wherein the squamous cell cancer or NSCLC is an advanced squamous cell cancer or NSCLC.

Exemplary embodiment 37: The method of any one of embodiments 1-36, wherein the squamous cell cancer or NSCLC is a metastatic squamous cell cancer or NSCLC.

Exemplary embodiment 38: The method of any one of embodiments 1-37, wherein the NSCLC is an adenocarcinoma, a squamous cell cancer, a large cell cancer, an undifferentiated cancer, a carcinoid tumor, a pleomorphic salivary gland cancer, an adenosquamous cancer, sarcomatoid cancer, or an unclassified carcinoma.

Exemplary embodiment 39: The method of embodiment 38, wherein the NSCLC is an adenocarcinoma or a squamous cell cancer.

Exemplary embodiment 40: The method of any one of embodiments 1-37, wherein the squamous cell cancer is a skin, lip, mouth, esophageal, head and neck, urinary tract, thyroid, penis, prostate, bladder, lung, vaginal, or cervical cancer.

Exemplary embodiment 41: The method of embodiment 40, wherein the squamous cell cancer is a non-melanoma skin cancer.

Exemplary embodiment 42: The method of embodiment 40, wherein the squamous cell cancer is a head and neck cancer.

Exemplary embodiment 43: The method of embodiment 40, wherein the squamous cell cancer is an esophageal cancer.

Exemplary embodiment 44: The method of embodiment 40, wherein the squamous cell cancer is a squamous cell lung cancer.

Exemplary embodiment 45: The method of embodiment 44, wherein the squamous cell lung cancer comprises a mutation in a CDKN2A gene, a SOX2 gene, an LRP1B gene, a BRCA1 gene, an FGF12 gene, a TERC gene, a PIK3CA gene, a PRKCI gene, a PTEN gene, an ARID1A gene, a KDM5A gene, a SPTA1 gene, a FAS gene, an FUBP1 gene, or any combination thereof.

Exemplary embodiment 46: The method of embodiment 44 or embodiment 45, wherein the squamous cell lung cancer comprises a tobacco signature.

Exemplary embodiment 47: The method of any one of embodiments 44-46, wherein the squamous cell lung cancer is a non-small cell lung cancer (NSCLC).

Exemplary embodiment 48: The method of any one of embodiments 1-47, wherein the squamous cell cancer or NSCLC was previously treated with an immune checkpoint inhibitor.

Exemplary embodiment 49: The method of any one of embodiments 1-47, wherein the squamous cell cancer or NSCLC was previously treated with an anti-cancer therapy other than an immune checkpoint inhibitor.

Exemplary embodiment 50: The method of any one of embodiments 1-47, wherein the squamous cell cancer or NSCLC was not previously treated with an immune checkpoint inhibitor.

Exemplary embodiment 51: The method of any one of embodiments 1-47, wherein the squamous cell cancer or NSCLC was not previously treated with an anti-cancer therapy other than an immune checkpoint inhibitor.

Exemplary embodiment 52: The method of any one of embodiments 1-47, wherein the squamous cell cancer or NSCLC was not previously treated.

Exemplary embodiment 53: The method of any one of embodiments 1-47, wherein the squamous cell cancer or NSCLC was previously treated with a first line anti-cancer therapy for squamous cell cancer or NSCLC.

Exemplary embodiment 54: The method of embodiment 53, wherein the first line anti-cancer therapy comprises carboplatin, paclitaxel, paclitaxel protein-bound, gemcitabine, docetaxel, ramucirumab, or any combination thereof.

Exemplary embodiment 55: The method of any one of embodiments 1-47, wherein the squamous cell cancer or NSCLC was previously treated with a second line anti-cancer therapy for squamous cell cancer or NSCLC.

Exemplary embodiment 56: The method of any one of embodiments 1-47, wherein the squamous cell cancer or NSCLC was previously treated with a first line immune checkpoint inhibitor for squamous cell cancer or NSCLC.

Exemplary embodiment 57: The method of any one of embodiments 1-47, wherein the squamous cell cancer or NSCLC was previously treated with a second line immune checkpoint inhibitor for squamous cell cancer or NSCLC.

Exemplary embodiment 58: The method of any one of embodiments 1, 3-12, and 14-47, wherein the immune checkpoint inhibitor is a monotherapy.

Exemplary embodiment 59: The method of any one of embodiments 1, 3-12, 14-47, and 58, wherein the immune checkpoint inhibitor is a first line immune checkpoint inhibitor.

Exemplary embodiment 60: The method of any one of embodiments 1, 3-12, 14-47, and 58-59, wherein the immune checkpoint inhibitor is a second line immune checkpoint inhibitor.

Exemplary embodiment 61: The method of any one of embodiments 1, 3-12, 14-47, and 58-60, wherein the immune checkpoint inhibitor is a PD-1- or a PD-L1-targeted agent.

Exemplary embodiment 62: The method of embodiment 61, wherein the immune checkpoint inhibitor is a PD-1 inhibitor.

Exemplary embodiment 63: The method of embodiment 62, wherein the immune checkpoint inhibitor comprises one or more of nivolumab, pembrolizumab, cemiplimab, or dostarlimab.

Exemplary embodiment 64: The method of embodiment 61, wherein the immune checkpoint inhibitor is a PD-L1-inhibitor.

Exemplary embodiment 65: The method of embodiment 64, wherein the immune checkpoint inhibitor comprises one or more of atezolizumab, avelumab, or durvalumab.

Exemplary embodiment 66: The method of any one of embodiments 1, 3-12, 14-47, and 58-60, wherein the immune checkpoint inhibitor is a CTLA-4 inhibitor.

Exemplary embodiment 67: The method of embodiment 66, wherein the CTLA-4 inhibitor comprises ipilimumab.

Exemplary embodiment 68: The method of any one of embodiments 1-67, wherein the treatment or the one or more treatment options further comprise an additional anti-cancer therapy.

Exemplary embodiment 69: The method of embodiment 68, wherein the additional anti-cancer therapy comprises one or more of a small molecule inhibitor, a chemotherapeutic agent, a cancer immunotherapy, an antibody, a cellular therapy, a nucleic acid, a surgery, a radiotherapy, an anti-angiogenic therapy, an anti-DNA repair therapy, an anti-inflammatory therapy, an anti-neoplastic agent, a growth inhibitory agent, a cytotoxic agent, or any combination thereof.

Exemplary embodiment 70: The method of embodiment 69, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy.

Exemplary embodiment 71: The method of embodiment 69, wherein the nucleic acid comprises a double-stranded RNA (dsRNA), a small interfering RNA (siRNA), or a small hairpin RNA (shRNA).

Exemplary embodiment 72: The method of any one of embodiments 1-71, wherein the sample is obtained from the squamous cell cancer or NSCLC.

Exemplary embodiment 73: The method of embodiment 72, wherein the sample comprises cells from the squamous cell cancer or NSCLC and/or nucleic acids from the squamous cell cancer or NSCLC.

Exemplary embodiment 74: The method of embodiment 73, wherein the sample further comprises non-squamous cell cancer or non-NSCLC cells and/or non-squamous cell cancer or non-NSCLC nucleic acids.

Exemplary embodiment 75: The method of embodiment 73, wherein the sample is from a tumor biopsy, tumor specimen, or circulating tumor cell.

Exemplary embodiment 76: The method of embodiment 73, wherein the sample comprises cell-free DNA (cfDNA) or circulating tumor DNA (ctDNA) from the squamous cell cancer or NSCLC.

Exemplary embodiment 77: The method of embodiment 73, wherein the sample comprises fluid, cells, or tissue.

Exemplary embodiment 78: The method of embodiment 77, wherein the sample comprises blood or plasma.

Exemplary embodiment 79: The method of embodiment 73, wherein the sample is a nucleic acid sample.

Exemplary embodiment 80: The method of embodiment 79, wherein the nucleic acid sample comprises mRNA, DNA, circulating tumor DNA, cell-free DNA, or cell-free RNA.

Exemplary embodiment 81: The method of any one of embodiments 1-80, wherein the individual is a human.

Exemplary embodiment 82: An immune checkpoint inhibitor for use in a method of treating or delaying progression of a squamous cell cancer or NSCLC, wherein the method comprises administering the immune checkpoint inhibitor to an individual, wherein a somatic LOH of one or more HLA-I genes, or a somatic LOH of one or more HLA-I genes and high TMB are detected in a sample derived from a squamous cell cancer or NSCLC in the individual.

Exemplary embodiment 83: An immune checkpoint inhibitor for use in the manufacture of a medicament for treating or delaying progression of a squamous cell cancer or NSCLC, wherein the medicament is to be administered to an individual, wherein a somatic LOH of one or more HLA-I genes, or a somatic LOH of one or more HLA-I genes and high TMB are detected in a sample derived from a squamous cell cancer or NSCLC in the individual.

Exemplary embodiment 84: A system, comprising: a memory configured to store one or more program instructions; and one or more processors configured to execute the one or more program instructions, wherein the one or more program instructions when executed by the one or more processors are configured to:
obtain a plurality of sequence reads of one or more nucleic acids, wherein the one or more nucleic acids are derived from a sample of a squamous cell cancer or NSCLC obtained from an individual;
analyze the plurality of sequence reads for the presence of a somatic LOH of one or more HLA-I genes, or a somatic LOH of one or more HLA-I genes and a high TMB;
detect, based on the analyzing, the presence of a somatic LOH of one or more HLA-I genes, or a somatic LOH of one or more HLA-I genes and high TMB, in the sample; and
generate, based at least in part on the detecting, a genomic profile for the sample.

Exemplary embodiment 85: The system of embodiment 84, wherein the analyzing comprises:
determining an observed allele frequency for an HLA allele of an HLA-I gene, wherein the observed allele frequency corresponds to the frequency of nucleic acid(s) encoding at least a portion of the HLA allele as detected among a plurality of sequence reads corresponding to an HLA-I gene, wherein the plurality of sequence reads was obtained by sequencing nucleic acids encoding the HLA-I gene or a portion thereof as captured by hybridization with a bait molecule;
determining a relative binding propensity for the HLA allele to the bait molecule, wherein the relative binding propensity of the HLA allele corresponds to propensity of a nucleic acid encoding at least a portion of the HLA allele to bind the bait molecule in the presence of nucleic acids encoding portions of one or more other HLA alleles;
executing an objective function to measure a difference between the relative binding propensity and the observed allele frequency of the HLA allele;
executing an optimization model to minimize the objective function;
determining an adjusted allele frequency of the HLA allele based on the optimization model and the observed allele frequency; and
determining the presence of a somatic LOH of one or more HLA-I genes when the adjusted allele frequency of the HLA allele is less than a predetermined threshold.

Exemplary embodiment 86: A non-transitory computer readable storage medium comprising one or more programs executable by one or more computer processors for performing a method, comprising:
obtaining, using the one or more processors, a plurality of sequence reads of one or more nucleic acids, wherein the one or more nucleic acids are derived from a sample of a squamous cell cancer or NSCLC obtained from an individual;
analyzing, using the one or more processors, the plurality of sequence reads for the presence of a somatic LOH of one or more HLA-I genes, or a somatic LOH of one or more HLA-I genes and a high TMB;
detecting, using the one or more processors and based on the analyzing, the presence of a somatic LOH of one or more HLA-I genes, or of a somatic LOH of one or more HLA-I genes and high TMB, in the sample; and
generating, based at least in part on the detecting, a genomic profile for the sample.

Exemplary embodiment 87: The non-transitory computer readable storage medium of embodiment 86, wherein the analyzing comprises:
receiving, using the one or more processors, an observed allele frequency for an HLA allele of an HLA-I gene, wherein the observed allele frequency corresponds to the frequency of nucleic acid(s) encoding at least a portion of the HLA allele as detected among a plurality of sequence reads corresponding to an HLA-I gene, wherein the plurality of sequence reads was obtained by sequencing nucleic acids encoding the HLA-I gene or a portion thereof as captured by hybridization with a bait molecule;
receiving, using the one or more processors, a relative binding propensity for the HLA allele to the bait molecule, wherein the relative binding propensity of the HLA allele corresponds to propensity of a nucleic acid encoding at least a portion of the HLA allele to bind the bait molecule in the presence of nucleic acids encoding portions of one or more other HLA alleles;
executing, using the one or more processors, an objective function to measure a difference between the relative binding propensity and the observed allele frequency of the HLA allele;
executing, using the one or more processors, an optimization model to minimize the objective function;
determining, using the one or more processors, an adjusted allele frequency of the HLA allele based on the optimization model and the observed allele frequency; and
determining, using the one or more processors, that a somatic LOH of one or more HLA-I genes has occurred when the adjusted allele frequency of the HLA allele is less than a predetermined threshold.

Exemplary embodiment 88: The system of embodiment 84 or embodiment 85, or the non-transitory computer readable storage medium of embodiment 86 or embodiment 87, wherein the one or more HLA-I genes comprise one or more of a human HLA-A, HLA-B, or HLA-C gene.

Exemplary embodiment 89: The system of any one of embodiments 84-85 and 88, or the non-transitory computer readable storage medium of any one of embodiments 86-87 and 88, wherein the plurality of sequence reads is obtained by sequencing nucleic acids obtained from a sample comprising squamous cell cancer or NSCLC cells and/or squamous cell cancer or NSCLC nucleic acids.

Exemplary embodiment 90: The system or non-transitory computer readable storage medium of embodiment 89, wherein the plurality of sequence reads is obtained by whole exome sequencing, whole genome sequencing, gene-targeted sequencing, or next-generation sequencing.

Exemplary embodiment 91: The system or non-transitory computer readable storage medium of embodiment 89 or embodiment 90, wherein the sample further comprises non-squamous cell cancer or non-NSCLC cells and/or non-squamous cell cancer or non-NSCLC nucleic acids.

Exemplary embodiment 92: The system of any one of embodiments 84-85 and 88-91, or the non-transitory computer readable storage medium of any one of embodiments 86-87 and 88-91, wherein the sample is from a tumor biopsy, tumor specimen, or a circulating tumor cell.

Exemplary embodiment 93: The system of any one of embodiments 84-85 and 88-91, or the non-transitory computer readable storage medium of any one of embodiments 86-87 and 88-91, wherein the sample comprises cell-free DNA (cfDNA) or circulating tumor DNA (ctDNA) from the squamous cell cancer or NSCLC.

Exemplary embodiment 94: The system of any one of embodiments 84-85 and 88-91, or the non-transitory computer readable storage medium of any one of embodiments 86-87 and 88-91, wherein the sample comprises fluid, cells, or tissue.

Exemplary embodiment 95: The system or non-transitory computer readable storage medium of embodiment 94, wherein the sample comprises blood or plasma.

Exemplary embodiment 96: The system of any one of embodiments 84-85 and 88-91, or the non-transitory computer readable storage medium of any one of embodiments 86-87 and 88-91, wherein the sample is a nucleic acid sample.

Exemplary embodiment 97: The system or non-transitory computer readable storage medium of embodiment 96, wherein the nucleic acid sample comprises mRNA, DNA, circulating tumor DNA, cell-free DNA, or cell-free RNA.

Exemplary embodiment 98: The system of any one of embodiments 84-85 and 88-97, or the non-transitory computer readable storage medium of any one of embodiments 86-87 and 88-97, wherein a high TMB comprises a TMB of at least about 10 mut/Mb.

Exemplary embodiment 99: The system of any one of embodiments 84-85 and 88-98, or the non-transitory computer readable storage medium of any one of embodiments 86-87 and 88-98, wherein the individual is administered a treatment based at least in part on the genomic profile.

The method steps of the invention(s) described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The disclosures of all publications, patents, and patent applications referred to herein are each hereby incorporated by reference in their entireties. To the extent that any reference incorporated by reference conflicts with the instant disclosure, the instant disclosure shall control.

### Examples

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Somatic HLA-I Loss of Heterozygosity as a Biomarker for Improved Survival in Immune Checkpoint Inhibitor-Treated Patients with Squamous Cell Lung Carcinoma

This Example describes the characterization of loss of heterozygosity (LOH) of a human leukocyte antigen class I (HLA-I) gene and tumor mutational burden (TMB) in squamous cell lung carcinoma (lung SCC) as biomarkers predictive of responses to immune checkpoint inhibitor therapy.

### Materials and Methods

The results described in this Example were based on analysis of EGFR- and ALK-wild type squamous cell lung carcinoma cases, including squamous non-small cell lung cancers (squamous NSCLC), in a clinico-genomics database.

HLA-I LOH was assessed as described in Montesion, M., et al., Cancer Discovery (2021) 11(2):282-92, using a somatic-germline-zygosity (SGZ) algorithm. The SGZ is a computational method for zygosity prediction from next-generation sequencing results of mixed tumor-normal samples (20%-95% tumor), from pipeline v3.1.3. See, Sun et al., PLoS Comput Biol (2018) 14:e1005965.

TMB was defined as the number of non-driver somatic coding mutations/megabase (mut/Mb) of genome sequenced. Mutational signatures were determined in samples with ≥20 non-driver somatic mutations, including silent and noncoding alterations. TMB high status was defined as TMB ≥10 mut/Mb. See, Montesion, M., et al., Cancer Discovery (2021) 11(2):282-92.

PD-L1 expression was assessed by immunohistochemistry (IHC) using commercially available antibody clones 22C3 (Dako/Agilent) or SP142 (Ventana). PD-L1 expression for each sample was summarized as negative (<1% of tumor cells) or positive (≥1% of tumor cells). See, Montesion, M., et al., Cancer Discovery (2021) 11(2):282-92.

Survival was assessed using Kaplan-Meier analyses, with the log-rank test used to compare groups. Statistics on patient demographics were conducted by a two-sided Fisher exact test. Analyses were performed on the R software version 3.6.0. See, Montesion, M., et al., Cancer Discovery (2021)11(2):282-92.

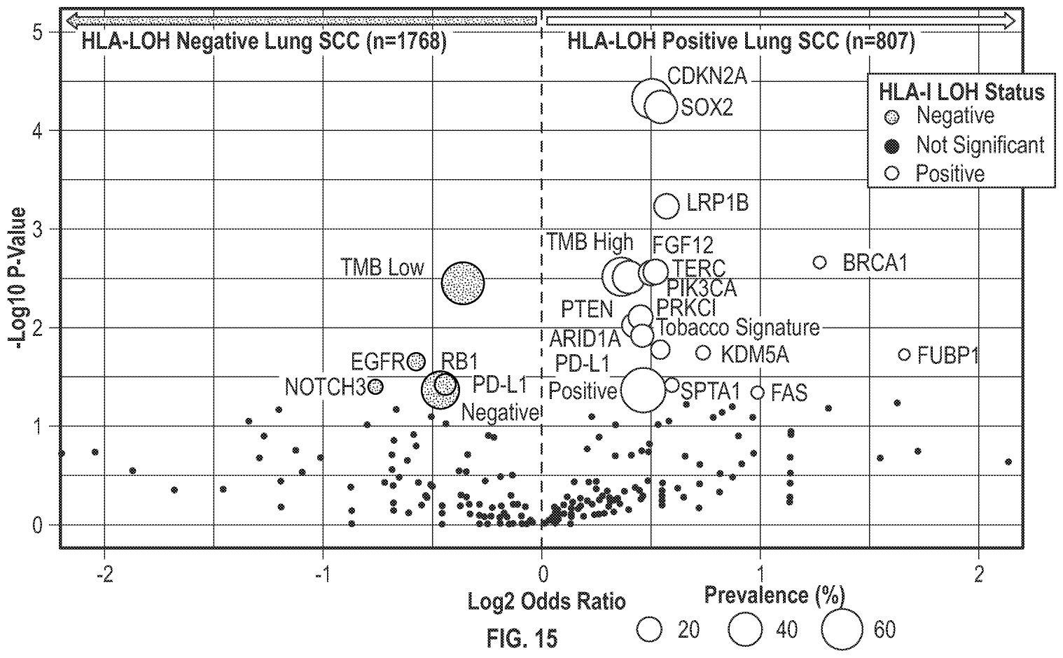

For analysis of genomic alterations, e.g., as shown in FIG. 15, hybridization-capture-based next-generation sequencing was performed for all coding exons of 315 genes plus 28 introns frequently rearranged in cancer. See, Frampton et al., Nat Biotechnol (2013) 31:1023-31. Libraries were sequenced to a median unique coverage depth of >500×. Genomic alterations analyzed included short variant alterations (base substitutions, insertions, and deletions), copy-number alterations (amplifications and homozygous deletions), as well as gene rearrangements.

### Results

To investigate whether HLA-I LOH is a predictive biomarker for ICI response in squamous cell lung carcinoma (lung SCC), a clinico-genomics database of EGFR- and ALK-wild-type lung SCC cases (including squamous NSCLC) was analyzed and stratified with respect to presence of HLA-I LOH. The presence of an HLA-I LOH was defined as at least one HLA-I gene (HLA-A, HLA-B, or HLA-C) being under LOH in the tumor sample. Prior studies have shown that HLA-I LOH has a pan-cancer prevalence of 17%, and prevalence in squamous cell lung cancer of 31% (Montesion, M., et al., Cancer Discovery (2021) 11(2):282-92).

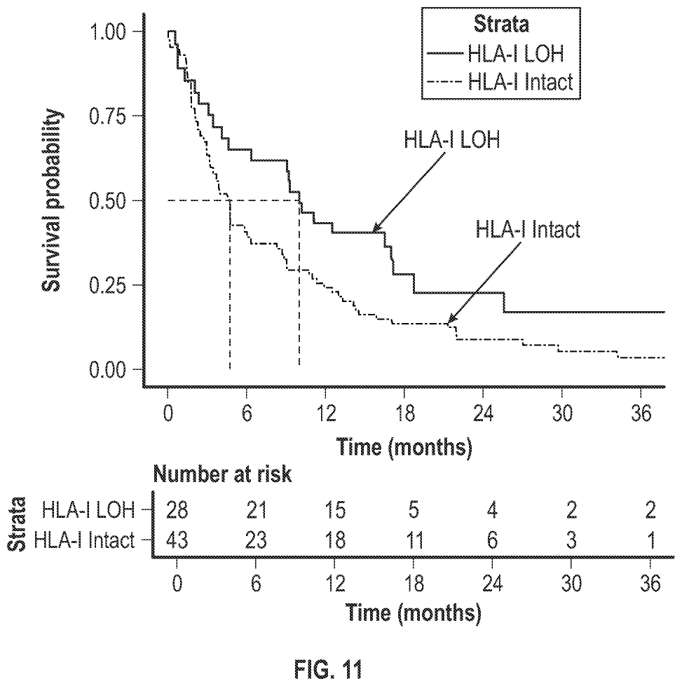

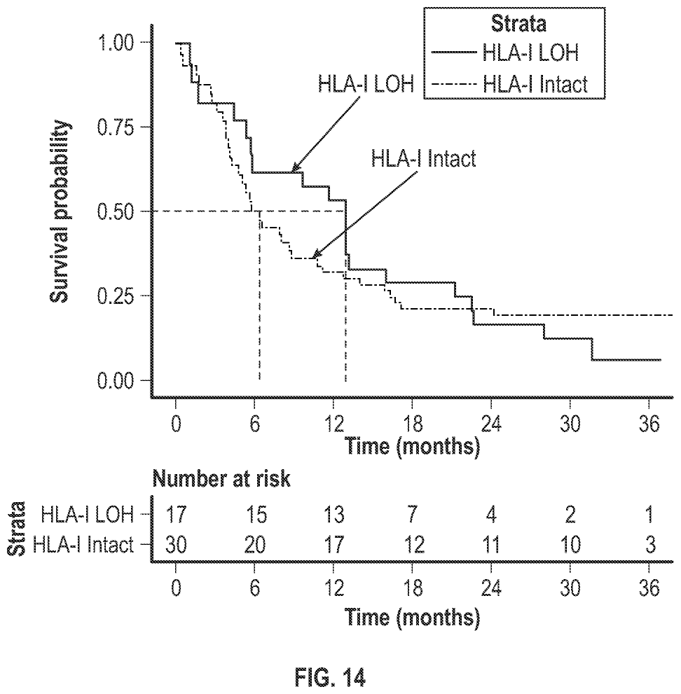

Somatic HLA-I LOH was found to be an independent and significant positive predictor of median overall survival (mOS) from start of second-line ICI monotherapy (FIG. 11). mOS (months) for HLA-I LOH (n=33) was 10.0 months (95% confidence interval (CI): 4.6-18.8); and 4.8 months (95% CI: 3.2-8.7) for HLA-I intact (n=66). Hazard ratio (HR) for HLA-I intact was 1.8 (95% CI: 1.1-2.9); p=0.02). A similar trend was observed in patients treated with first-line ICI monotherapy (FIG. 14; mOS (months) HLA-I LOH (n=24): 13.0 (95% CI: 5.7-22.6); HLA-I intact (n=46): 6.4 (95% CI: 4.2-12.8); HR for HLA-I intact: 1.0 (95% CI: 0.6-1.7); Log-rank P value: 0.9).

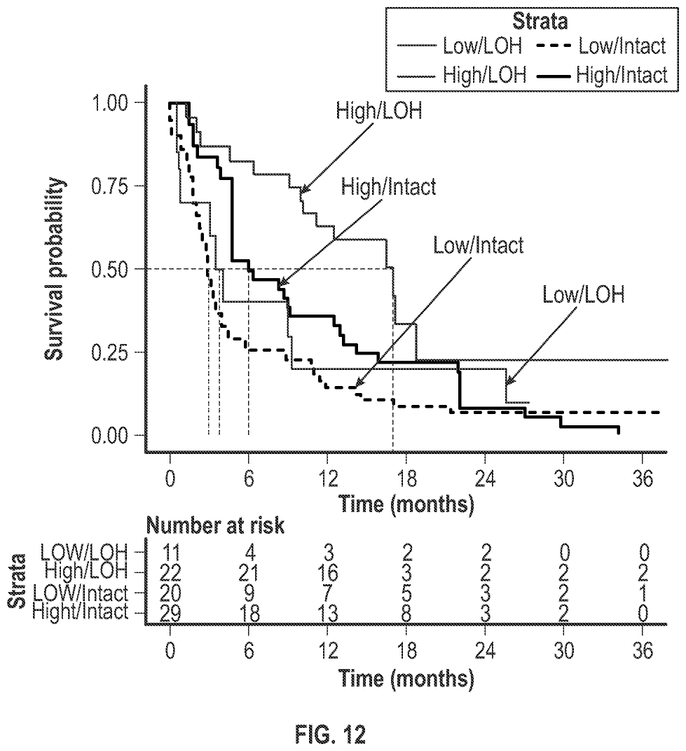

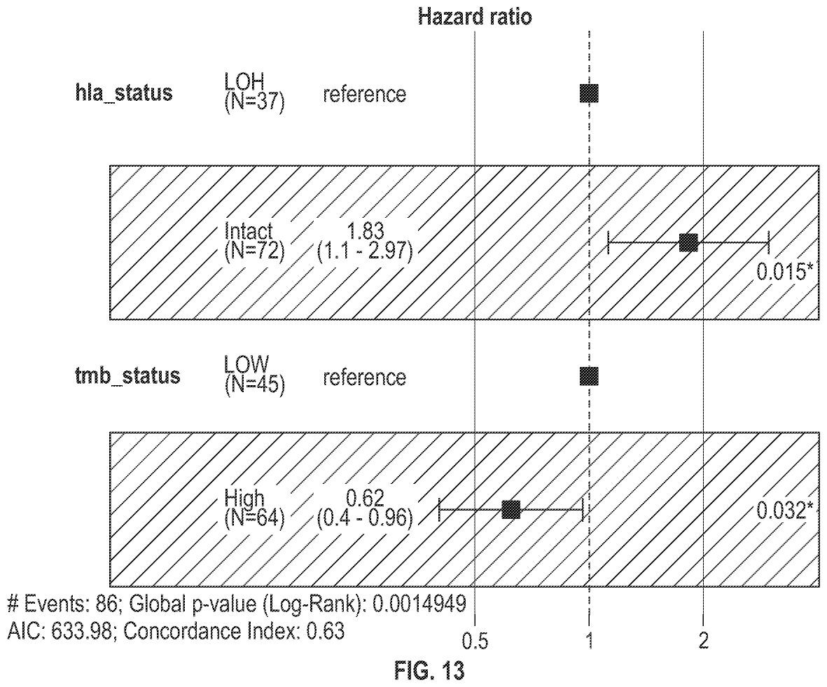

When second-line ICI-treated patients were further stratified by TMB status, a significantly longer median overall survival was observed in patients who were both TMB high and positive for HLA-I LOH as compared to those who were HLA-I intact or who were TMB low and positive for HLA-I LOH (FIG. 12; mOS (months) HLA-I LOH/TMB high (n=25): 17.0 (95% CI: 11.1-NA); HLA-I intact/TMB high (n=39): 6.0 (95% CI: 4.8-13.3); HLA-I LOH/TMB low (n=12): 3.4 (95% CI: 0.8-NA); HLA-I intact/TMB low (n=33): 3.0 (95% CI: 2.1-5.8)). As shown in FIG. 13, the hazard ratio for HLA-I intact was 1.83 (95% CI: 1.1-2.97; p=0.015), while the hazard ratio for TMB high was 0.62 (95% CI: 0.4-0.96; p=0.032).

As shown in FIG. 15, another analysis of lung SCC cases revealed a higher prevalence of tobacco signature, high TMB status, PD-L1 positivity, as well as genomic alterations in PTEN, FGF12, TERC, PIK3CA, BRCA1, FUBP1, FAS, ARID1A, KDMSA, CDKN2A, SOX2, PRKCI, SPTAI, and LRP1B in lung SCC cases with HLA-I LOH. In contrast, HLA-I intact lung SCC exhibited higher prevalence of TMB low status, PD-L1 negative status, as well as genomic alterations in EGFR, NOTCH3 and RB1.

### CONCLUSIONS

The results described in this Example show that squamous cell lung cancer patients that were positive for HLA-I LOH, or positive for HLA-I LOH and high TMB showed improved responses to ICI treatment, as compared to patients without HLA-I LOH, or without HLA-I LOH and high TMB. Thus, HLA-I LOH and TMB status can be used as biomarkers for identifying treatment options and/or predicting responsiveness to ICI treatment for lung SCC patients, including squamous NSCLC patients.

1-8. (canceled)

9. A method of treating or delaying progression of a squamous cell cancer or a NSCLC, comprising:
(a) acquiring knowledge of:
(i) a somatic LOH of one or more HLA-I genes, or
(ii) a somatic LOH of one or more HLA-I genes and a high TMB,
in a sample from an individual having a squamous cell cancer or a NSCLC; and
(b) responsive to said knowledge, administering to the individual an effective amount of a treatment that comprises an immune checkpoint inhibitor.

10. A method of treating or delaying progression of a squamous cell cancer or a NSCLC, comprising,
responsive to acquiring knowledge of:
(i) a somatic LOH of one or more HLA-I genes, or
(ii) a somatic LOH of one or more HLA-I genes and a high TMB,
in a sample from an individual having a squamous cell cancer or a NSCLC,
administering to the individual an effective amount of a treatment that comprises an immune checkpoint inhibitor.

11. (canceled)

12. A method of treating or delaying progression of a squamous cell cancer or a NSCLC, comprising:
(a) detecting in a sample from an individual having a squamous cell cancer or a NSCLC:
(i) a somatic LOH of one or more HLA-I genes, or
(ii) a somatic LOH of one or more HLA-I genes and a high TMB; and
(b) administering to the individual an effective amount of a treatment that comprises an immune checkpoint inhibitor.

13-14. (canceled)

15. The method of claim 12, wherein detecting somatic LOH of one or more HLA-I genes comprises:
providing a plurality of nucleic acids obtained from a sample from the individual, wherein the plurality of nucleic acids comprises nucleic acids encoding an HLA-I gene;
optionally, ligating one or more adaptors onto one or more nucleic acids from the plurality of nucleic acids;
amplifying nucleic acids from the plurality of nucleic acids;
capturing a plurality of nucleic acids corresponding to the HLA-I gene, wherein the plurality of nucleic acids corresponding to the HLA-I gene is captured from the amplified nucleic acids by hybridization with a bait molecule;
sequencing, by a sequencer, the captured nucleic acids to obtain a plurality of sequence reads corresponding to the HLA-I gene;
fitting, by one or more processors, one or more values associated with one or more of the plurality of sequence reads to a model; and
based on the model, detecting the somatic LOH of one or more HLA-I genes and a relative binding propensity for an HLA allele of the HLA-I gene.

16. (canceled)

17. The method of claim 12, wherein detecting somatic LOH of one or more HLA-I genes comprises determining the specific copy number of an HLA allele of the one or more HLA-I genes in the squamous cell cancer or NSCLC.

18-20. (canceled)

21. The method of claim 12, wherein somatic LOH of one or more HLA-I genes is detected by whole exome sequencing, whole genome sequencing, gene-targeted sequencing, or next-generation sequencing.

22-24. (canceled)

25. The method of claim 12, wherein a high TMB comprises a TMB of at least about 10 mutations/megabase (mut/Mb).

26. (canceled)

27. The method of claim 12, wherein the squamous cell cancer or NSCLC is PD-L1-positive.

28-31. (canceled)

32. The method of claim 12, wherein the squamous cell cancer or NSCLC has a tumor mutational burden of at least about 10 mut/Mb.

33. The method of claim 12, wherein:
(a) the squamous cell cancer or NSCLC does not comprise a mutation in an EGFR gene and/or an ALK gene;
(b) the squamous cell cancer or NSCLC is EGFR-wild type and/or ALK-wild type; or
(c) the squamous cell cancer or NSCLC does not comprise a pathogenic mutation in an EGFR gene and/or an ALK gene.

34-35. (canceled)

36. The method of claim 12, wherein the squamous cell cancer or NSCLC is an advanced and/or metastatic squamous cell cancer or NSCLC.

37-39. (canceled)

40. The method of claim 12, wherein the squamous cell cancer is a skin, lip, mouth, esophageal, head and neck, urinary tract, thyroid, penis, prostate, bladder, lung, vaginal, or cervical cancer.

41-43. (canceled)

44. The method of claim 40, wherein the squamous cell cancer is a squamous cell lung cancer.

45. The method of claim 44, wherein:
(a) the squamous cell lung cancer comprises a mutation in a CDKN2A gene, a SOX2 gene, an LRP1B gene, a BRCA1 gene, an FGF12 gene, a TERC gene, a PIK3CA gene, a PRKCI gene, a PTEN gene, an ARID1A gene, a KDM5A gene, a SPTA1 gene, a FAS gene, an FUBP1 gene, or any combination thereof;
(b) the squamous cell lung cancer comprises a tobacco signature; and/or
(c) the squamous cell lung cancer is a non-small cell lung cancer (NSCLC).

46-47. (canceled)

48. The method of claim 12, wherein:
(a) the squamous cell cancer or NSCLC was previously treated with an immune checkpoint inhibitor;
(b) the squamous cell cancer or NSCLC was previously treated with an anti-cancer therapy other than an immune checkpoint inhibitor;
(c) the squamous cell cancer or NSCLC was not previously treated with an immune checkpoint inhibitor;
(d) the squamous cell cancer or NSCLC was not previously treated with an anti-cancer therapy other than an immune checkpoint inhibitor;
(e) the squamous cell cancer or NSCLC was not previously treated;
(f) the squamous cell cancer or NSCLC was previously treated with a first line anti-cancer therapy for squamous cell cancer or NSCLC;
(g) the squamous cell cancer or NSCLC was previously treated with a second line anti-cancer therapy for squamous cell cancer or NSCLC;
(h) the squamous cell cancer or NSCLC was previously treated with a first line immune checkpoint inhibitor for squamous cell cancer or NSCLC: or
(i) the squamous cell cancer or NSCLC was previously treated with a second line immune checkpoint inhibitor for squamous cell cancer or NSCLC.

49-57. (canceled)

58. The method of claim 12, wherein the immune checkpoint inhibitor is:
(a) a monotherapy;
(b) a first line immune checkpoint inhibitor;
(c) a second line immune checkpoint inhibitor; and/or
(d) a PD-1- or a PD-L1-targeted agent or a CTLA-4 inhibitor.

59-71. (canceled)

72. The method of claim 12, wherein the sample is obtained from the squamous cell cancer or NSCLC.

73. The method of claim 72, wherein the sample comprises cells from the squamous cell cancer or NSCLC and/or nucleic acids from the squamous cell cancer or NSCLC.

74. The method of claim 73, wherein:
(a) the sample further comprises non-squamous cell cancer or non-NSCLC cells and/or non-squamous cell cancer or non-NSCLC nucleic acids;
(b) the sample is from a tumor biopsy, tumor specimen, or circulating tumor cell;
(c) the sample comprises cell-free DNA (cfDNA) or circulating tumor DNA (ctDNA) from the squamous cell cancer or NSCLC;
(d) the sample comprises fluid, cells, or tissue;
(e) the sample comprises blood or plasma; or
(f) the sample is a nucleic acid sample comprising mRNA, DNA, circulating tumor DNA, cell-free DNA, or cell-free RNA.

75-80. (canceled)

81. The method of claim 12, wherein the individual is a human.

82-99. (canceled)
